(19) United States
(12) Patent Application Publication
(10) Pub. No.: US 2011/0086395 A1
(43) Pub. Date: Apr. 14, 2011

(54) NOVEL MICROORGANISM AND ITS USE IN LIGNOCELLULOSE DETOXIFICATION

(76) Inventors: Frank Wouter KOOPMAN, Delft (NL); Harald Johan Ruijssenaars, Delft (NL); Nick Johannes Petrus Wierckx, Delft (NL); Johannes Hendrik De Winde, Delft (NL)

(21) Appl. No.: 12/874,913

(22) Filed: Sep. 2, 2010

(30) Foreign Application Priority Data
Sep. 2, 2009 (EP) 09169260.8
Oct. 8, 2009 (EP) 09172567.1

(51) Int. Cl. C12P 7/06; C12N 1/20; C12N 9/06; C07H 21/04; C07K 14/195; C12N 9/88; C12N 9/04; C12N 9/02; C12N 9/00; C12N 9/16; C12N 15/63; C12N 5/10; C12N 1/21; C12N 1/19; C12N 1/15; C12P 19/32

(52) U.S. Cl. 435/92; 435/252.1; 435/191; 536/23.2; 530/350; 435/232; 435/190; 435/189; 435/183; 435/196; 435/320.1; 435/325; 435/419; 435/252.33; 435/252.3; 435/252.31; 435/252.32; 435/252.35; 435/252.34; 435/252.2; 435/254.22; 435/254.2; 435/254.23; 435/254.21; 435/254.3; 435/254.11; 435/254.5; 435/254.6; 435/161

(57) ABSTRACT

An isolated microorganism is provided which is Cupriavidus basilensis strain HMF14 Deposit number DSM 22875, and its use in a process for the in-situ detoxification of lignocelluloses hydrolysate.

### FIELD OF THE INVENTION

The present invention relates to the isolation of a novel Cupriavidus species, and to novel polynucleotide sequences encoding novel polypeptides derived from this organism. The enzymes described herein are useful in the detoxification of lignocellulose hydrolysates in recombinant organisms.

### BACKGROUND OF THE INVENTION

The use of lignocellulose-containing material for the production of second-generation biofuels and biochemicals is well known in the art. Commonly, the process comprises the steps of pretreatment, hydrolysis, and fermentation.

A significant drawback of the pretreatment process step, in particular if involving acidic conditions, is the release of degradation products from the lignocellulose-containing material that may evolve into fermentation inhibitors during the hydrolysis process step. Specifically furanic compounds, such as furfural, furfuryl alcohol, 5-(hydroxymethyl)-furfural (HMF) and/or furoic acid pose problems that are difficult to mitigate, since they inhibit the growth of e.g. ethanologenic microorganisms employed in the fermentation step, thereby inhibiting a suitable performance of these organisms and reducing yields.

Approaches to remove the degradation products at least in part by chemical, physical or biological methods have been discussed in detail in Bioresource Technology 93 (2004), 1-10, and Microbiol. Biotechnol, DOI 10.1007/s00253-009-1875-1, published 31 Jan. 2009.

Yu et al, in their article titled “Microbial utilizaiton and biopolyester synthesis of bagasse hydrolysates”, Bioresource Technology, vol 99 (2008), pages 8042-8048, investigated the utilization of hydrolysates by an aerobic bacterium, Ralstonia eutropha, to determine if organic inhibitors can be removed for potential recycling and reuse of the process water. In the article it is noted that R. Eutropha has a high metabolic activity on furfural. The R. Eutropha, however, also showed high metabolic activity on glucose and fructose.

WO2009/030713 discloses the washing of pre-treated lignocellulose-containing material, further referred to as lignocellulose hydrolysate. However, the inhibitors have a comparatively low solubility in water, while recycling of the wash water is only possible to a very limited extent due to build-up of the inhibitors, thus requiring large amounts of water. This makes the disclosed process cumbersome and difficult to apply on a commercial scale.

WO2009/017441 discloses a process for biological detoxification of lignocellulosic hydrolysate with genetically modified yeasts, while U.S. Pat. No. 7,067,303 discloses the use of a fungus for the same purpose.

While such microorganisms can metabolize the fermentation inhibitors or convert them into less toxic compounds, their preferred carbon source are fermentable sugars, thereby reducing the fermentable sugar content of the lignocellulosic hydrolysate, and thus reducing the overall yield of the desired fermentation products.

Consequently, there is a need for providing a process for detoxifying pre-treated lignocellulose-containing material to obtain substrates suitable for hydrolysis, fermentation or thermochemical conversion, while preserving the fermentable sugar content for subsequent or simultaneous process steps.

### SUMMARY OF THE INVENTION

In one embodiment, the present invention relates to an isolated microorganism of the family Burkholderiaceae, genus Cupriavidus, and the species and strain designation basilensis HMF 14. This strain has been deposited on 19 Aug. 2009 at the Deutsche Sammlung von Mikroorganismen and Zellkulturen GmbH (German Collection of Microorganisms and Cell Cultures), Inhoffenstraβe 7B, 38124 Braunschweig, GERMANY as strain HMF 14, Deposit number DSM 22875.

In another embodiment, the invention relates to a bacterial culture comprising the Cupriavidus microorganism of Cupriavidus basilensis HMF 14 strain Deposit number DSM 22875. This strain preferably grows, when provided with HMF, Furfurylalcohol, Furfural and/or Furoic acid as a carbon source. In another embodiment, the invention further relates to an isolated microorganism or culture according to the invention, which expresses the following enzymes: a Furoyl-CoA dehydrogenase (composed of three polypeptide subunits), a Furoyl-CoA synthetase, a 2-oxoglutaroyl-CoA hydrolase, a 2,5-furan-dicarboxylic acid decarboxylase 1, a 2,5-furan-dicarboxylic acid decarboxylase 2 and a HMF/furfural oxidoreductase. These enzymes form a novel pathway to selectively metabolize furanic compounds.

In another embodiment, the present invention provides polynucleotides encoding polypeptides having the following activities: a 2-furoyl-CoA:acceptor 5-oxidoreductase (hydroxylating), EC 1.3.99.8 (further referred to herein as Furoyl-CoA dehydrogenase); a Furoyl-CoA synthetase, EC 6.2.1.31; a 2-oxoglutaroyl-CoA hydrolase (thioester hydrolase), EC 3.1.2; a first 2,5-furan-dicarboxylic acid decarboxylase (Carboxy-lyase), EC 4.1.1; a second 2,5-furan-dicarboxylic acid decarboxylase (Carboxy-lyase), EC 4.1.1; and a HMF/furfural oxido-reductase EC 1.1.3 and 1.2.3.

In one embodiment, the polynucleotide of the invention typically encodes a polypeptide having Furoyl-CoA dehydrogenase activity (large subunit hmfA). In another embodiment, the polynucleotide of the invention typically encodes a polypeptide having Furoyl-CoA dehydrogenase activity (FAD binding subunit, hmfB). In another embodiment, the polynucleotide of the invention typically encodes a Furoyl-CoA dehydrogenase (2Fe-2S iron sulfur subunit, hmfC). Yet in another embodiment, the polynucleotide of the invention typically encodes a polypeptide having Furoyl-CoA synthetase activity. In another embodiment, the polynucleotide of the invention typically encodes a polypeptide having 2-oxoglutaroyl-CoA hydrolase activity. In another embodiment, the polynucleotide of the invention typically encodes a polypeptide having 2,5-furan-dicarboxylic acid decarboxylase activity, while a further polynucleotide of the invention typically encodes a polypeptide having a second 2,5-furan-dicarboxylic acid decarboxylase activity.

In another embodiment, the invention provides a polypeptide having aldehyde dehydrogenase activity which comprises the amino acid sequence set out in SEQ ID NO: 15 or an amino acid sequence encoded by the nucleotide sequence of SEQ ID NO: 16 or a variant polypeptide thereof, wherein the variant has at least 64% sequence identity or more with the sequence set out in SEQ ID NO: 16.

In another embodiment, the invention further provides a polynucleotide which comprises:
(a) the nucleotide sequence set out in SEQ ID NO: 16;
(b) a nucleotide sequence which hybridizes selectively with a polynucleotide being the reverse complement of SEQ ID NO: 16;
(c) a nucleotide sequence having at least 64% sequence identity or more with the nucleotide sequence of SEQ ID NO: 16;
(d) a fragment of a nucleotide sequence as defined in (a), (b) or (c) which is at least about 100 nucleotides in length;
(e) a sequence which is degenerate as a result of the genetic code to a sequence as defined in any one of (a), (b), (c) or (d);
(f) a nucleotide sequence which is the reverse complement of a nucleotide sequence as defined in (a), (b), (c), (d) or (e).

In another embodiment, the invention further provides a polypeptide having LysR family transcriptional regulator activity which comprises the amino acid sequence set out in SEQ ID NO: 17 or an amino acid sequence encoded by the nucleotide sequence of SEQ ID NO: 18 or a variant polypeptide thereof, wherein the variant has at least 47% sequence identity or more with the sequence set out in SEQ ID NO: 17.

In another embodiment, the invention further provides a polynucleotide which comprises:
(a) the nucleotide sequence set out in SEQ ID NO: 18;
(b) a nucleotide sequence which hybridizes selectively with a polynucleotide being the reverse complement of SEQ ID NO: 18;
(c) a nucleotide sequence having at least 72% sequence identity or more with the nucleotide sequence of SEQ ID NO: 18;
(d) a fragment of a nucleotide sequence as defined in (a), (b) or (c) which is at least about 100 nucleotides in length;
(e) a sequence which is degenerate as a result of the genetic code to a sequence as defined in any one of (a), (b), (c) or (d);
(f) a nucleotide sequence which is the reverse complement of a nucleotide sequence as defined in (a), (b), (c), (d) or (e).

In another embodiment, the invention further provides a polypeptide having 2,5-furan-dicarboxylic acid decarboxylase 1 activity which comprises the amino acid sequence set out in SEQ ID NO: 19 or an amino acid sequence encoded by the nucleotide sequence of SEQ ID NO: 20 or a variant polypeptide thereof, wherein the variant has at least 54% sequence identity or more with the sequence set out in SEQ ID NO: 19.

In another embodiment, the invention further provides a polynucleotide which comprises:
(a) the nucleotide sequence set out in SEQ ID NO: 20;
(b) a nucleotide sequence which hybridizes selectively with a polynucleotide being the reverse complement of SEQ ID NO: 20;
(c) a nucleotide sequence having at least 66% sequence identity or more with the nucleotide sequence of SEQ ID NO: 20;
(d) a fragment of a nucleotide sequence as defined in (a), (b) or (c) which is at least about 100 nucleotides in length;
(e) a sequence which is degenerate as a result of the genetic code to a sequence as defined in any one of (a), (b), (c) or (d);
(f) a nucleotide sequence which is the reverse complement of a nucleotide sequence as defined in (a), (b), (c), (d) or (e).

In another embodiment, the invention further provides a polypeptide having 2,5-furan-dicarboxylic acid decarboxylase 2 activity which comprises the amino acid sequence set out in SEQ ID NO: 21 or an amino acid sequence encoded by the nucleotide sequence of SEQ ID NO: 22 or a variant polypeptide thereof, wherein the variant has at least 52% sequence identity or more with the sequence set out in SEQ ID NO: 21.

In another embodiment, the invention further provides a polynucleotide which comprises:
(a) the nucleotide sequence set out in SEQ ID NO: 22;
(b) a nucleotide sequence which hybridizes selectively with a polynucleotide being the reverse complement of SEQ ID NO: 22;
(c) a nucleotide sequence having at least 67% sequence identity or more with the nucleotide sequence of SEQ ID NO: 22;
(d) a fragment of a nucleotide sequence as defined in (a), (b) or (c) which is at least about 100 nucleotides in length;
(e) a sequence which is degenerate as a result of the genetic code to a sequence as defined in any one of (a), (b), (c) or (d);
(f) a nucleotide sequence which is the reverse complement of a nucleotide sequence as defined in (a), (b), (c), (d) or (e).

In another embodiment, the invention further provides a polypeptide having HMF/furfural oxidoreductase activity which comprises the amino acid sequence set out in SEQ ID NO: 25 or an amino acid sequence encoded by the nucleotide sequence of SEQ ID NO: 26 or a variant polypeptide thereof, wherein the variant has at least 45% sequence identity or more with the sequence set out in SEQ ID NO: 25.

In another embodiment, the invention further provides a polynucleotide which comprises:
(a) the nucleotide sequence set out in SEQ ID NO: 26;
(b) a nucleotide sequence which hybridizes selectively with a polynucleotide being the reverse complement of SEQ ID NO: 26;
(c) a nucleotide sequence having at least 66% sequence identity or more with the nucleotide sequence of SEQ ID NO: 26;
(d) a fragment of a nucleotide sequence as defined in (a), (b) or (c) which is at least about 100 nucleotides in length;
(e) a sequence which is degenerate as a result of the genetic code to a sequence as defined in any one of (a), (b), (c) or (d);
(f) a nucleotide sequence which is the reverse complement of a nucleotide sequence as defined in (a), (b), (c), (d) or (e).

In another embodiment, the invention further provides a polypeptide having LysR type transcriptional regulator activity which comprises the amino acid sequence set out in SEQ ID NO: 33 or an amino acid sequence encoded by the nucleotide sequence of SEQ ID NO: 34 or a variant polypeptide thereof, wherein the variant has at least 46% sequence identity or more with the sequence set out in SEQ ID NO: 33.

In another embodiment, the invention further provides a polynucleotide which comprises:
(a) the nucleotide sequence set out in SEQ ID NO: 34;
(b) a nucleotide sequence which hybridizes selectively with a polynucleotide being the reverse complement of SEQ ID NO: 34;
(c) a nucleotide sequence having at least 65% sequence identity or more with the nucleotide sequence of SEQ ID NO: 34;
(d) a fragment of a nucleotide sequence as defined in (a), (b) or (c) which is at least about 100 nucleotides in length;
(e) a sequence which is degenerate as a result of the genetic code to a sequence as defined in any one of (a), (b), (c) or (d);
(f) a nucleotide sequence which is the reverse complement of a nucleotide sequence as defined in (a), (b), (c), (d) or (e).

In another embodiment, the invention further provides a polypeptide having Furoyl-CoA dehydrogenase (large subunit) activity which comprises the amino acid sequence set out in SEQ ID NO: 35 or an amino acid sequence encoded by the nucleotide sequence of SEQ ID NO: 36 or a variant polypeptide thereof, wherein the variant has at least 54% sequence identity or more with the sequence set out in SEQ ID NO: 35.

In another embodiment, the invention further provides a polynucleotide which comprises:
(a) the nucleotide sequence set out in SEQ ID NO: 36;
(b) a nucleotide sequence which hybridizes selectively with a polynucleotide being the reverse complement of SEQ ID NO: 36;
(c) a nucleotide sequence having at least 66% sequence identity or more with the nucleotide sequence of SEQ ID NO: 36;
(d) a fragment of a nucleotide sequence as defined in (a), (b) or (c) which is at least about 100 nucleotides in length;
(e) a sequence which is degenerate as a result of the genetic code to a sequence as defined in any one of (a), (b), (c) or (d);
(f) a nucleotide sequence which is the reverse complement of a nucleotide sequence as defined in (a), (b), (c), (d) or (e).

In another embodiment, the invention further provides a polypeptide having Furoyl-CoA dehydrogenase (FAD binding subunit) activity which comprises the amino acid sequence set out in SEQ ID NO: 37 or an amino acid sequence encoded by the nucleotide sequence of SEQ ID NO: 38 or a variant polypeptide thereof, wherein the variant has at least 49% sequence identity or more with the sequence set out in SEQ ID NO: 37.

In another embodiment, the invention further provides a polynucleotide which comprises:
(a) the nucleotide sequence set out in SEQ ID NO: 38;
(b) a nucleotide sequence which hybridizes selectively with a polynucleotide being the reverse complement of SEQ ID NO: 38;
(c) a nucleotide sequence having at least 71% sequence identity or more with the nucleotide sequence of SEQ ID NO: 38;
(d) a fragment of a nucleotide sequence as defined in (a), (b) or (c) which is at least about 100 nucleotides in length;
(e) a sequence which is degenerate as a result of the genetic code to a sequence as defined in any one of (a), (b), (c) or (d);
(f) a nucleotide sequence which is the reverse complement of a nucleotide sequence as defined in (a), (b), (c), (d) or (e).

In another embodiment, the invention further provides a polypeptide having Furoyl-CoA dehydrogenase 2Fe-2S iron sulfur subunit activity which comprises the amino acid sequence set out in SEQ ID NO: 39 or an amino acid sequence encoded by the nucleotide sequence of SEQ ID NO: 40 or a variant polypeptide thereof, wherein the variant has at least 64% sequence identity or more with the sequence set out in SEQ ID NO: 39.

In another embodiment, the invention further provides a polynucleotide which comprises:
(a) the nucleotide sequence set out in SEQ ID NO: 40;
(b) a nucleotide sequence which hybridizes selectively with a polynucleotide being the reverse complement of SEQ ID NO: 40;
(c) a nucleotide sequence having at least 70% sequence identity or more with the nucleotide sequence of SEQ ID NO: 40; (d) a fragment of a nucleotide sequence as defined in (a), (b) or (c) which is at least about 100 nucleotides in length;
(e) a sequence which is degenerate as a result of the genetic code to a sequence as defined in any one of (a), (b), (c) or (d);
(f) a nucleotide sequence which is the reverse complement of a nucleotide sequence as defined in (a), (b), (c), (d) or (e).

In another embodiment, the invention further provides a polypeptide having Furoyl-CoA synthetase activity which comprises the amino acid sequence set out in SEQ ID NO: 41 or an amino acid sequence encoded by the nucleotide sequence of SEQ ID NO: 42 or a variant polypeptide thereof, wherein the variant has at least 57% sequence identity or more with the sequence set out in SEQ ID NO: 41.

In another embodiment, the invention further provides a polynucleotide which comprises:
(a) the nucleotide sequence set out in SEQ ID NO: 42;
(b) a nucleotide sequence which hybridizes selectively with a polynucleotide being the reverse complement of SEQ ID NO: 42;
(c) a nucleotide sequence having at least 68% sequence identity or more with the nucleotide sequence of SEQ ID NO: 42;
(d) a fragment of a nucleotide sequence as defined in (a), (b) or (c) which is at least about 100 nucleotides in length;
(e) a sequence which is degenerate as a result of the genetic code to a sequence as defined in any one of (a), (b), (c) or (d);
(f) a nucleotide sequence which is the reverse complement of a nucleotide sequence as defined in (a), (b), (c), (d) or (e).

In another embodiment, the invention further provides a polypeptide having 2-oxoglutaroyl-CoA hydrolase activity which comprises the amino acid sequence set out in SEQ ID NO: 43 or an amino acid sequence encoded by the nucleotide sequence of SEQ ID NO: 44 or a variant polypeptide thereof, wherein the variant has at least 72% sequence identity or more with the sequence set out in SEQ ID NO: 43.

In another embodiment, the invention further provides a polynucleotide which comprises:
(a) the nucleotide sequence set out in SEQ ID NO: 44;
(b) a nucleotide sequence which hybridizes selectively with a polynucleotide being the reverse complement of SEQ ID NO: 44;
(c) a nucleotide sequence having at least 74% sequence identity or more with the nucleotide sequence of SEQ ID NO: 44;
(d) a fragment of a nucleotide sequence as defined in (a), (b) or (c) which is at least about 100 nucleotides in length;
(e) a sequence which is degenerate as a result of the genetic code to a sequence as defined in any one of (a), (b), (c) or (d);
(f) a nucleotide sequence which is the reverse complement of a nucleotide sequence as defined in (a), (b), (c), (d) or (e).

In yet another embodiment, the invention further provides vectors incorporating polynucleotide sequences as described above or cells comprising polypeptide sequences as described above.

In another embodiment, the invention further provides host microorganism cells transformed or transfected by polynucleotide sequences as described above or by vectors as described above under conditions conducive to express one or more of a Furoyl-CoA dehydrogenase, a Furoyl-CoA synthetase, a 2-oxoglutaroyl-CoA hydrolase, a 2,5-furan-dicarboxylic acid decarboxylase 1, a 2,5-furan-dicarboxylic acid decarboxylase 2 and a HMF/furfural oxidoreductase.

In another embodiment, the invention further provides a process for the in-situ detoxification of lignocellulose hydrolysate containing furanic compounds, such as preferably one or more of HMF, Furfurylalcohol, Furfural and/or Furoic acid, with a suitable host microorganism, comprising contacting the lignocellulose hydrolysate with the host microorganism under conditions facilitating the expression of one or more of a Furoyl-CoA dehydrogenase, a Furoyl-CoA synthetase, a 2-oxoglutaroyl-CoA hydrolase, a 2,5-furan-dicarboxylic acid decarboxylase 1, a 2,5-furan-dicarboxylic acid decarboxylase 2 and a HMF/furfural oxidoreductase from a microorganism from the family of Burkholderiaceae to convert furanic compounds, such as HMF, Furfurylalcohol, Furfural and/or Furoic acid, to non-toxic components to obtain a detoxified lignocellulose hydrolysate.

In another embodiment, the invention further provides a process for the production of Furoyl-CoA dehydrogenase, Furoyl-CoA synthetase, 2-oxoglutaroyl-CoA hydrolase, 2,5-furan-dicarboxylic acid decarboxylase 1,2,5-furan-dicarboxylic acid decarboxylase 2 and/or a HMF/furfural oxidoreductase which are at least 45% identical to those expressed by Cupriavidus basilensis HMF14 DSM 22875, or a host microorganism as described above, comprising
(a) culturing a microorganism in a nutrient medium containing carbon and nitrogen sources and inorganic salts; and
(b) isolating the enzymes produced from the microorganism.

In another embodiment, the invention further provides a process for the conversion of 5-hydroxymethylfurfural (HMF), 2,5-dihydroxymethyl furan (HMF alcohol), 5-hydroxymethyl-2-furancarboxylic acid (HMF acid) and/or 2,5-furandicarboxylic acid to 2-furoyl CoA, comprising comprising contacting furfuryl alcohol and/or furfural with a furoyl-CoA dehydrogenase, furoyl-CoA synthetase, 2-oxoglutaroyl-CoA hydrolase, and 2,5-furan-dicarboxylic acid decarboxylase catalyst in the presence of one or more coenzyme cofactor.

### BRIEF DESCRIPTION OF THE DRAWINGS

FIG. 1 shows the growth of C. basilensis HMF14 on mineral salts medium with furfural as the sole carbon source.

FIG. 2 illustrates the growth of C. basilensis HMF14 on different concentrations of furfural (A) or HMF (B).

FIG. 3 depicts the detection of PHA in cultures of C. basilensis HMF14 in minimal medium with 120 mM acetate.

FIG. 4 illustrates the detoxification of lignocellulosic hydrolysate by C. basilensis HMF14.

FIG. 5 illustrates a reaction scheme of reactions catalysed by Cupriavidus basilensis HMF 14.

FIG. 6 is a schematic representation of the genetic organization of the furfural and HMF metabolic genes in C. basilensis HMF14 (A) and other species (B) that were identified as potential furfural and/or HMF utilizers.

### DETAILED DESCRIPTION OF THE INVENTION

### Description of the Sequences

Sequence Listing submitted in computer readable form submitted electronically herewith is hereby incorporated by reference.

Sequences SEQ ID NO: 1 to 14 set out the DNA sequences of several synthetic DNA primers used for PCR and/or for cloning or isolation of relevant genes. Underlined sequences indicate a restriction site.

### Synthetic DNA

SEQ ID NO: 1
Hybridizes to hmfA, forward primer.

GCACGCGCCTGAGTTACGAC

### Synthetic DNA

SEQ ID NO: 2
Hybridizes to hmfR2, forward primer.

CATGCTCGGCGCTGGTGAC

### Synthetic DNA

SEQ ID NO: 3
Hybridizes near start site of hmfF gene, forward primer.

CATGAATTCCGACCCAGGAGTCACGCCAT

### Synthetic DNA

SEQ ID NO: 4
Hybridizes near stop site of hmfF gene, reverse primer.

CGGCGGCCGCGGATATACCGACAATGATGCGTCTCT

### Synthetic DNA

SEQ ID NO: 5
Hybridizes near stop site of hmfG gene, reverse primer.

CGGCGGCCGCTGTCTCCTGCCTGTTCAGCATTCA

### Synthetic DNA

SEQ ID NO: 6
Hybridizes near stop site of hmfD gene, reverse primer.

GCGGGCCCCTTACTCCTTGATGGTATCGACAGG

### Synthetic DNA

SEQ ID NO: 7
Hybridizes near start site of hmfA gene, forward primer.

GCGGTACCGGGAGGGCCGGTCATGAG

### Synthetic DNA

SEQ ID NO: 8
Hybridizes near start site of hmfD gene, forward primer.

GCGGTACCGGCGTAGATACCCAGGAGGC

### Synthetic DNA

SEQ ID NO: 9
Hybridizes near stop site of hmfC gene, reverse primer.

GCGGGCCCCCACGCTTTGCAGGAAGGTG

### SEQ ID NO: 10

Hybridizes near start site of hmfG gene, forward primer.

CGGAATTCCGGCGCATGTGTTCACGC

### Synthetic DNA

SEQ ID NO: 10

CGGAATTCCGGCGCATGTGTTCACGC

### Synthetic DNA

SEQ ID NO: 11
Hybridizes near stop site of hmfE gene, reverse primer.

GCGCGGCCGCCGCCCGCGATTTCCATCAG

### Synthetic DNA

SEQ ID NO: 12
Hybridizes near start site of hmfE gene, forward primer.

GCGGTACCCCATCAAGGAGTAAGACATGACCC

### Synthetic DNA

SEQ ID NO: 13
Hybridizes near start site of hmfH gene, forward primer.

CGGAATTCCACATGACAAGGGGAGACCG

### Synthetic DNA

SEQ ID NO: 14
Hybridizes near stop site of hmfH gene, reverse primer.

CGGAATTCGCTTCGGTCTTCAACTCGGATG

SEQ ID NO: 15 sets out the amino acid sequence of a putative furan aldehyde dehydrogenase (adh) of Cupriavidus basilensis strain HMF14.
SEQ ID NO: 16 sets out the coding sequence of a putative furan aldehyde dehydrogenase (adh).
SEQ ID NO: 17 sets out the amino acid sequence of LysR-type transcriptional regulator hmfR1.
SEQ ID NO: 18 sets out the coding sequence of hmfR1.
SEQ ID NO: 19 sets out the amino acid sequence of 5-hydroxymethyl-2-furoic acid decarboxylase 1 hmfF.
SEQ ID NO: 20 sets out the coding sequence of hmfF.
SEQ ID NO: 21 sets out the amino acid sequence of 5-hydroxymethyl-2-furoic acid decarboxylase 2 hmfG.
SEQ ID NO: 22 sets out the coding sequence of hmfG.
SEQ ID NO: 23 sets out sets out the amino acid sequence of the extracytoplasmic solute receptor hmfH′.
SEQ ID NO: 24 sets out the coding sequence of hmfH′.
SEQ ID NO: 25 sets out the amino acid sequence of HMF/furfural oxidoreductase hmfH.
SEQ ID NO: 26 sets out the coding sequence of hmfH.
SEQ ID NO: 27 sets out the amino acid sequence of fatty acid hydroxylase hyd.
SEQ ID NO: 28 sets out the coding sequence of hyd.
SEQ ID NO: 29 sets out the amino acid sequence of truncated LysR-type transcriptional regulator LysR hmfRt.
SEQ ID NO: 30 sets out the coding sequence for LysR hmfRt.
SEQ ID NO: 31 sets out the amino acid sequence for major facilitator superfamily transportera putative furanic MFS-type transporter mfs1.
SEQ ID NO: 32 sets out the coding sequence for a putative furanic MFS-type transporter major facilitator superfamily transporter mfs1.
SEQ ID NO: 33 sets out the amino acid sequence for LysR-type regulator hmfR2.
SEQ ID NO: 34 sets out the coding sequence for LysR-type regulator hmfR2.
SEQ ID NO: 35 sets out the amino acid sequence for the furoyl-CoA dehydrogenase large subunit hmfA.
SEQ ID NO: 36 sets out the coding sequence for hmfA.
SEQ ID NO: 37 sets out the amino acid sequence for the Furoyl-CoA dehydrogenase FAD binding subunit, hmfB.
SEQ ID NO: 38 sets out the coding sequence for hmfB.
SEQ ID NO: 39 sets out the amino acid sequence for the Furoyl-CoA dehydrogenase 2Fe-2S iron sulfur subunit, hmfC
SEQ ID NO: 40 sets out the coding sequence for hmfC.
SEQ ID NO: 41 sets out the amino acid sequence for the Furoyl-CoA synthetase hmfD.
SEQ ID NO: 42 sets out the coding sequence for hmfD.
SEQ ID NO: 43 sets out the amino acid sequence for the 2-oxoglutaroyl-CoA hydrolase hmfE.
SEQ ID NO: 44 sets out the coding sequence for hmfE.
SEQ ID NO: 45 sets out the amino acid sequence for a putative furanic MFS-type transporter the major facilitator superfamily transporter mfs2.
SEQ ID NO: 46 sets out the coding sequence for a putative furanic MFS-type transporter mfs2.

### Definition of Terms and Description

The following terms will be understood as defined herein unless otherwise stated. Such definitions include without recitation those meanings associated with these terms known to those skilled in the art.

Within the context of the subject invention, the term “furanic compound” may be selected from one or more of the following compounds: Furfural, furfurylalcohol, furoic acid, hydroxymethylfurfural, hydroxymethylfurancarboxylic acid, furandimethanol, diformylfuran, formylfuran carboxylic acid, furandicarboxylic acid, formylthiophene, thiophene methanol, thiophene carboxylic acid, hydroxymethyl formylthiophene, hydroxymethyl thiopene carboxylic acid, thiophene dimethanol, diformyl thiophene, formylthiopene carboxylic acid, thiophene dicarboxylic acid, formylpyrrole, pyrrolylmethanol, pyrrole carboxylic acid, hydroxymethyl formylpyrrole, hydroxymethyl pyrrole carboxylic acid, pyrroledimethanol, diformylpyrrole, formylpyrrole carboxylic acid and pyrrole dicarboxylic acid.

Preferred furanic compounds are furfural, furfurylalcohol, furoic acid, hydroxymethylfurfural, hydroxymethylfurancarboxylic acid, furandimethanol, diformylfuran, formylfuran carboxylic acid, furandicarboxylic acid, more preferably at least one of hydroxymethylfurfural (HMF), hydroxymethylfuran carboxylic acid (HMF acid), 2,5-dihydroxymethylfuran (HMF alcohol). At the furanic compound, the furan ring or any or its substitutable side-group may be substituted, e.g. with OH, alkyl (e.g. C1-C10 alkyl), allyl, aryl or RO-ether moiety, including cyclic groups, in the furan ring on any available position. More preferably, Furanic compounds are herein understood to be any compound having a furan group that may be oxidized to 2,5-furan-dicarboxylic acid or a precursor thereof.

Expression of polypeptides according to the invention: Regardless of the exact mechanism utilized for expression of enzymes, it is contemplated that such expression is transferable by the introduction of genes encoding these enzymes into another host cell by methods known in the art. Genetic elements as herein defined include nucleic acids (generally DNA or RNA) having expressible coding sequences for products such as proteins, specifically enzymes, apoproteins or antisense RNA, which express or regulate expression of relevant enzymes. The expressed proteins can function as enzymes, repress or derepress enzyme activity or control expression of enzymes. Recombinant DNA encoding these expressible sequences can be either chromosomal (integrated into the host cell chromosome by, for example, homologous recombination) or extra-chromosomal (for example, carried by one or more plasmids, cosmids and other vectors capable of self replication). It is understood that the recombinant DNA utilized for transforming the host cell in accordance with this invention can include, in addition to structural genes and transcription factors, expression control sequences, including promoters, repressors and enhancers, that act to control expression or derepression of coding sequences for proteins, apoproteins or antisense RNA. For example, such control sequences can be inserted into wild-type host cells to promote overexpression of selected enzymes already encoded in the host cell genome, or alternatively they can be used to control synthesis of extrachromosomally encoded enzymes.

Recombinant DNA can be introduced into the host cell by any means, including, but not limited to, plasmids, cosmids, phages, yeast artificial chromosomes or other vectors that mediate transfer of genetic elements into a host cell. These vectors can include an origin of replication, along with cis-acting control elements that control replication of the vector and the genetic elements carried by the vector. Selectable markers can be present on the vector to aid in the identification of host cells into which genetic elements have been introduced.

Means for introducing genetic elements into a host cell (e.g. cloning) are well known to the skilled artisan. One can utilize an extrachromosomal multi-copy plasmid vector to insert the genetic elements in accordance with the present invention. Plasmid-borne introduction of the genetic element into host cells involves an initial cleaving of a plasmid vector with a restriction enzyme, followed by ligation of the plasmid and genetic elements encoding for the targeted enzyme species in accordance with the invention. Upon recircularization of the ligated recombinant plasmid, infection (e.g., packaging in phage lambda) or other mechanism for plasmid transfer (e.g., electroporation, microinjection, etc.) is utilized to transfer the plasmid into the host cell. Plasmids suitable for insertion of genetic elements into the host cell are well known to the skilled artisan. Other gene cloning methods include, but are not limited to, direct integration of the genetic material into the chromosome. This can occur by a variety of means, including cloning the genetic elements described herein on non-replicating plasmids flanked by homologous DNA sequences of the host chromosome; upon transforming said recombinant plasmid into a host the genetic elements can be introduced into the chromosome by DNA recombination. Such recombinant strains can be recovered if the integrating DNA fragments contain a selectable marker, such as antibiotic resistance. Alternatively, the genetic elements can be directly introduced into the chromosome of a host cell without use of a non-replicating plasmid. This can be done by synthetically producing DNA fragments of the genetic elements in accordance to the present invention that also contain homologous DNA sequences of the host chromosome. Again if these synthetic DNA fragments also contain a selectable marker, the genetic elements can be inserted into the host chromosome.

A DNA fragment, as used herein, may encode regulatory and/or structural genetic information. A DNA fragment useful in the present invention shall also include: nucleic acid molecules encoding sequences complementary to those provided; nucleic acid molecules (DNA or RNA) which hybridize under stringent conditions to those molecules that are provided; or those nucleic acid molecules that, but for the degeneracy of the genetic code, would hybridize to the molecules provided or their complementary strands. “Stringent” hybridization conditions are those that minimize formation of double stranded nucleic acid hybrids from non-complementary or mismatched single stranded nucleic acids. In addition, hybridization stringency may be affected by the various components of the hybridization reaction, including salt concentration, the presence or absence of formamide, the nucleotide composition of the nucleic acid molecules, etc. The nucleic acid molecules useful in the present invention may be either naturally or synthetically derived.

A “heterologous” or “exogenous” DNA fragment has been introduced into the host microorganism by any process such as transformation, transfection, transduction, conjugation, electroporation, etc. Additionally, it should be noted that it is possible that the host cell into which the “heterologous” DNA fragment has been inserted may itself also naturally harbour molecules encoding the same or similar sequences. A molecule such as this is referred to as a “homologous” DNA molecule.

A stably transformed microorganism is one that has had one or more DNA fragments introduced such that the introduced molecules are maintained, replicated and segregated in a growing culture. Stable transformation may be due to multiple or single chromosomal integration (s) or by (an) extrachromosomal element(s) such as (a) plasmid vector(s). A plasmid vector is capable of directing the expression of polypeptides encoded by particular DNA fragments.

Expression may be constitutive or regulated by inducible (or repressible) promoters that enable high levels of transcription of functionally associated DNA fragments encoding specific polypeptides.

Regardless of the exact mechanism utilized for expression of enzymes necessary for detoxification of lignocelluloses, it is contemplated that such expression is transferable by the introduction of genes encoding these enzymes into another host cell by methods known in the art. Genetic elements as herein defined include nucleic acids (generally DNA or RNA) having expressible coding sequences for products such as proteins, specifically enzymes, apoproteins or antisense RNA, which express or regulate expression of relevant enzymes. The expressed proteins can function as enzymes, repress or derepress enzyme activity or control expression of enzymes. Recombinant DNA encoding these expressible sequences can be either chromosomal (integrated into the host cell chromosome by, for example, homologous recombination) or extra-chromosomal (for example, carried by one or more plasmids, cosmids and other vectors capable of self replication). It is understood that the recombinant DNA utilized for transforming the host cell in accordance with this invention can include, in addition to structural genes and transcription factors, expression control sequences, including promoters, repressors and enhancers that act to control expression or derepression of coding sequences for proteins, apoproteins or antisense RNA. For example, such control sequences can be inserted into wild-type host cells to promote overexpression of selected enzymes already encoded in the host cell genome, or alternatively they can be used to control synthesis of extrachromosomally encoded enzymes.

Recombinant DNA can be introduced into the host cell by any means, including, but not limited to, plasmids, cosmids, phages, yeast artificial chromosomes or other vectors that mediate transfer of genetic elements into a host cell. These vectors can include an origin of replication, along with cis-acting control elements that control replication of the vector and the genetic elements carried by the vector. Selectable markers can be present on the vector to aid in the identification of host cells into which genetic elements have been introduced

Means for introducing genetic elements into a host cell (e.g. cloning) are well known to the skilled artisan. One can utilize an extrachromosomal multi-copy plasmid vector to insert the genetic elements in accordance with the present invention. Plasmid-borne introduction of the genetic element into host cells involves an initial cleaving of a plasmid vector with a restriction enzyme, followed by ligation of the plasmid and genetic elements encoding for the targeted enzyme species in accordance with the invention. Upon recircularization of the ligated recombinant plasmid, infection (e.g., packaging in phage lambda) or other mechanism for plasmid transfer (e.g., electroporation, microinjection, etc.) is utilized to transfer the plasmid into the host cell. Plasmids suitable for insertion of genetic elements into the host cell are well known to the skilled artisan.

Other gene cloning methods include, but are not limited to, direct integration of the genetic material into the chromosome. This can occur by a variety of means, including cloning the genetic elements described herein on non-replicating plasmids flanked by homologous DNA sequences of the host chromosome; upon transforming said recombinant plasmid into a host the genetic elements can be introduced into the chromosome by DNA recombination. Such recombinant strains can be recovered if the integrating DNA fragments contain a selectable marker, such as antibiotic resistance. Alternatively, the genetic elements can be directly introduced into the chromosome of a host cell without use of a non-replicating plasmid. This can be done by synthetically producing DNA fragments of the genetic elements in accordance to the present invention that also contain homologous DNA sequences of the host chromosome. Again if these synthetic DNA fragments also contain a selectable marker, the genetic elements can be inserted into the host chromosome.

A preferred embodiment of the invention is a host cell comprising one or more polypeptides, polynucleotides, nucleic acid constructs or vectors according to the invention. This may be a cell in which the polypeptides, polynucleotides, nucleic acid constructs or vectors can suitably be expressed. The enzymes according to the invention may be favourably expressed in a host cell. The host cell according to the invention may be any host cell. The cell may be a prokaryote cell, an eukaryote cell, a plant cell or an animal cell.

The cell may be a host microorganism, which may be an autonomous single-celled organism useful for microbial production of biofuels, such as ethanol, as well as production of chemicals, including both eukaryotic and prokaryotic microorganisms. Useful microorganisms may be prokaryotes or eukaryotes and include organisms like bacteria, yeast, and fungi and plants.

Such a host microorganism usually contains all DNA, either endogenous or heterologous, required for the digestion of furanic compounds from lignocellulose hydrolysate. It may further preferably also comprise all DNA, either endogenous or heterologous, required for the conversion of fermentable sugars from lignocellulose hydrolysate for the production of a biofuel component such as for instance ethanol, n- or iso-butanol from lignocelluloses hydrosylate. In such cell one or more gene may be deleted, knocked-out or disrupted in full or in part. According to an embodiment, the host cell according to the invention is a eukaryotic host cell. Preferably, the eukaryotic cell is a mammalian, insect, plant, fungal, or algal cell. Preferred mammalian cells include e.g. Chinese hamster ovary (CHO) cells, COS cells, 293 cells, PerC6 cells, and hybridomas. Preferred insect cells include e.g. Sf9 and Sf21 cells and derivatives thereof.

More preferably, the eukaryotic cell is a fungal cell, such as for instance a yeast cell, such as those of the Candida, Hansenula, Kluyveromyces, Pichia, Saccharomyces, Schizosaccharomyces, and/or Yarrowia strains. More preferably, it si a cell selected from Kluyveromyces lactis, S. cerevisiae, Hansenula polymorpha, Yarrowia lipolytica and Pichia pastoris, or a filamentous fungal cell. More preferably, the eukaryotic cell is a yeast cell. “Filamentous fungi” include all filamentous forms of the subdivision Eumycota and Oomycota (as defined by Hawksworth et al., In, Ainsworth and Bisby's Dictionary of The Fungi, 8th edition, 1995, CAB International, University Press, Cambridge, UK). The filamentous fungi are characterized by a mycelial wall composed of chitin, cellulose, glucan, chitosan, mannan, and other complex polysaccharides. Vegetative growth is by hyphal elongation and carbon catabolism is obligately aerobic. Filamentous fungal strains include, but are not limited to, strains of Acremonium, Agaricus, Aspergillus, Aureobasidium, Chrysosporium, Coprinus, Cryptococcus, Filibasidium, Fusarium, Humicola, Magnaporthe, Mucor, Myceliophthora, Neocallimastix, Neurospora, Paecilomyces, Penicillium, Piromyces, Panerochaete, Pleurotus, Schizophyllum, Talaromyces, Thermoascus, Thielavia, Tolypocladium, and Trichoderma.

Preferred filamentous fungal cells belong to a species of an Aspergillus, Chrysosporium, Penicillium, Talaromyces or Trichoderma genus, and most preferably a species of Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum. When the host cell according to the invention is an Aspergillus host cell, the host cell preferably is CBS 513.88, CBS124.903 or a derivative thereof. Several strains of filamentous fungi are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL) Aspergillus niger CBS 513.88, Aspergillus oryzae ATCC 20423, IFO 4177, ATCC 1011, ATCC 9576, ATCC14488-14491, ATCC 11601, ATCC12892, P. chrysogenum CBS 455.95, Penicillium citrinum ATCC 38065, Penicillium chrysogenum P2, Talaromyces emersonii CBS 124.902, Acremonium chrysogenum ATCC 36225 or ATCC 48272, Trichoderma reesei ATCC 26921 or ATCC 56765 or ATCC 26921, Aspergillus sojae ATCC11906, Chrysosporium lucknowense ATCC44006 and derivatives thereof.

According to another preferred embodiment, the host cell according to the invention is a prokaryotic cell. Preferably, the prokaryotic host cell is bacterial cell. The term “bacterial cell” includes both Gram-negative and Gram-positive microorganisms. Suitable bacteria may be selected from e.g. Escherichia, Anabaena, Caulobacter, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces. Preferably, the bacterial cell is selected from the group consisting of B. subtilis, B. amyloliquefaciens, B. licheniformis, B. puntis, B. megaterium, B. halodurans, B. pumilus, G. (Gluconobacter) oxydans, Caulobacter crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter.

Preferred host organisms are any genus/species that are able to produce and incorporate the molydopterin cofactor required for the furoyl-CoA dehydrogenase, or comprising a molydopterin cofactor independent furoyl-CoA dehydrogenase, flavin adenine dinucleotide (FAD) or nicotine adenine dehydrogenase (NAD+).

For specific uses of a compound produced in a host cell according to the invention, the selection of the host cell may be made according to such use. Where e.g. the compound produced in a host cell according to the invention is to be used in food applications, a host cell may be selected from a food-grade organism such as Saccharomyces cerevisiae. Specific uses include, but are not limited to, food, (animal) feed, pharmaceutical, agricultural such as crop-protection, and/or personal care applications.

The invention further relates to method for the preparation of polypeptides having various enzymatic activities. This method comprises cultivating a cell according to the invention under conditions which allow for expression of the appropriate polypeptide and, optionally, recovering the expressed polypeptide and to a polypeptide obtainable by that method.

A polypeptide according to the invention having aldehyde dehydrogenase activity comprises the amino acid sequence set out in SEQ ID NO: 15 or an amino acid sequence encoded by the nucleotide sequence of SEQ ID NO: 16 or a variant polypeptide thereof, wherein the variant has at least 41% sequence identity or more with the sequence set out in SEQ ID NO: 15.

In one embodiment the variant nucleic acid molecule comprises a nucleotide sequence encoding a protein, wherein the variant nucleic acid molecule comprises a substantially homologous nucleotide sequence of at least 64%, 66%, 68%, 70%, 72%, 75%, 80%, 85%, 90%, 91%, 92%, 93%, 94%, 95%, 96%, 97%, 98%, 99% or more homologous to the nucleotide sequence shown in SEQ ID NO: 16.

In another embodiment the variant protein comprises a substantially homologous amino acid sequence of at least 41%, 45%, 47%, 50%, 55%, 60%, 65%, 70%, 75%, 80%, 85%, 90%, 91%, 92%, 93%, 94%, 95%, 96%, 97%, 98%, 99% or more homologous to the amino acid sequence shown in SEQ ID NO: 15.

An embodiment of the invention is a polynucleotide which comprises:
(a) the nucleotide sequence set out in SEQ ID NO: 16;
(b) a nucleotide sequence which hybridizes selectively with a polynucleotide being the reverse complement of SEQ ID NO: 16; (c) a nucleotide sequence having at least 64% sequence identity or more with the nucleotide sequence of SEQ ID NO: 16; (d) a fragment of a nucleotide sequence as defined in (a), (b) or (c) which is at least about 100 nucleotides in length; (e) a sequence which is degenerate as a result of the genetic code to a sequence as defined in any one of (a), (b), (c) or (d);
(f) a nucleotide sequence which is the reverse complement of a nucleotide sequence as defined in (a), (b), (c), (d) or (e).

A polypeptide according to the invention having LysR family transcriptional regulator activity comprises the amino acid sequence set out in SEQ ID NO: 17 or an amino acid sequence encoded by the nucleotide sequence of SEQ ID NO: 18 or a variant polypeptide thereof, wherein the variant has at least 47% sequence identity or more with the sequence set out in SEQ ID NO: 17.

In one embodiment the variant nucleic acid molecule comprises a nucleotide sequence encoding a protein, wherein the variant nucleic acid molecule comprises a substantially homologous nucleotide sequence of at least 72%, 75%, 80%, 85%, 90%, 91%, 92%, 93%, 94%, 95%, 96%, 97%, 98%, 99% or more homologous to the nucleotide sequence shown in SEQ ID NO: 18.

In another embodiment the variant protein comprises a substantially homologous amino acid sequence of at least 47%, 50%, 55%, 60%, 65%, 70%, 75%, 80%, 85%, 90%, 91%, 92%, 93%, 94%, 95%, 96%, 97%, 98%, 99% or more homologous to the amino acid sequence shown in SEQ ID NO: 17.

An embodiment of the invention is a polynucleotide which comprises: (a) the nucleotide sequence set out in SEQ ID NO: 18; (b) a nucleotide sequence which hybridizes selectively with a polynucleotide being the reverse complement of SEQ ID NO: 18; (c) a nucleotide sequence having at least 72% sequence identity or more with the nucleotide sequence of SEQ ID NO: 18; (d) a fragment of a nucleotide sequence as defined in (a), (b) or (c) which is at least about 100 nucleotides in length; (e) a sequence which is degenerate as a result of the genetic code to a sequence as defined in any one of (a), (b), (c) or (d); (f) a nucleotide sequence which is the reverse complement of a nucleotide sequence as defined in (a), (b), (c), (d) or (e), or encodes a polypeptide; and to the relating polypeptides.

A polypeptide according to the invention having 2,5-furan-dicarboxylic acid decarboxylase 1 activity comprises the amino acid sequence set out in SEQ ID NO: 19 or an amino acid sequence encoded by the nucleotide sequence of SEQ ID NO: 20 or a variant polypeptide thereof, wherein the variant has at least 54% sequence identity or more with the sequence set out in SEQ ID NO: 19.

An embodiment of the invention is a polynucleotide which comprises: (a) the nucleotide sequence set out in SEQ ID NO: 20; (b) a nucleotide sequence which hybridizes selectively with a polynucleotide being the reverse complement of SEQ ID NO: 20; (c) a nucleotide sequence having at least 66% sequence identity or more with the nucleotide sequence of SEQ ID NO: 20; (d) a fragment of a nucleotide sequence as defined in (a), (b) or (c) which is at least about 100 nucleotides in length; (e) a sequence which is degenerate as a result of the genetic code to a sequence as defined in any one of (a), (b), (c) or (d); (f)a nucleotide sequence which is the reverse complement of a nucleotide sequence as defined in (a), (b), (c), (d) or (e).

In one embodiment the variant nucleic acid molecule comprises a nucleotide sequence encoding a protein, wherein the variant nucleic acid molecule comprises a substantially homologous nucleotide sequence of at least 66%, 70%, 75%, 80%, 85%, 90%, 91%, 92%, 93%, 94%, 95%, 96%, 97%, 98%, 99% or more homologous to the nucleotide sequence shown in SEQ ID NO: 20.

In another embodiment the variant protein comprises a substantially homologous amino acid sequence of at least 54%, 55%, 60%, 65%, 70%, 75%, 80%, 85%, 90%, 91%, 92%, 93%, 94%, 95%, 96%, 97%, 98%, 99% or more homologous to the amino acid sequence shown in SEQ ID NO: 19.

A polypeptide according to the invention having 2,5-furan-dicarboxylic acid decarboxylase 2 activity comprises the amino acid sequence set out in SEQ ID NO: 21 or an amino acid sequence encoded by the nucleotide sequence of SEQ ID NO: 22 or a variant polypeptide thereof, wherein the variant has at least 52% sequence identity or more with the sequence set out in SEQ ID NO: 21.

An embodiment of the invention is a polynucleotide which comprises: (a) the nucleotide sequence set out in SEQ ID NO: 22; (b) a nucleotide sequence which hybridizes selectively with a polynucleotide being the reverse complement of SEQ ID NO: 22; (c) a nucleotide sequence having at least 67% sequence identity or more with the nucleotide sequence of SEQ ID NO: 22; (d) a fragment of a nucleotide sequence as defined in (a), (b) or (c) which is at least about 100 nucleotides in length;
(e) a sequence which is degenerate as a result of the genetic code to a sequence as defined in any one of (a), (b), (c) or (d); (f) a nucleotide sequence which is the reverse complement of a nucleotide sequence as defined in (a), (b), (c), (d) or (e).

In one embodiment the variant nucleic acid molecule comprises a nucleotide sequence encoding a protein, wherein the variant nucleic acid molecule comprises a substantially homologous nucleotide sequence of at least 67%, 70%, 75%, 80%, 85%, 90%, 91%, 92%, 93%, 94%, 95%, 96%, 97%, 98%, 99% or more homologous to the nucleotide sequence shown in SEQ ID NO: 22.

In another embodiment the variant protein comprises a substantially homologous amino acid sequence of at least 52%, 55%, 60%, 65%, 70%, 75%, 80%, 85%, 90%, 91%, 92%, 93%, 94%, 95%, 96%, 97%, 98%, 99% or more homologous to the amino acid sequence shown in SEQ ID NO: 21.

A polypeptide according to the invention having HMF/furfural oxidoreductase activity comprises the amino acid sequence set out in SEQ ID NO: 25 or an amino acid sequence encoded by the nucleotide sequence of SEQ ID NO: 26 or a variant polypeptide thereof, wherein the variant has at least 45% sequence identity or more with the sequence set out in SEQ ID NO: 25.

An embodiment of the invention is a polynucleotide which comprises: (a) the nucleotide sequence set out in SEQ ID NO: 26; (b) a nucleotide sequence which hybridizes selectively with a polynucleotide being the reverse complement of SEQ ID NO: 26; (c) a nucleotide sequence having at least 66% sequence identity or more with the nucleotide sequence of SEQ ID NO: 26; (d) a fragment of a nucleotide sequence as defined in (a), (b) or (c) which is at least about 100 nucleotides in length; (e) a sequence which is degenerate as a result of the genetic code to a sequence as defined in any one of (a), (b), (c) or (d); (f) a nucleotide sequence which is the reverse complement of a nucleotide sequence as defined in (a), (b), (c), (d) or (e).

In one embodiment the variant nucleic acid molecule comprises a nucleotide sequence encoding a protein, wherein the variant nucleic acid molecule comprises a substantially homologous nucleotide sequence of at least 66%, 70%, 75%, 80%, 85%, 90%, 91%, 92%, 93%, 94%, 95%, 96%, 97%, 98%, 99% or more homologous to the nucleotide sequence shown in SEQ ID NO: 26.

In another embodiment the variant protein comprises a substantially homologous amino acid sequence of at least 45%, 50%, 55%, 60%, 65%, 70%, 75%, 80%, 85%, 90%, 91%, 92%, 93%, 94%, 95%, 96%, 97%, 98%, 99% or more homologous to the amino acid sequence shown in SEQ ID NO: 25.

A polypeptide according to the invention having fatty acid hydroxylase (hyd) activity comprises the amino acid sequence set out in SEQ ID NO: 27, or an amino acid sequence encoded by the nucleotide sequence of SEQ ID NO: 28 or a variant polypeptide thereof, wherein the variant has at least 66% sequence identity or more with the sequence set out in SEQ ID NO: 28.

An embodiment of the invention is a polynucleotide which comprises: (a) the nucleotide sequence set out in SEQ ID NO: 28; (b) a nucleotide sequence which hybridizes selectively with a polynucleotide being the reverse complement of SEQ ID NO: 28; (c) a nucleotide sequence having at least 66% sequence identity or more with the nucleotide sequence of SEQ ID NO: 28; (d) a fragment of a nucleotide sequence as defined in (a), (b) or (c) which is at least about 100 nucleotides in length; (e) a sequence which is degenerate as a result of the genetic code to a sequence as defined in any one of (a), (b), (c) or (d); (f) a nucleotide sequence which is the reverse complement of a nucleotide sequence as defined in (a), (b), (c), (d) or (e).

In one embodiment the variant nucleic acid molecule comprises a nucleotide sequence encoding a protein, wherein the variant nucleic acid molecule comprises a substantially homologous nucleotide sequence of at least 66%, 70%, 75%, 80%, 85%, 90%, 91%, 92%, 93%, 94%, 95%, 96%, 97%, 98%, 99% or more homologous to the nucleotide sequence shown in SEQ ID NO: 28.

In another embodiment the variant protein comprises a gsubstantially homologous amino acid sequence of at least 31%, 33%, 35%, 40%, 45%, 50%, 55%, 60%, 65%, 70%, 75%, 80%, 85%, 90%, 91%, 92%, 93%, 94%, 95%, 96%, 97%, 98%, 99% or more homologous to the amino acid sequence shown in SEQ ID NO: 27.

A polypeptide according to the invention having truncated LysR-type transcriptional regulator LysR (hmfRt) activity activity comprises the amino acid sequence set out in SEQ ID NO: 29, or an amino acid sequence encoded by the nucleotide sequence of SEQ ID NO: 30 or a variant polypeptide thereof, wherein the variant has at least 65% sequence identity or more with the sequence set out in SEQ ID NO: 30.

An embodiment of the invention is a polynucleotide which comprises: (a) the nucleotide sequence set out in SEQ ID NO: 30; (b) a nucleotide sequence which hybridizes selectively with a polynucleotide being the reverse complement of SEQ ID NO: 30; (c) a nucleotide sequence having at least 85% sequence identity or more with the nucleotide sequence of SEQ ID NO: 30; (d) a fragment of a nucleotide sequence as defined in (a), (b) or (c) which is at least about 100 nucleotides in length; (e) a sequence which is degenerate as a result of the genetic code to a sequence as defined in any one of (a), (b), (c) or (d); (f) a nucleotide sequence which is the reverse complement of a nucleotide sequence as defined in (a), (b), (c), (d) or (e).

In one embodiment the variant nucleic acid molecule comprises a nucleotide sequence encoding a protein, wherein the variant nucleic acid molecule comprises a substantially homologous nucleotide sequence of at least 85%, 88%, 90%, 91%, 92%, 93%, 94%, 95%, 96%, 97%, 98%, 99% or more homologous to the nucleotide sequence shown in SEQ ID NO: 30.

In another embodiment the variant protein comprises a substantially homologous amino acid sequence of at least 37%, 40%, 45%, 50%, 55%, 60%, 65%, 70%, 75%, 80%, 85%, 90%, 91%, 92%, 93%, 94%, 95%, 96%, 97%, 98%, 99% or more homologous to the amino acid sequence shown in SEQ ID NO: 29.

A polypeptide according to the invention having for major facilitator superfamily transporter putative furanic MFS-type transporter mfsl activity comprises the amino acid sequence set out in SEQ ID NO: 31, or an amino acid sequence encoded by the nucleotide sequence of SEQ ID NO: 32 or a variant polypeptide thereof, wherein the variant has at least 79% sequence identity or more with the sequence set out in SEQ ID NO: 32. An embodiment of the invention is a polynucleotide which comprises: (a) the nucleotide sequence set out in SEQ ID NO: 32; (b) a nucleotide sequence which hybridizes selectively with a polynucleotide being the reverse complement of SEQ ID NO: 32; (c) a nucleotide sequence having at least 79% sequence identity or more with the nucleotide sequence of SEQ ID NO: 32; (d) a fragment of a nucleotide sequence as defined in (a), (b) or (c) which is at least about 100 nucleotides in length; (e) a sequence which is degenerate as a result of the genetic code to a sequence as defined in any one of (a), (b), (c) or (d); (f) a nucleotide sequence which is the reverse complement of a nucleotide sequence as defined in (a), (b), (c), (d) or (e).

In one embodiment the variant nucleic acid molecule comprises a nucleotide sequence encoding a protein, wherein the variant nucleic acid molecule comprises a substantially homologous nucleotide sequence of at least 79%, 80%, 85%, 90%, 91%, 92%, 93%, 94%, 95%, 96%, 97%, 98%, 99% or more homologous to the nucleotide sequence shown in SEQ ID NO: 32.

In another embodiment the variant protein comprises a substantially homologous amino acid sequence of at least 32%, 33%, 35%, 40%, 45%, 50%, 55%, 60%, 65%, 70%, 75%, 80%, 85%, 90%, 91%, 92%, 93%, 94%, 95%, 96%, 97%, 98%, 99% or more homologous to the amino acid sequence shown in SEQ ID NO: 31.

A polypeptide according to the invention having LysR type transcriptional regulator activity comprises the amino acid sequence set out in SEQ ID NO: 33 or an amino acid sequence encoded by the nucleotide sequence of SEQ ID NO: 34 or a variant polypeptide thereof, wherein the variant has at least 46% sequence identity or more with the sequence set out in SEQ ID NO: 33.

An embodiment of the invention is a polynucleotide which comprises: (a) the nucleotide sequence set out in SEQ ID NO: 34;(b) a nucleotide sequence which hybridizes selectively with a polynucleotide being the reverse complement of SEQ ID NO: 34;(c) a nucleotide sequence having at least 65% sequence identity or more with the nucleotide sequence of SEQ ID NO: 34; (d) a fragment of a nucleotide sequence as defined in (a), (b) or (c) which is at least about 100 nucleotides in length; (e) a sequence which is degenerate as a result of the genetic code to a sequence as defined in any one of (a), (b), (c) or (d); (f) a nucleotide sequence which is the reverse complement of a nucleotide sequence as defined in (a), (b), (c), (d) or (e).

In one embodiment the variant nucleic acid molecule comprises a nucleotide sequence encoding a protein, wherein the variant nucleic acid molecule comprises a substantially homologous nucleotide sequence of at least 65%, 70%, 75%, 80%, 85%, 90%, 91%, 92%, 93%, 94%, 95%, 96%, 97%, 98%, 99% or more homologous to the nucleotide sequence shown in SEQ ID NO: 34.

In another embodiment the variant protein comprises a substantially homologous amino acid sequence of at least 46%, 50%, 55%, 60%, 65%, 70%, 76%, 80%, 85%, 90%, 91%, 92%, 93%, 94%, 95%, 96%, 97%, 98%, 99% or more homologous to the amino acid sequence shown in SEQ ID NO: 33.

A polypeptide according to the invention having Furoyl-CoA dehydrogenase (large subunit) activity comprises the amino acid sequence set out in SEQ ID NO: 35 or an amino acid sequence encoded by the nucleotide sequence of SEQ ID NO: 36 or a variant polypeptide thereof, wherein the variant has at least 54% sequence identity or more with the sequence set out in SEQ ID NO: 35.

An embodiment of the invention is a polynucleotide which comprises: (a) the nucleotide sequence set out in SEQ ID NO: 36; (b) a nucleotide sequence which hybridizes selectively with a polynucleotide being the reverse complement of SEQ ID NO: 36; (c) a nucleotide sequence having at least 66% sequence identity or more with the nucleotide sequence of SEQ ID NO: 36; (d) a fragment of a nucleotide sequence as defined in (a), (b) or (c) which is at least about 100 nucleotides in length; (e) a sequence which is degenerate as a result of the genetic code to a sequence as defined in any one of (a), (b), (c) or (d); (f) a nucleotide sequence which is the reverse complement of a nucleotide sequence as defined in (a), (b), (c), (d) or (e).

In one embodiment the variant nucleic acid molecule comprises a nucleotide sequence encoding a protein, wherein the variant nucleic acid molecule comprises a substantially homologous nucleotide sequence of at least 66%, 70%, 75%, 80%, 85%, 90%, 91%, 92%, 93%, 94%, 95%, 96%, 97%, 98%, 99% or more homologous to the nucleotide sequence shown in SEQ ID NO: 36.

In another embodiment the variant protein comprises a substantially homologous amino acid sequence of at least 54%, 55%, 60%, 65%, 70%, 75%, 80%, 85%, 90%, 91%, 92%, 93%, 94%, 95%, 96%, 97%, 98%, 99% or more homologous to the amino acid sequence shown in SEQ ID NO: 35.

A polypeptide according to the invention having Furoyl-CoA dehydrogenase FAD binding subunit activity which comprises the amino acid sequence set out in SEQ ID NO: 37 or an amino acid sequence encoded by the nucleotide sequence of SEQ ID NO: 38 or a variant polypeptide thereof, wherein the variant has at least 49% sequence identity or more with the sequence set out in SEQ ID NO: 37.

An embodiment of the invention is a polynucleotide which comprises: (a) the nucleotide sequence set out in SEQ ID NO: 38; (b) a nucleotide sequence which hybridizes selectively with a polynucleotide being the reverse complement of SEQ ID NO: 38; (c) a nucleotide sequence having at least 71% sequence identity or more with the nucleotide sequence of SEQ ID NO: 38; (d) a fragment of a nucleotide sequence as defined in (a), (b) or (c) which is at least about 100 nucleotides in length; (e) a sequence which is degenerate as a result of the genetic code to a sequence as defined in any one of (a), (b), (c) or (d); (f) a nucleotide sequence which is the reverse complement of a nucleotide sequence as defined in (a), (b), (c), (d) or (e).

In one embodiment the variant nucleic acid molecule comprises a nucleotide sequence encoding a protein, wherein the variant nucleic acid molecule comprises a substantially homologous nucleotide sequence of at least 71%, 75%, 80%, 85%, 90%, 91%, 92%, 93%, 94%, 95%, 96%, 97%, 98%, 99% or more homologous to the nucleotide sequence shown in SEQ ID NO: 38.

In another embodiment the variant protein comprises a substantially homologous amino acid sequence of at least 49%, 50%, 55%, 58%, 60%, 65%, 70%, 75%, 80%, 85%, 90%, 91%, 92%, 93%, 94%, 95%, 96%, 97%, 98%, 99% or more homologous to the amino acid sequence shown in SEQ ID NO: 37.

A polypeptide according to the invention having Furoyl-CoA dehydrogenase 2Fe-2S iron sulfur subunit activity which comprises the amino acid sequence set out in SEQ ID NO: 39 or an amino acid sequence encoded by the nucleotide sequence of SEQ ID NO: 40 or a variant polypeptide thereof, wherein the variant has at least 64% sequence identity or more with the sequence set out in SEQ ID NO: 39.

An embodiment of the invention is a polynucleotide which comprises: (a) the nucleotide sequence set out in SEQ ID NO: 40; (b) a nucleotide sequence which hybridizes selectively with a polynucleotide being the reverse complement of SEQ ID NO: 40; (c) a nucleotide sequence having at least 70% sequence identity or more with the nucleotide sequence of SEQ ID NO: 40; (d) a fragment of a nucleotide sequence as defined in (a), (b) or (c) which is at least about 100 nucleotides in length; (e) a sequence which is degenerate as a result of the genetic code to a sequence as defined in any one of (a), (b), (c) or (d); (f) a nucleotide sequence which is the reverse complement of a nucleotide sequence as defined in (a), (b), (c), (d) or (e).

In one embodiment the variant nucleic acid molecule comprises a nucleotide sequence encoding a protein, wherein the variant nucleic acid molecule comprises a substantially homologous nucleotide sequence of at least 70%, 75%, 80%, 85%, 90%, 91%, 92%, 93%, 94%, 95%, 96%, 97%, 98%, 99% or more homologous to the nucleotide sequence shown in SEQ ID NO: 40.

In another embodiment the variant protein comprises a substantially homologous amino acid sequence of at least 64%, 65%, 70%, 75%, 80%, 85%, 90%, 91%, 92%, 93%, 94%, 95%, 96%, 97%, 98%, 99% or more homologous to the amino acid sequence shown in SEQ ID NO: 39.

A polypeptide according to the invention having Furoyl-CoA synthetase activity which comprises the amino acid sequence set out in SEQ ID NO: 41 or an amino acid sequence encoded by the nucleotide sequence of SEQ ID NO: 42 or a variant polypeptide thereof, wherein the variant has at least 57% sequence identity or more with the sequence set out in SEQ ID NO: 41.

An embodiment of the invention is a polynucleotide which comprises: (a) the nucleotide sequence set out in SEQ ID NO: 42; (b) a nucleotide sequence which hybridizes selectively with a polynucleotide being the reverse complement of SEQ ID NO: 42; (c) a nucleotide sequence having at least 68% sequence identity or more with the nucleotide sequence of SEQ ID NO: 42; (d) a fragment of a nucleotide sequence as defined in (a), (b) or (c) which is at least about 100 nucleotides in length; (e) a sequence which is degenerate as a result of the genetic code to a sequence as defined in any one of (a), (b), (c) or (d); (f) a nucleotide sequence which is the reverse complement of a nucleotide sequence as defined in (a), (b), (c), (d) or (e).

In one embodiment the variant nucleic acid molecule comprises a nucleotide sequence encoding a protein, wherein the variant nucleic acid molecule comprises a substantially homologous nucleotide sequence of at least 68%, 70%, 75%, 80%, 85%, 90%, 91%, 92%, 93%, 94%, 95%, 96%, 97%, 98%, 99% or more homologous to the nucleotide sequence shown in SEQ ID NO: 42.

In another embodiment the variant protein comprises a substantially homologous amino acid sequence of at least 57%, 60%, 65%, 70%, 75%, 80%, 85%, 90%, 91%, 92%, 93%, 94%, 95%, 96%, 97%, 98%, 99% or more homologous to the amino acid sequence shown in SEQ ID NO: 41.

A polypeptide according to the invention having 2-oxoglutaroyl-CoA hydrolase activity comprises the amino acid sequence set out in SEQ ID NO: 43 or an amino acid sequence encoded by the nucleotide sequence of SEQ ID NO: 44 or a variant polypeptide thereof, wherein the variant has at least 72% sequence identity or more with the sequence set out in SEQ ID NO: 44.

An embodiment of the invention is a polynucleotide which comprises: (a) the nucleotide sequence set out in SEQ ID NO: 44; (b) a nucleotide sequence which hybridizes selectively with a polynucleotide being the reverse complement of SEQ ID NO: 44; (c) a nucleotide sequence having at least 74% sequence identity or more with the nucleotide sequence of SEQ ID NO: 44; (d) a fragment of a nucleotide sequence as defined in (a), (b) or (c) which is at least about 100 nucleotides in length; (e) a sequence which is degenerate as a result of the genetic code to a sequence as defined in any one of (a), (b), (c) or (d); (f) a nucleotide sequence which is the reverse complement of a nucleotide sequence as defined in (a), (b), (c), (d) or (e).

In one embodiment the variant nucleic acid molecule comprises a nucleotide sequence encoding a protein, wherein the variant nucleic acid molecule comprises a substantially homologous nucleotide sequence of at least 74%, 75%, 80%, 85%, 90%, 91%, 92%, 93%, 94%, 95%, 96%, 97%, 98%, 99% or more homologous to the nucleotide sequence shown in SEQ ID NO: 44.

In another embodiment the variant protein comprises a substantially homologous amino acid sequence of at least 72%, 75%, 80%, 85%, 90%, 91%, 92%, 93%, 94%, 95%, 96%, 97%, 98%, 99% or more homologous to the amino acid sequence shown in SEQ ID NO: 43.

A polypeptide according to the invention having putative furanic MFS-type transporter (major facilitator superfamily transporter mfs2) activity comprises the amino acid sequence set out in SEQ ID NO: 45, or an amino acid sequence encoded by the nucleotide sequence of SEQ ID NO: 46 or a variant polypeptide thereof, wherein the variant has at least 29% sequence identity or more with the sequence set out in SEQ ID NO: 46. An embodiment of the invention is a polynucleotide which comprises: (a) the nucleotide sequence set out in SEQ ID NO: 46; (b) a nucleotide sequence which hybridizes selectively with a polynucleotide being the reverse complement of SEQ ID NO: 46; (c) a nucleotide sequence having at least 79% sequence identity or more with the nucleotide sequence of SEQ ID NO: 46; (d) a fragment of a nucleotide sequence as defined in (a), (b) or (c) which is at least about 100 nucleotides in length; (e) a sequence which is degenerate as a result of the genetic code to a sequence as defined in any one of (a), (b), (c) or (d); (f) a nucleotide sequence which is the reverse complement of a nucleotide sequence as defined in (a), (b), (c), (d) or (e).

In one embodiment the variant nucleic acid molecule comprises a nucleotide sequence encoding a protein, wherein the variant nucleic acid molecule comprises a substantially homologous nucleotide sequence of at least 79%, 80%, 85%, 90%, 91%, 92%, 93%, 94%, 95%, 96%, 97%, 98%, 99% or more homologous to the nucleotide sequence shown in SEQ ID NO: 46.

In another embodiment the variant protein comprises a substantially homologous amino acid sequence of at least 29%, 30%, 35%, 40%, 45%, 50% 55%, 60%, 65%, 70%, 75%, 80%, 85%, 90%, 91%, 92%, 93%, 94%, 95%, 96%, 97%, 98%, 99% or more homologous to the nucleotide sequence shown in SEQ ID NO: 45.

A further embodiment is a vector incorporating the polynucleotide sequences or a nucleic acid constructs set out above.

The terms “homology”, “sequence identity” and the like are used interchangeably herein. For the purpose of this invention, it is defined herein that in order to determine the degree of sequence identity shared by two amino acid sequences or by two nucleic acid sequences, the sequences are aligned for optimal comparison purposes (e.g., gaps can be introduced in the sequence of a first amino acid or nucleic acid sequence for optimal alignment with a second amino or nucleic acid sequence). Such alignment may be carried out over the full lengths of the sequences being compared. Alternatively, the alignment may be carried out over a shorter comparison length, for example over about 20, about 50, about 100 or more nucleic acids/bases or amino acids. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The degree of identity shared between sequences is typically expressed in term of percentage identity between the two sequences and is a function of the number of identical positions shared by the sequences (i.e., % identity=number of identical positions/total number of positions (i.e. overlapping positions)×100). Preferably, the two sequences being compared are of the same or substantially the same length.

The skilled person will be aware of the fact that several different computer programs are available to determine the homology between two sequences. For instance, a comparison of sequences and determination of percent identity between two sequences can be accomplished using a mathematical algorithm. In a preferred embodiment, the percentage identity between two amino acid sequences is determined using the Needleman and Wunsch (J. Mol. Biol. (48): 444-453 (1970)) algorithm which has been incorporated into the GAP program in the Accelrys GCG software package (available at http://www.accelrys.com/products/gcg/), using either a Blossom 62 matrix or a PAM250 matrix, and a gap weight of 16, 14, 12, 10, 8, 6, or 4 and a length weight of 1, 2, 3, 4, 5, or 6. The skilled person will appreciate that all these different parameters will yield slightly different results but that the overall percentage identity of two sequences is not significantly altered when using different algorithms.

In yet another embodiment, the percent identity between two nucleotide sequences is determined using the GAP program in the Accelrys GCG software package (available at http://www.accelrys.com/products/gcg/), using a NWSgapdna.CMP matrix and a gap weight of 40, 50, 60, 70, or 80 and a length weight of 1, 2, 3, 4, 5, or 6. In another embodiment, the percent identity two amino acid or nucleotide sequence is determined using the algorithm of E. Meyers and W. Miller (CABIOS, 4:11-17 (1989) which has been incorporated into the ALIGN program (version 2.0) (available at the ALIGN Query using sequence data of the Genestream server IGH Montpellier France http://vega.igh.cnrs.fr/bin/align-guess.cgi) using a PAM120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences for instance homologous to oxidoreductase encoding nucleic acid molecules of the invention. BLAST protein searches can be performed with the XBLAST program, score=50, wordlength=3 to obtain amino acid sequences homologous to oxidoreductase protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., XBLAST and NBLAST, also known as BLASTn and BLASTx) can be used. See the homepage of the National Center for Biotechnology Information at http://www.ncbi.nlm.nih.gov/.

As used herein, the term “selectively hybridizing”, “hybridizes selectively” and similar terms are intended to describe conditions for hybridization and washing under which nucleotide sequences at least at least 70%, at least 75%, at least 80%, more preferably at least 85%, even more preferably at least 90%, preferably at least 95%, more preferably at least 98% or more preferably at least 99% homologous to each other typically remain hybridized to each other. That is to say, such hybridizing sequences may share at least at least 70%, at least 75%, at least 80%, more preferably at least 85%, even more preferably at least 90%, more preferably at least 95%, more preferably at least 98% or more preferably at least 99% sequence identity.

A preferred, non-limiting example of such hybridization conditions is hybridization in 6× sodium chloride/sodium citrate (SSC) at about 45° C., followed by one or more washes in 1×SSC, 0.1% SDS at about 50° C., preferably at about 55° C., preferably at about 60° C. and even more preferably at about 65° C.

Highly stringent conditions include, for example, hybridization at about 68° C. in 5×SSC/5× Denhardt's solution/1.0% SDS and washing in 0.2×SSC/0.1% SDS at room temperature. Alternatively, washing may be performed at 42° C.

The skilled artisan will know which conditions to apply for stringent and highly stringent hybridization conditions. Additional guidance regarding such conditions is readily available in the art, for example, in Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.).

Of course, a polynucleotide which hybridizes only to a poly A sequence (such as the 3′ terminal poly(A) tract of mRNAs), or to a complementary stretch of T (or U) resides, would not be included in a polynucleotide of the invention used to specifically hybridize to a portion of a nucleic acid of the invention, since such a polynucleotide would hybridize to any nucleic acid molecule containing a poly (A) stretch or the complement thereof (e.g., practically any double-standed cDNA clone).

In a typical approach, gene libraries constructed from other organisms, e.g. a bacterium, in particular from the micro-organism family Trichomaceae, for example from the genus Burkholderia can be screened such as Burkholderia phytofirmans.

For example, Burkholderia strains can be screened for homologous encoding polynucleotides according to the invention by Southern blot analysis. Upon detection of homologous DNA restriction fragments according to the invention, gene libraries can be constructed from chromosomal fragments of the same size from the appropriate strain, utilizing standard techniques well known to those of skill in the art. Alternatively, if the microorganism is a eukaryote, the mRNA transcript of the respective genes according to the invention can be identified by Northen hybridization and upon identification of the transcript, cDNA libraries can be prepared using total RNA isolated from the eukaryotic microorganism. Homologous gene sequences can be isolated, for example, by performing PCR using two degenerate oligonucleotide primer pools designed on the basis of nucleotide sequences as taught herein. The template for the reaction can be total chromosomal DNA from the strain know or suspected to express a polynucleotide according to the invertion. The PCR product can be subcloned and sequenced to ensure that the amplified sequences represent the sequences of a new oxidoreductase nucleic acid sequence, or a functional equivalent thereof.

Alternatively the template for the reaction can be cDNA obtained by reverse transcription of mRNA prepared from strains known or suspected to express a polynucleotide according to the invention. The PCR product can be subcloned and sequenced to ensure that the amplified sequences represent the sequences of a new oxidoreductase nucleic acid sequence, or a functional equivalent thereof.

The PCR fragment can then be used to isolate a full-length cDNA clone by a variety of known methods. For example, the amplified fragment can be labeled and used to screen a bacteriophage or cosmid cDNA library. Alternatively, the labeled fragment can be used to screen a genomic library.

PCR technology also can be used to isolate full-length cDNA sequences from other organisms. For example, RNA can be isolated, following standard procedures, from an appropriate cellular or tissue source. A reverse transcription reaction can be performed on the RNA using an oligonucleotide primer specific for the most 5′ end of the amplified fragment for the priming of first strand synthesis.

The resulting RNA/DNA hybrid can then be “tailed” (e.g., with guanines) using a standard terminal transferase reaction, the hybrid can be digested with RNase H, and second strand synthesis can then be primed (e.g., with a poly-C primer). Thus, cDNA sequences upstream of the amplified fragment can easily be isolated. For a review of useful cloning strategies, see e.g., Sambrook et al., supra; and Ausubel et al., supra.

Another aspect of the invention pertains to vectors, including cloning and expression vectors, comprising a polynucleotide of the invention encoding a oxidoreductase protein or a functional equivalent thereof and methods of growing, transforming or transfecting such vectors in a suitable host cell, for example under conditions in which expression of a polypeptide of the invention occurs. As used herein, the term “vector” refers to a nucleic acid molecule capable of transporting another nucleic acid to which it has been linked.

Polynucleotides of the invention can be incorporated into a recombinant replicable vector, for example a cloning or expression vector. The vector may be used to replicate the nucleic acid in a compatible host cell. Thus in a further embodiment, the invention provides a method of making polynucleotides of the invention by introducing a polynucleotide of the invention into a replicable vector, introducing the vector into a compatible host cell, and growing the host cell under conditions which bring about replication of the vector. The vector may be recovered from the host cell. Suitable host cells are described below.

The vector into which the expression cassette or polynucleotide of the invention is inserted may be any vector which may conveniently be subjected to recombinant DNA procedures, and the choice of the vector will often depend on the host cell into which it is to be introduced.

A vector according to the invention may be an autonomously replicating vector, i. e. a vector which exists as an extra-chromosomal entity, the replication of which is independent of chromosomal replication, e.g. a plasmid. Alternatively, the vector may be one which, when introduced into a host cell, is integrated into the host cell genome and replicated together with the chromosome (s) into which it has been integrated.

One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. The terms “plasmid” and “vector” can be used interchangeably herein as the plasmid is the most commonly used form of vector. However, the invention is intended to include such other forms of expression vectors, such as cosmid, viral vectors (e.g., replication defective retroviruses, adenoviruses and adeno-associated viruses) and phage vectors which serve equivalent functions.

Vectors according to the invention may be used in vitro, for example for the production of RNA or used to transfect or transform a host cell.

A vector of the invention may comprise two or more, for example three, four or five, polynucleotides of the invention, for example for overexpression.

The recombinant expression vectors of the invention comprise a nucleic acid of the invention in a form suitable for expression of the nucleic acid in a host cell, which means that the recombinant expression vector includes one or more regulatory sequences, selected on the basis of the host cells to be used for expression, which is operably linked to the nucleic acid sequence to be expressed.

Within a vector, such as an expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell), i.e. the term “operably linked” refers to a juxtaposition wherein the components described are in a relationship permitting them to function in their intended manner. A regulatory sequence such as a promoter, enhancer or other expression regulation signal “operably linked” to a coding sequence is positioned in such a way that expression of the coding sequence is achieved under condition compatible with the control sequences or the sequences are arranged so that they function in concert for their intended purpose, for example transcription initiates at a promoter and proceeds through the DNA sequence encoding the polypeptide.

The term “regulatory sequence” or “control sequence” is intended to include promoters, enhancers and other expression control elements (e.g., polyadenylation signal). Such regulatory sequences are described, for example, in Goeddel; Gene Expression Technology: Methods in Enzymology 185, Academic Press, San Diego, Calif. (1990).

The term regulatory or control sequences includes those sequences which direct constitutive expression of a nucleotide sequence in many types of host cells and those which direct expression of the nucleotide sequence only in a certain host cell (e.g. tissue-specific regulatory sequences).

A vector or expression construct for a given host cell may thus comprise the following elements operably linked to each other in a consecutive order from the 5′-end to 3′-end relative to the coding strand of the sequence encoding the polypeptide of the first invention: (1) a promoter sequence capable of directing transcription of the nucleotide sequence encoding the polypeptide in the given host cell; (2) optionally, a signal sequence capable of directing secretion of the polypeptide from the given host cell into a culture medium; (3) a DNA sequence of the invention encoding a mature and preferably active form of a polypeptide having cellobiohydrolase activity; and preferably also (4) a transcription termination region (terminator) capable of terminating transcription downstream of the nucleotide sequence encoding the polypeptide.

Downstream of the nucleotide sequence according to the invention there may be a 3′ untranslated region containing one or more transcription termination sites (e.g. a terminator). The origin of the terminator is less critical. The terminator can, for example, be native to the DNA sequence encoding the polypeptide. However, preferably a yeast terminator is used in yeast host cells and a filamentous fungal terminator is used in filamentous fungal host cells. More preferably, the terminator is endogenous to the host cell (in which the nucleotide sequence encoding the polypeptide is to be expressed). In the transcribed region, a ribosome binding site for translation may be present. The coding portion of the mature transcripts expressed by the constructs will include a translation initiating AUG at the beginning and a termination codon appropriately positioned at the end of the polypeptide to be translated.

Enhanced expression of the polynucleotide of the invention may also be achieved by the selection of heterologous regulatory regions, e. g. promoter, secretion leader and/or terminator regions, which may serve to increase expression and, if desired, secretion levels of the protein of interest from the expression host and/or to provide for the inducible control of the expression of a polypeptide of the invention.

It will be appreciated by those skilled in the art that the design of the expression vector can depend on such factors as the choice of the host cell to be transformed, the level of expression of protein desired, etc. The vectors, such as expression vectors, of the invention can be introduced into host cells to thereby produce proteins or peptides, encoded by nucleic acids as described herein (e.g. in the case of hmfH, oxidoreductase proteins, mutant forms of oxidoreductase proteins, fragments, variants or functional equivalents thereof, fusion proteins, etc.). The same applies to the other polypeptides according to the invention.

The vectors, such as recombinant expression vectors, of the invention can be designed for expression of suitable proteins in prokaryotic or eukaryotic cells. For example oxidoreductase proteins can be expressed in bacterial cells such as E. coli, insect cells (using baculovirus expression vectors), filamentous fungi, yeast cells or mammalian cells. Suitable host cells are discussed further in Goeddel, Gene Expression Technology: Methods in Enzymology 185, Academic Press, San Diego, Calif. (1990). Representative examples of appropriate hosts are described hereafter. Appropriate culture mediums and conditions for the above-described host cells are known in the art.

As set out above, the term “control sequences” or “regulatory sequences” is defined herein to include at least any component which may be necessary and/or advantageous for the expression of a polypeptide. Any control sequence may be native or foreign to the nucleic acid sequence of the invention encoding a polypeptide. Such control sequences may include, but are not limited to, a promoter, a leader, optimal translation initiation sequences (as described in Kozak, 1991, J. Biol. Chem. 266:19867-19870), a secretion signal sequence, a pro-peptide sequence, a polyadenylation sequence, a transcription terminator. At a minimum, the control sequences typically include a promoter, and transcriptional and translational stop signals.

A stably transformed microorganism is one that has had one or more DNA fragments introduced such that the introduced molecules are maintained, replicated and segregated in a growing culture. Stable transformation may be due to multiple or single chromosomal integration (s) or by (an) extrachromosomal element(s) such as (a) plasmid vector(s). A plasmid vector is capable of directing the expression of polypeptides encoded by particular DNA fragments. Expression may be constitutive or regulated by inducible (or repressible) promoters that enable high levels of transcription of functionally associated DNA fragments encoding specific polypeptides.

Isolation of the polypeptides according to the invention: one or more polypeptides according to the invention or DNA material expressing the polypeptides according to the invention may be isolated from an organism, preferably a microorganism that expresses the oxidoreductase.

Preferably, the microorganism is capable of using furanic compounds, preferably HMF and/or furufal as a substrate, more preferably not using other carbon sources such as C5 and/or C6 sugars. The microorganism preferably is chosen from the group consisting of: Cupriavidus (preferably Cupriavidus basilensis, Cupriavidus Eutropha and/or Cupriavidus basilensis HMF14,), Burkholderia (prefereably Burkholderia phytofirmans and/or Burkholderia phytofirmans PsJN), Bradyhrizobium (preferably, Bradyhrizobium japonicum, and/or Bradyhrizobium japonicum USDA110), Methylobacterium (preferably Methylobacterium radiotolerans and/or Methylobacterium radiotolerans JCM2831).

Most preferred polypeptides useful in the present invention are converting furfural and/or HMF as substrate, and are polypeptides according to the invention isolated from Cupriavidus basilensis HMF 14 herein, deposited in accordance with the Budapest Treaty on International Recognition of the Deposits of Microorganisms for the Purpose of Patent Procedures at the Deutsche Sammlung von Mikroorganismen and Zellkulturen GmbH (German Collection of Microorganisms and Cell Cultures), InhoffenstraSe 7B, 38124 Braunschweig, GERMANY as strain HMF 14, having deposit number DSM 22875; deposition date of Aug. 19, 2009.

The present invention thus also relates to the isolated HMF-utilizing bacterium, Cupriavidus basilensis strain HMF14, and its genes involved in the HMF degradative pathway.

One of the genes (herein defined as hmfH) encodes a 579-amino acid, 62 kDa FAD-dependent oxidoreductase that was found to oxidize furfuryl alcohol, furfural, HMF, and 5-hydroxymethyl-furoic acid.

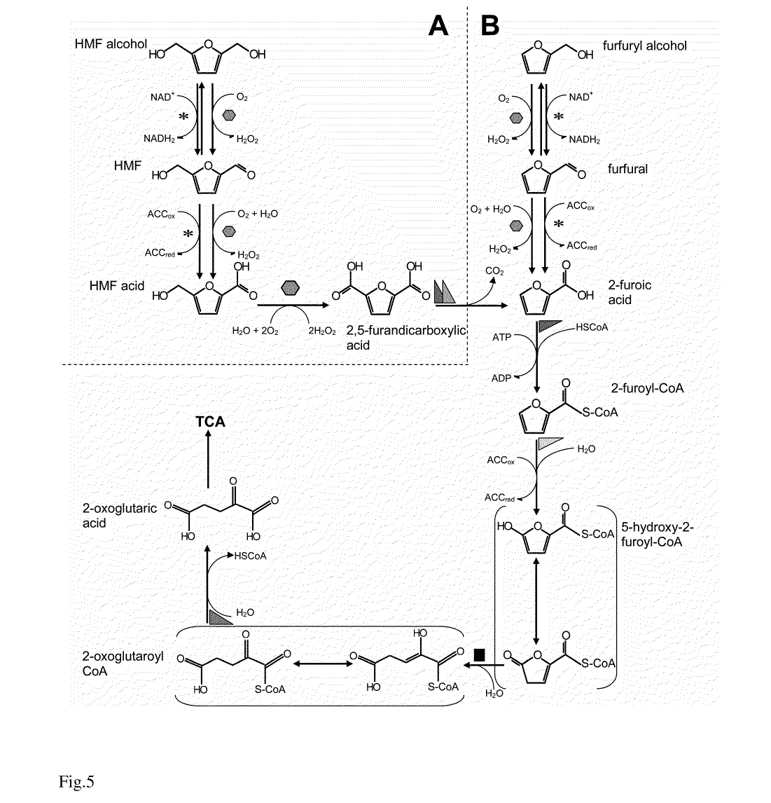

The alcohol/aldehyde groups at C2 and C5 in these molecules are oxidized, to yield i.e. furan-dicarboxylic acid from HMF, respectively, and 5-furoic acid from furfurylalcohol or furfural (see FIG. 5).

The present invention thus provides polynucleotides encoding polypeptides, having the following activity:

Furoyl-CoA dehydrogenase large subunit; a Furoyl-CoA dehydrogenase FAD binding subunit; Furoyl-CoA dehydrogenase 2Fe-2S iron sulfur subunit; a Furoyl-CoA synthetase; a 2-oxoglutaroyl-CoA hydrolase; a 2,5-furan-dicarboxylic acid decarboxylase 1, a 2,5-furan-dicarboxylic acid decarboxylase 2; a HMF/furfural oxidoreductase; a LysR type transcriptional regulator 1 and a LysR type transcriptional regulator 2, an aldehyde dehyrogenase and major family supertransporters 1 and 2.

Enzymes are herein understood as a subclass of polypeptides. A preferred furanic-compound-removing pathway useful in the present invention uses a novel Furoyl-CoA dehydrogenase, a novel Furoyl-CoA synthetase, a novel 2-oxoglutaroyl-CoA hydrolase, two novel 2,5-furan-dicarboxylic acid decarboxylases (1 and 2), and/or a novel HMF/furfural oxidoreductase isolated from Cupriavidus basilensis HMF 14 herein, deposited in accordance with the Budapest Treaty on International Recognition of the Deposits of Microorganisms for the Purpose of Patent Procedures as Deposit number DSM 22875.

Lignocellulose hydrolysate is the result of subjecting lignocellulosic material to a pre-treatment step. Suitable lignocellulose-containing material according to the subject invention includes, but is not limited to wood such as wood chips, saw dust; municipal waste containing lignocellulose; waste paper pulp; perennial grasses such as switchgrass (panicum virgatum); miscanthus species such as miscanthus x giganteus, miscanthus sinensis and miscanthus sacchariflorus; energy cane, sugar cane; sweet sorghum; corn cobs and corn stovers, wheat straw, rice straw and other sources of lignocellulosic material.

Lignocellulose is one of the most abundant plant material resources in the world. However, an effective pretreatment is needed to remove the rigid crystalline structure, enforced by lignin and hemicellulose to render it accessible for a subsequent hydrolysis step.

The pretreatment preferably includes both physical and chemical pretreatment steps. Physical pretreatment is often called size reduction to reduce biomass physical size. Chemical pretreatment is to remove chemical barriers so that the enzymes can access cellulose degradation.

Currently used pretreatment techniques include acid hydrolysis, steam explosion, ammonia fiber expansion, organosolve and sulfite pretreatment, alkaline wet oxidation and ozone pretreatment.

Most pretreatment techniques result in the formation of degradation products that have inhibitory effects on subsequent hydrolysis and fermentation processes. The presence of inhibitors will not only further complicate the ethanol production but also increase the cost of production due to entailed detoxification steps.

The method most often applied includes acid hydrolysis, where the lignocellulosic material is subjected to an acid such as sulphuric acid, whereby the sugar polymers cellulose and hemicellulose are partly or completely hydrolysed to their constituent sugar monomers. Another type of lignocellulose hydrolysis is steam explosion, a process comprising heating of the lignocellulosic material by steam injection to a temperature of 190-230° C. A third method is wet oxidation wherein the material is treated with oxygen at 150-185° C. All methods may also be combined, e.g. steam explosion or oxidation in presence of acids. These pretreatments may be followed by enzymatic hydrolysis to complete the release of sugar monomers. The pretreatment steps results in the hydrolysis of cellulose into glucose while hemicellulose is transformed into the pentoses xylose and arabinose and the hexoses glucose, galactose and mannose.

The pretreatment step may in certain embodiments be supplemented with treatment resulting in further hydrolysis of the cellulose and hemicellulose. The purpose of such an additional hydrolysis treatment is to hydrolyse oligosaccharide and possibly polysaccharide species produced during the acid hydrolysis, wet oxidation, or steam explosion of cellulose and/or hemicellulose origin to form fermentable sugars (e.g. glucose, xylose and possibly other monosaccharides). Such further treatments may be either chemical or enzymatic. Chemical hydrolysis is typically achieved by treatment with an acid, such as treatment with aqueous sulphuric acid or formic acid, at a temperature in the range of about 100-150° C. Enzymatic hydrolysis is typically performed by treatment with one or more appropriate carbohydrase enzymes such as cellulases, glucosidases and hemicellulases including xylanases.

### DETAILED DESCRIPTION OF THE INVENTION

Without wishing to be bound to any particular theory, it is believed that the inhibitory effect of lignocellulosic hydrolysate on fermentation comes from a concerted effect of many toxic constituents, but that furanic derivatives have a key role in this effect. It has been surprisingly found that C. basilensis HMF14 according to the invention is able to selectively degrade furanic derivatives in lignocellulosic hydrolysate, while essentially not utilizing fermentable sugars.

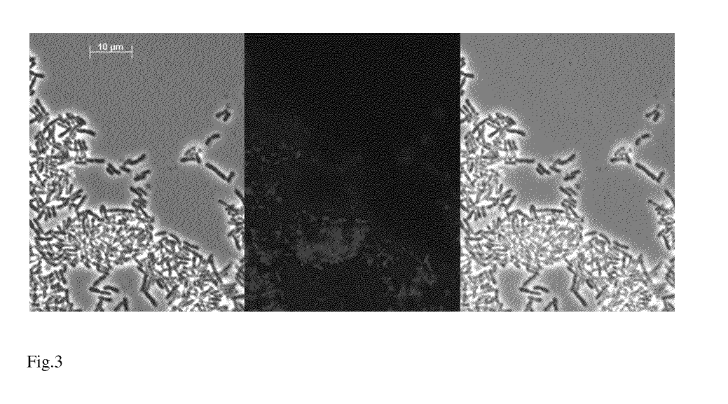

C. basilensis HMF14 was isolated on HMF, however it was found to also utilize furfural as sole carbon source. As a further beneficial effect, C. basilensis HMF14 is also able to remove the majority of non-furanic inhibitory compounds, such as acetate and formate, as illustrated in FIG. 3, which shows that the furanic derivatives, acetate and formate were completely removed from wheat straw hydrolysate after only ten hours of cultivation. During this period, the glucose, xylose and arabinose concentrations, i.e. fermentable sugars in lignocellulose hydrolysate, were stable. Only when the incubation was prolonged after these inhibitor compounds were consumed, the sugar concentration decreased by approximately 11% within 15 h. Without wishing to be bound to any particular theory, it is believed that this may indicate that the sugars are not converted, but absorbed in the mucous layer around the bacterial cells.

Accordingly, the ability to digest or convert furanic compounds without use of fermentable sugars is defined as the reduction of the inhibitor concentration while maintaining a stable fermentable sugar concentration before all furanic compounds are converted. The term “all furanic compounds are converted” refers to a concentration of furanic compounds of less than 3000 ppmw, more preferably less than 2000 ppmw, more preferably less than 1500 ppmw, more preferably less than 1000 ppmw, more preferably less than 500 ppmw, and yet more preferably less than 150 ppmw. In a further embodiment the term “all furanic compounds are converted” refers to a concentration of furanic compounds that is equal to or less than 50 wt %, more preferably equal to or less than 30 wt %, more preferably equal to or less than 10 wt %, more preferably equal to or less than 5 wt %, yet more preferably equal to or less than 1 wt %, and still more preferably equal to or less than 0.1 wt % of the concentration of furanic compounds present in the lignocellulose hydrolysate before digestion or conversion of the furanic compounds as described herein. Undiluted wheat straw hydrolysate was detoxified to completion as well, although an extended lag phase occurred that could be ameliorated by increasing the inoculum density.

As set out above, C. basilensis HMF14 was demonstrated to metabolize individual inhibitors in minimal medium. In addition to individual compounds, complex mixtures of toxic inhibitors were also efficiently metabolized as demonstrated by the detoxification of actual wheat straw hydrolysate. Treatment of lignocellulosic hydrolysate with C. basilensis HMF14 resulted in a solution of glucose, xylose and arabinose that is essentially free from furan aldehydes, acetate and formate. The unique substrate profile of C. basilensis HMF14 according to the invention makes this bacterium ideally suited for biological detoxification of lignocellulosic hydrolysate. Accordingly, the present invention further provides for an isolated culture of a Cupriavidus microorganism of Cupriavidus basilensis strain Deposit number DSM 22875 which, when provided with furanic compounds derivatives, preferably HMF and Furfural-derived compounds as a sole carbon source, grows on said source and expresses several novel enzymes, which act synergetically, comprising a novel 2-furoyl-CoA:acceptor 5-oxidoreductase (hydroxylating) EC 1.3.99.8), further referred to as Furoyl-CoA dehydrogenase, a novel Furoyl-CoA synthetase, a novel 2-oxoglutaroyl-CoA hydrolase, two novel 2,5-furan-dicarboxylic acid decarboxylases, and a novel HMF/furfural oxidoreductase.

An embodiment of the present invention is a vector incorporating the polynucleotide sequences or nucleic acid constructs set out herein-above, wherein the nucleotide sequences encode one or more of Furoyl-CoA dehydrogenase, Furoyl-CoA synthetase, 2-oxoglutaroyl-CoA hydrolase, 2,5-furan-dicarboxylic acid decarboxylase 1,2,5-furan-dicarboxylic acid decarboxylase 2 and a

HMF/furfural oxidoreductase from Cupriavidus basilensis HMF14 DSM 22875 in a host cell under conditions conducive for their expression.

The invention also relates to a host microorganism transformed or transfected by the isolated DNA or by a vector or plasmid comprising the isolated DNA according to the invention under conditions conducive to express one or more of a Furoyl-CoA dehydrogenase, a Furoyl-CoA synthetase, a 2-oxoglutaroyl-CoA hydrolase, a 2,5-furan-dicarboxylic acid decarboxylase 1, a 2,5-furan-dicarboxylic acid decarboxylase 2 and a HMF/furfural oxidoreductase.

The present invention also provides for a cell extract from a Cupriavidus microorganism or a host cell comprising one or more of a Furoyl-CoA dehydrogenase, a Furoyl-CoA synthetase, a 2-oxoglutaroyl-CoA hydrolase, a 2,5-furan-dicarboxylic acid decarboxylase 1, a 2,5-furan-dicarboxylic acid decarboxylase 2 and a HMF/furfural oxidoreductase.

These enzymes act synergetically when provided with the relevant cofactors. The invention also relates to a composition comprising the cell extract, which preferably is a fractionated soluble cytosolic fraction.

The present invention also relates to a process for the in-situ detoxification of lignocellulose hydrolysate comprising furanic compounds, preferably HMF, HMF alcohol and HMF carboxylic acid, and Furfurylalcohol, Furfural and/or Furoic acid with a suitable host microorganism, comprising contacting the lignocellulose hydrolysate with the host microorganism under conditions facilitating the expression of one ore more of the Furoyl-CoA dehydrogenase, a Furoyl-CoA synthetase, a 2-oxoglutaroyl-CoA hydrolase, a 2,5-furan-dicarboxylic acid decarboxylase 1, a 2,5-furan-dicarboxylic acid decarboxylase 2 and a HMF/furfural oxidoreductase, as required to for the conversion of the furanic compounds, from a microorganism from the family of Burkholderiaceae, preferably Cupriavidus, more preferably Cupriavidus basilensis, again more preferably Cupriavidus basilensis hmf 14 according to the invention to convert the furanic compounds to non-toxic components to obtain a detoxified lignocellulose hydrolysate. The term “toxic” refers to lignocellulose degradation products that are fermentation inhibitors that inhibit the growth of e.g. ethanologenic microorganisms employed in the fermentation step, thereby inhibiting a suitable performance of these organisms and reducing yields. The term “non-toxic” means that the conversion products of the toxic compounds are essentially not inhibiting the growth of microorganisms that ferment the cellulose components.

Preferably, the Cupriavidus microorganism is Cupriavidus basilensis HMF14 according to the invention. More preferably, the host microorganism comprises DNA encoding one or more of the following group: Furoyl-CoA dehydrogenase, Furoyl-CoA synthetase, 2-oxoglutaroyl-CoA hydrolase, 2,5-furan-dicarboxylic acid decarboxylase 1,2,5-furan-dicarboxylic acid decarboxylase 2 and HMF/furfural oxidoreductase from Cupriavidus microorganism of Cupriavidus basilensis HMF14 according to the invention. The process further preferably comprises subjecting the detoxified lignocellulose hydrolysate to a simultaneous or subsequent fermentation step. It further preferably comprises a step of pre-treating lignocellulose-containing material to obtain a lignocellulose hydrolysate, more preferably under acidic conditions.

A further aspect of the subject invention is a process comprising introducing isolated DNA sequence as set out herein above and/or (b) isolated DNA sequences which are at least sufficiently identical to the DNA sequences to encode,polypeptides having the activity of of one ore more of the Furoyl-CoA dehydrogenase, a Furoyl-CoA synthetase, a 2-oxoglutaroyl-CoA hydrolase, a 2,5-furan-dicarboxylic acid decarboxylase 1, a 2,5-furan-dicarboxylic acid decarboxylase 2 and a HMF/furfural oxidoreductase, as required to for the conversion of the furanic compounds in an appropriate host cell, cultivating the obtained host cell under conditions conducive to the detoxification of lignocelluloses hydrolysate, and recovering a detoxified lignocelluloses hydrolysate from the culture. The term “detoxification” thus refers to the conversion of toxic compounds to non-toxic compounds.

The present invention also provides for a process for the production of Furoyl-CoA dehydrogenase, Furoyl-CoA synthetase, 2-oxoglutaroyl-CoA hydrolase, 2,5-furan-dicarboxylic acid decarboxylase 1,2,5-furan-dicarboxylic acid decarboxylase 2 and/or a HMF/furfural oxidoreductase which are at least 45% identical to those expressed by Cupriavidus basilensis HMF14 DSM 22875or a host microorganism as set out above, comprising
(a) culturing a microorganism in a nutrient medium containing carbon and nitrogen sources and inorganic salts; and
(b) isolating the enzymes produced from the microorganism.

Preferably, in the process according to the invention, Furanic compounds such as HMF, Furfurylalcohol, Furfural and/or Furoic acid are converted to a polyhydroxyalkanoate (PHA), and/or to biofuels such as ethanol. The invention also provides for a process for the in-situ detoxification of lignocellulose hydrolysate with a suitable host microorganism, comprising cultivating the host microorganism in the presence of furfural, furfuryl alcohol, hydroxymethylfurfural and/or furoic acid under conditions facilitating the expression of the enzymes from a Cupriavidus microorganism. Preferably, the host microorganism is a Cupriavidus microorganism of Cupriavidus basilensis HMF 14 according to the invention. More preferably, the host microorganism comprises DNA encoding an enzyme complex comprising a Furoyl-CoA dehydrogenase, a Furoyl-CoA synthetase, a 2-oxoglutaroyl-CoA hydrolase, a 2,5-furan-dicarboxylic acid decarboxylase 1, a 2,5-furan-dicarboxylic acid decarboxylase 2 and a HMF/furfural oxidoreductase.

A further aspect of the subject invention preferably comprises subjecting the detoxified lignocellulose hydrolysate to a simultaneous or subsequent fermentation step.

Also described is a group of enzymes forming a synergetic enzyme pathway for the degradation of furanic compounds, further referred to as enzyme complex which may be isolated from Cupriavidus basilensis HFM14.

Furanic compounds, such as HMF, Furfural and furanoic acid are the primary substrates for this enzyme complex expressed from C.b. HMF 14 since this organism can grow on either compound as the sole substrate providing carbon and energy.

Also a process for producing these enzymes is provided. Thus, there is provided an improved method for the biocatalytic production of PHA and/or biofuels such as ethanol in a suitable host microorganism of the enzymatic pathway of Cupriavidus basilensis strain DSM 22875, under conditions facilitating the expression of the activity.

This embodiment preferably includes modifications of Cupriavidus basilensis strain DSM 22875 to block conversion of fermentable sugars to compounds along its degradation pathway to compounds.

The present invention further pertains to a process for the conversion of furanic compounds, such as furfuryl alcohol and/or furfural and/or furoic acid to 2-furoyl CoA, comprising contacting furfuryl alcohol and/or furfural and/or furoic acid with a furoyl-CoA dehydrogenase, furoyl-CoA synthetase, 2-oxoglutaroyl-CoA hydrolase, and 2,5-furan-dicarboxylic acid decarboxylase catalyst in the presence of one or more coenzyme cofactor. Without wqishing to be bound to any particular theory, it is believed that the furoyl-CoA synthetase converts the furanic compounds to form furoyl-CoA, while the other enzymes degrade the thus obtained furoyl-CoA. The (bio)catalyst preferably comprises a polypeptide as set out herein before for each of the polypeptide enzymes.

The present invention also pertains to a process for the conversion of furanic compounds, preferably one or more of 5-hydroxymethylfurfural (HMF), 2,5-dihydroxymethyl furan (HMF alcohol), 5-hydroxymethyl-2-furancarboxylic acid (HMF acid) and/or 2,5-furandicarboxylic acid to 2-furoyl CoA, comprising comprising contacting the Furanic compounds with a furoyl-CoA dehydrogenase, furoyl-CoA synthetase, HMF oxidoreductase and the decarboxylases catalyst in the presence of one or more coenzyme cofactor.

Preferably, the coenzyme cofactor is nicotinamide adenine dinucleotide (NAD+) and/or flavin adenine dinucleotide (FAD) and/or pyrroloquinoline quinolone (PQQ).

Yet further, its capability of producing PHA may furthermore contribute to cost effectiveness since the biomass generated may be employed for the production of bioplastics.

Similarly, conditions may be suitable to also facilitate the expression of any one or more of the enzymatic activities necessary to convert the furanic compounds to poylhydroxalkanoate.

The enzyme of the present invention is a multicomponent enzyme that can utilize nicotinamide adenine dinucleotide (NADH) or nicotinamide adenine phosphate dinucleotide (NADPH), requires flavin adenine dinucleotide (FAD) and its activity is stimulated by the presence of iron in a cell-free extract.

Also proposed is the cloning and sequencing of the gene encoding the preferred enzymes, Furoyl-CoA dehydrogenase, Furoyl-CoA synthetase, 2-oxoglutaroyl-CoA hydrolase, 2,5-furan-dicarboxylic acid decarboxylase 1,2,5-furan-dicarboxylic acid decarboxylase 2 and HMF/furfural oxidoreductase.

### DETAILED DESCRIPTION OF THE FIGURES

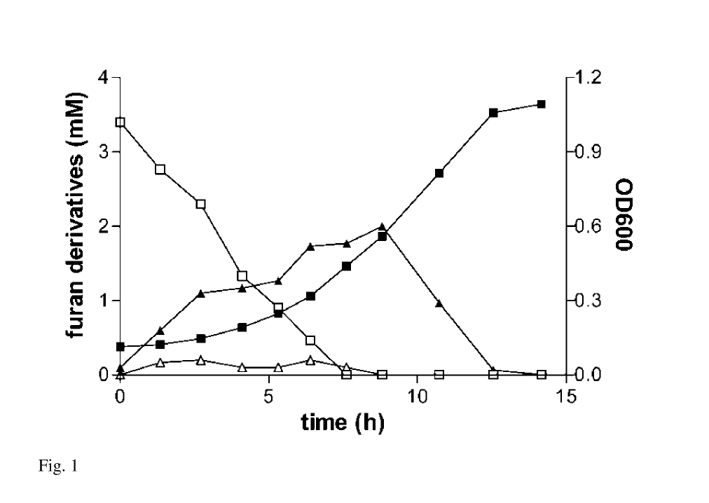

FIG. 1 shows the growth of C. basilensis HMF14 on mineral salts medium with furfural as the sole carbon source. ε, furfural; ▴, furfuryl alcohol; Δ, furoic acid; ▪, OD600. Cultures were performed in triplicate. Variations between replicates was less than 10%.

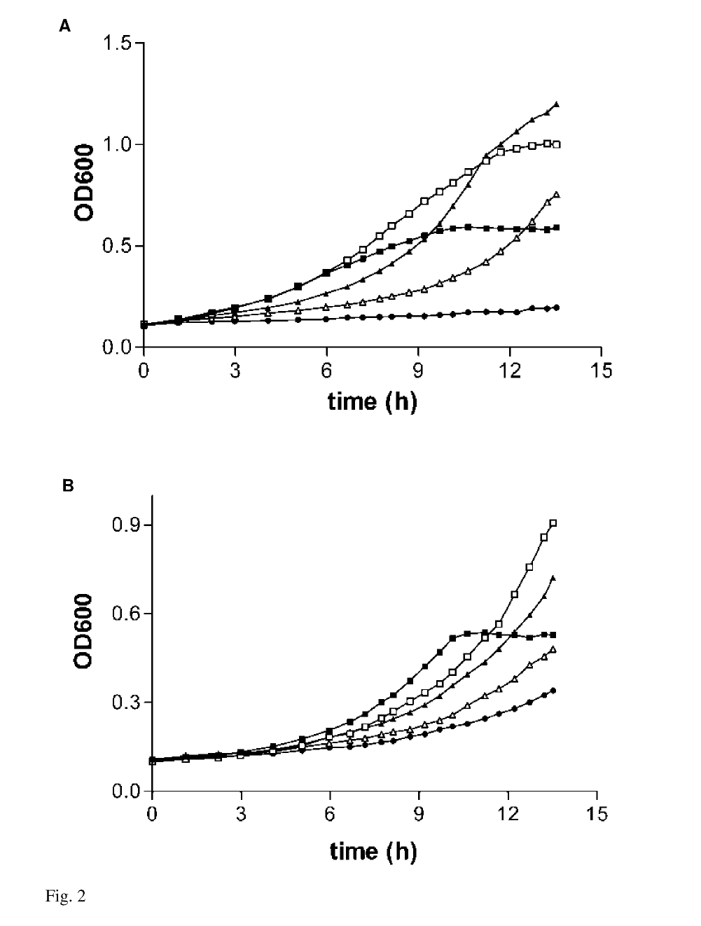

FIG. 2 illustrates the growth of C. basilensis HMF14 on different concentrations of furfural (A) or HMF (B). The concentrations used were ▪, 3 mM; □, 6 mM; ▴, 9 mM; Δ, 12 mM; , 15 mM.

FIG. 3 depicts the detection of PHA in cultures of C. basilensis HMF14 in minimal medium with 120 mM acetate. Left: phase contrast image. Middle: Fluorescence microscopic image of the same slide stained with Nile Blue A. Right: Overlay of the two previous images.

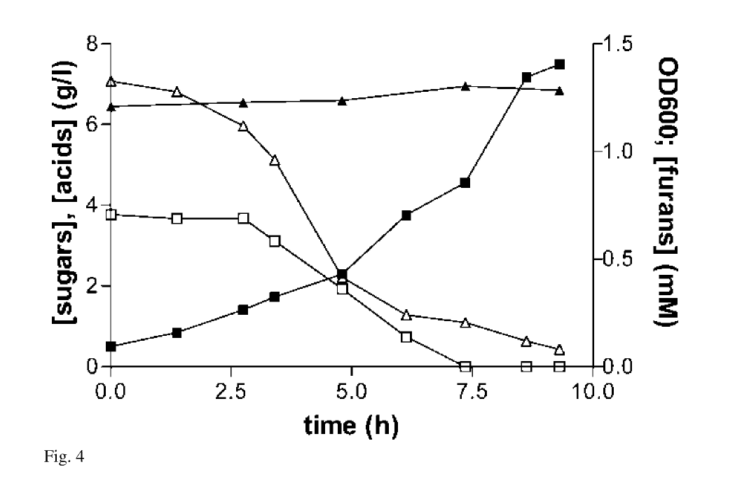

FIG. 4 illustrates the detoxification of lignocellulosic hydrolysate by C. basilensis HMF14. The sugars concentration (▴) is the sum of the concentrations of glucose, xylose and arabinose. The furans concentration (Δ) is the sum of the concentrations of the alcohol, aldehyde and acid derivatives of furfural and HMF. The acids concentration (□) is the sum of the concentrations of acetic and formic acid. Biomass (▪) was measured by the optical density at 600 nm.

FIG. 5 is a graphical representation of the HMF (A) and furfural (B) metabolic pathway in C. basilensis HMF14. Coloured hexamers and triangles indicate enzymes with the following activities: orange, furfural/HMF oxidoreductase; green and red, 2,5-furan-dicarboxylic acid decarboxylase; blue, 2-furoyl-CoA synthetase; yellow, furoyl-CoA dehydrogenase; purple, 2-oxoglutaryl-CoA hydrolase. Colours correspond to the genes depicted in FIG. 2A. The black square indicates a lacton hydrolysis which may occur spontaneously, or may be catalyzed by a generic lactone hydrolase. Double-pointed arrows indicate keto-enol tautomerizations. Reactions marked with (*) can be catalysed either by HmfH or by (probably non-specific) dehydrogenases.

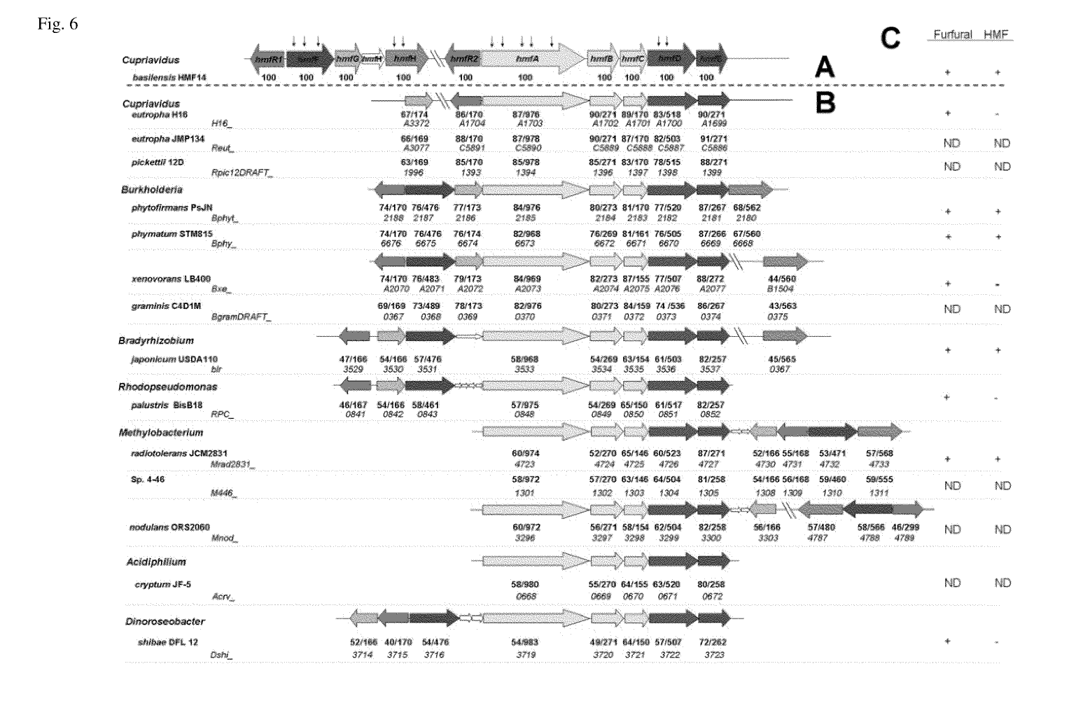

FIG. 6 shows a schematic representation of the genetic organization of the furfural and HMF metabolic genes in C. basilensis HMF14 (A) and other species (B) that were identified as potential furfural and/or HMF utilizers. Colours correspond to enzyme activities in FIG. 1. Bold numbers (x/y) below arrows indicate the percentage identity (x) to the corresponding C. basilensis HMF14 protein in a y amino-acid stretch. Orthologous genes were identified by BLASTx homology searches in the non-redundant protein database of the National Center for Biotechnology Information. Hits for the furfural cluster were defined as relevant when orthologues for hmfA, B, C, D and E were present in a single genome, with the hmfA orthologue encoding an enzyme that was at least 50% identical to HmfA. The same criterion was used to define hmfF and hmfG orthologues, whereas 40% identity to HmfH was used as the criterion for hmfH orthologues. Numbers in italics indicate genome locus tags of the indicated strain. White arrows depict genes with no metabolic function. C: Overview of growth phenotype of tested strains on mineral salts medium with either furfural or HMF (3 mM) as the sole carbon source. ND: not determined.

The following non-binding experiments illustrate the invention further.

### Culture Conditions

Cultures were performed either in Luria broth (LB) or in a minimal medium (MM). Enrichment cultures were performed in MM with 20 mM HMF as the carbon source (MMH20), supplemented as indicated with 0.1 g/l yeast extract as vitamin source (MMyH20). Solid media contained 1.5% agar. Soil and water samples were collected from the botanical garden of the Delft University of Technology and from a peat lake named ‘het Kootwijkerveen’ near Apeldoorn, The Netherlands. The samples were mixed and approximately 1 g was used to inoculate 50 ml MMH2O or MMyH2O in 500-ml Erlenmeyer flasks that were incubated at 30° C. for two days in a rotary shaker. One-ml samples were transferred twice to fresh medium and incubated until bacterial growth was apparent (OD600>1). The final enrichment cultures were streaked onto solid MMH10 and MMyH10 and incubated at 30° C. until colonies appeared.

Wheat straw hydrolysate was produced by dilute-acid hydrolysis (obtained from TNO Quality of Life, Zeist, The Netherlands). The hydrolysate was neutralized by adding 37 mM phosphate buffer (pH 7) and adjusting the pH to 7.0 with 10 M NaOH, resulting in a brown precipitate. After addition of minimal medium components, the solution was centrifuged at 10 000×g for 5 min. The supernatant was filter sterilized through a sterile PTFE filter with a pore size of 0.22 μm. The resulting medium (MMhyd) was inoculated with an overnight pre-culture of C. basilensis HMF14 in minimal medium with 3 mM HMF, 3 mM furfural and 12 mM sodium succinate.

### Bacterial Identification

Partial sequence analysis of the 16S rDNA gene was performed for preliminary identification of the bacteria isolated from the enrichment cultures. Total DNA was isolated with a FastDNA kit (QBioGene/MP Biomedicals) and the partial 16S gene was amplified by PCR using primers FD1/2, AGAGTTTGATCMTGGCTCAG and RP1/2, ACGGYTACCTTGTTACGACTT. PCR products were purified with a Qiaquick PCR purification kit (Qiagen) and sequenced by MWG Biotech AG with the same primers used for amplification.

In order to isolate the genes involved in the HMF metabolic pathway of C. basilensis HMF14, a transposon mutant library of C. basilensis HMF14 was constructed and screened for clones unable to grow on furfural and/or HMF. Twenty-five transposon mutants were selected from 14 000 clones and the chromosomal DNA flanking the transposon insertion sites was sequenced to identify the interrupted genes. Additional primer walking sequencing of up- and downstream regions of these genes revealed two distinct gene clusters. The first cluster contained five genes, designated hmfABCDE, whereas the other cluster contained four genes: hmfFGH′H. Insertion of a transposon in either of the two clusters corresponded to two distinct phenotypes. If the hmfABCDE cluster was interrupted, no growth occurred on either HMF or furfural, suggesting an—at least partly—shared degradation pathway for furfural and HMF. An insertion in the hmfFGH′H cluster resulted in loss of growth on HMF only. Mutant phenotypes of transposon mutants and BLASTx analysis {Altschul, S. F. et al. Basic local alignment search tool. J. Mol. Biol. 215, 403-410 (1990)} of the genes comprised in the two clusters are summarized in Table 1.

The resulting sequences were assigned the following functions (Table 1):

TABLE 1
Growth phenotype of selected C. basilensis HMF14 transposon mutants, and BLASTx
analysis and assigned function of genes involved in furfural and HMF degradation.

| | Growth phenotype of transposon mutant | | | | |
|---|---|---|---|---|---|
| | MM + | MM + | MM + | | Assigned |
| Gene | citrate | furfural | HMF | Best BLASTx hit (Acc. No) | function |
| hmfA (Seq. ID | + | − | − | Aerobic-type carbon | Furoyl-CoA |
| No. 36) | | | | monoxide dehydrogenase | dehydrogenase |
| | | | | homolog, subunits L and G | large |
| | | | | (YP_726196) | subunit |
| hmfB (Seq. ID | | | | Carbon-monoxide | Furoyl-CoA |
| No. 38) | | | | dehydrogenase (YP_293089) | dehydrogenase |
| | | | | | FAD binding |
| | | | | | subunit |
| hmfC (SEQ ID | | | | Aerobic-type carbon | Furoyl-CoA |
| NO: 40) | | | | monoxide dehydrogenase | dehydrogenase |
| | | | | 2Fe—2S iron-sulfur | 2Fe—2S |
| | | | | subunit (YP_726194) | iron sulfur |
| | | | | | subunit |
| hmfD (SEQ ID | + | − | − | Acyl-CoA synthetase | Furoyl-CoA |
| NO: 42) | | | | (YP_726193) | synthetase |
| hmfE (SEQ ID | | | | enoyl-CoA | 2-oxoglutaroyl- |
| NO: 44) | | | | hydratase/isomerase | CoA hydrolase |
| | | | | (YP_293086) | |
| hmfF (SEQ ID | + | + | − | UbiD family decarboxylase | 2,5-furan- |
| NO: 20) | | | | (YP_001895811) | dicarboxylic |
| | | | | | acid |
| | | | | | decarboxylase 1 |
| hmfG (SEQ ID | + | + | − | 3-octaprenyl-4- | 2,5-furan- |
| NO: 22) | | | | hydroxybenzoate carboxy- | dicarboxylic |
| | | | | lyase (ZP_02881560) | acid |
| | | | | | decarboxylase 2 |
| hmfH′ (SEQ ID | | | | hypothetical protein | NA |
| NO: 24) | | | | (YP_293096) | |
| hmfH (SEQ ID | + | + | − | glucose-methanol-choline | HMF/- |
| NO: 26) | | | | oxidoreductase | furfural |
| | | | | (YP_001895804) | oxido- |
| | | | | | reductase |
| hmfR1 (SEQ ID | | | | LysR family | Putative |
| NO: 18) | | | | transcriptional regulator | LysR type |
| | | | | (YP_001862747.1) | transcrip- |
| | | | | | t-tional |
| | | | | | regulator |
| hmfR2 (SEQ ID | | | | LysR family | Putative |
| NO: 34) | | | | transcriptional regulator | LysR type |
| | | | | (YP_293091.1) | transcrip- |
| | | | | | tional regulator |

a. The mutant phenotype was not determined since no transposon mutant was available. NA; no assigned function.

As such, a suitable host organism can preferably be transformed or transfected with DNA encoding one or more of the above sequences according to the invention.

Elucidation of the Furfural Catabolic Pathway of C. basilensis HMF14

The enzyme functions encoded by the hmfABCDE cluster of C. basilensis HMF14 were in good agreement with the enzyme activities that were reported to constitute the furoic acid degradation pathway of Pseudomonas putida strains F2 and Fu1 (see FIG. 6). The first step of this proposed pathway involves an acyl-CoA synthetase to produce 2-furoyl-CoA from 2-furoic acid, which activity matches the function of HmfD. This was supported by the accumulation of 2-furoic acid in hmfD-disrupted transposon mutants of C. basilensis HMF14 when cultured in the presence of furfuryl alcohol or furfural. Furthermore, it was established that 2-furoic acid is the substrate for ATP-dependent CoA ligation by HmfD. This activity was present in cell extracts of wildtype C. basilensis HMF14 and P. putida S12 expressing HmfD, whereas it was absent in C. basilensis HMF14 transposon mutants in which hmfD was disrupted.

In P. putida F2 and Fu1, 2-furoyl-CoA was converted into 5-hydroxy-2-furoyl-CoA by a molybdenum-dependent 2-furoyl-CoA dehydrogenase. The proteins encoded by hmfABC in C. basilensis HMF14 correspond to the three subunits that constitute a bacterial Mo-dependent dehydrogenase. The functionality of hmfABC was confirmed by demonstrating furoic-acid dependent Nitro Blue Tetrazolium (NBT) reducing activity in cell extracts of P. putida S12 co-expressing HmfABC and HmfD. The latter activity was required to generate 2-furoyl-CoA from 2-furoic acid as the substrate for HmfABC.

The HMF degradation route of C. basilensis HMF14 was reconstructed based on putative gene functions of the hmfFGH′H cluster. The hmfFG genes encode two putative decarboxylases of the UbiD/UbiX type which commonly operate concertedly 20, 21. C. basilensis HMF14 mutants with disrupted hmfFG genes accumulated HMF acid and 2,5-furan-dicarboxylic acid (FDCA) when cultured in the presence of HMF, which suggested that these carboxylic acids were the substrate for HmfFG. Cell extracts of both wildtype C. basilensis HMF14 and P. putida S12 expressing HmfFG formed 2-furoic acid when incubated with FDCA. HMF acid was not decarboxylated to furfuryl alcohol, demonstrating that FDCA was the actual substrate for HmfFG. Thus, HMF degradation in C. basilensis HMF14 proceeds obligately via its dicarboxylic acid form. No decarboxylase activity was observed in cell extract of P. putida S12 expressing HmfG only. When HmfF was expressed as a single enzyme only slight decarboxylase activity was observed, demonstrating that both proteins are required for optimal FDCA decarboxylase acitivity.

The hmfH gene encodes a putative FAD-dependent oxidoreductase.

C. basilensis HMF14 mutants with a disrupted hmfH gene accumulated HMF acid when cultured in the presence of HMF. Cell extracts of both wildtype C. basilensis HMF14 and P. putida S12 expressing HmfH formed FDCA when incubated with HMF acid, confirming that HmfH catalyzes the oxidation of the HMF-monocarboxylic acid to the dicarboxylic acid form. No FDCA was formed when oxygen was removed, demonstrating that HmfH is a true oxidase.

The hmfH′ gene encodes a hypothetical protein with 49% identity over a stretch of 296 amino acids to a probable extra-cytoplasmic solute receptor of Ralstonia eutropha H16. This gene may play a role in HMF transport, but a metabolic function was considered unlikely 22.

Analogous to the furfural pathway, no specific genes were identified for the oxidations in the upper HMF metabolic pathway leading from HMF-alcohol to HMF and HMF-acid. Also these oxidations were concluded to be performed by non-specific, redundant dehydrogenases which activities were observed both in C. basilensis HMF14 and P. putida S12 (Table 2). However, it was observed that also HmfH could oxidize HMF, furfural, and furfuryl alcohol to the corresponding acids, respectively, furfural. Apparently, this oxidase is essential for the formation of FDCA from HMF-acid but also provides an oxidase-alternative to the non-specific alcohol and aldehyde dehydrogenases that constitute the upper metabolic pathways for HMF and furfural.

Based on the above observations, the pathway depicted in FIG. 5 was constructed for HMF catabolism. First, HMF is oxidized to HMF acid, either by non-specific dehydrogenases or by HmfH. Subsequently, HMF acid is oxidized to FDCA for which conversion HmfH is essential. The HMF and the furfural catabolic pathways converge at the level of 2-furoic acid upon decarboxylation of FDCA by HmfFG.

### Analytical Methods

Bacterial growth was determined by measuring optical density at 600 nm (OD600) using a Biowave Cell Density Meter (WPA Ltd) or a μQuant MQX200 universal microplate spectrophotometer (Bio-tek), using flat-bottom 96-well microplates (Greiner). Furan derivatives were analyzed on an Agilent 1100 system equipped with a diode array detector set at 230 nm. The column used was a Zorbax Eclipse XDB-C8 (length, 150 mm; internal diameter, 4.6 mm; particle size, 5 μm; Agilent) operated at 25° C. As eluent, a gradient of acetonitrile in 20 mM KH2PO4 (pH 2) with 1% acetonitrile was used at a flow of 1.2 ml/min, increasing from 0 to 5% in 3.5 min and from 5 to 40% in 2.5 min, set as smooth gradients.

Glucose, xylose and arabinose were analyzed by ion chromatography (Dionex ICS3000 system), using a CarboPac PA20 column (length, 150 mm; internal diameter, 3 mm) with 10 mM NaOH at a flow rate of 0.5 ml·min−1 as the eluent.

For production of PHA, C. basilensis HMF14 was cultured in minimal medium with 120 mM acetate as a carbon source and 6 mM (NH4)2504 as a nitrogen source. PHA was visualized by fluorescence microscopy using Nile Blue A staining, basically as described by Johnson et al. (8)

### Chemicals

The analytical standard of 2,5-furandicarboxylic acid was purchased from Immunosource B.V. (Halle-Zoersel, Belgium). 5-Hydroxymethyl-furoic acid (HMF acid) was purchased from Matrix Scientific (Columbia S.C., United States). This compound was found to be highly esterified. Therefore, immediately prior to use, a 10 mM solution of the esterified HMF acid was boiled for two hours in 2 M H2504, cooled, and adjusted to pH 7.0 with NaOH after addition of 50 mM of phosphate buffer (pH 7). All other chemicals were purchased from Sigma-Aldrich Chemie B.V. (Zwijndrecht, The Netherlands). 5-Hydroxy-2-methylfurfuryl alcohol was identified based on its UV-VIS spectrum (3).

### Enrichment and Characterization of HMF Degrading Bacteria

Enrichment cultures on minimal medium with HMF as the sole carbon source were inoculated with soil and water samples. After 2 transfers into fresh medium, the cultures were plated on solid HMF medium to isolate individual bacteria capable of degrading HMF. Fourteen individual colonies were selected and initial identification was performed by partial 16S rDNA sequencing. Only one isolate (CB HMF14) was the only isolate incapable of utilizing glucose. In addition, HMF14 was easily culturable.

Cupriavidus sp. HMF14

Cupriavidus sp. HMF14 was able to grow on gluconate, succinate, citrate, acetate, benzene, toluene and phenol. No growth was observed on glucose, xylose, arabinose and mannose. Cells were short rods, either single, in pairs or in short chains. On LB agar plates round colonies were formed that had a mucous appearance and formation of mucus was also observed in liquid cultures. Strain HMF14 could be cultured at temperatures up to 41° C. and did not show anaerobic nitrate respiration. The phenotypic characteristics of Cupriavidus sp. HMF14 best match the type species of Cupriavidus basilensis (DSMZ 11853T). Therefore, the strain was designated Cupriavidus basilensis HMF14.

The genus Cupriavidus is well known for its ability to efficiently produce PHA (24, 31). In order to verify PHA production by the newly isolated C. basilensis HMF14, this strain was cultivated in minimal medium with acetate as a carbon source until an OD600 of 2.4 was reached. Fluorescence microscopic analysis showed PHA granules within the cells of C. basilensis (FIG. 3).

Degradation of Furan Derivatives by C. basilensis HMF14

In addition to HMF, other furan derivatives are present in lignocellulosic hydrolysates. In order to demonstrate whether C. basilensis HMF14 was capable of utilizing furan derivatives other than HMF, growth was assessed on minimal medium with 3.5 mM HMF, furfural, furfuryl alcohol or furoic acid as sole carbon source. Growth was observed on all tested furan derivatives, with slightly different growth characteristics (table 1). In cultures on furfural, furfural was initially rapidly converted to furfuryl alcohol, while also a small amount of furoic acid was formed (FIG. 1). Without wishing to be bound to any particular theory, conversion of furfural to its alcohol and/or acid form appears to be a common mechanism of furfural detoxification. At the onset of logarithmic growth, furfuryl alcohol production decreased in favour of biomass formation, which likely occurs via furoic acid. Similarly, HMF acid and -alcohol were formed in cultures with HMF as the carbon source. In addition, trace amounts of 2,5-furandicarboxylic acid and furoic acid were found in the HMF cultures (not shown).

TABLE 2
Growth characteristics of C. basilensis HMF14 on
furan derivatives.

| Carbon source | μmax (h−1) | Max. OD600 |
|---|---|---|
| HMF | 0.25^{a} | 0.95 |
| Furfural | 0.22 | 1.09 |
| Furfuryl alcohol | 0.22 | 1.08 |
| Furoic acid | 0.29 | 0.99 |

^{a}This culture did not reach stable exponential phase, since the growth rate increased continuously.

C. basilensis HMF14 grew in the presence of 5 mM of furfural or HMF (0.48 g/l, and 0.63 g/l respectively). However, since the concentration of these toxic compounds is often higher in lignocellulosic hydrolysates, with values ranging from 0 to 3.5 g/l for furfural, and from 0 to 5.9 g/l for HMF, the tolerance of C. basilensis HMF14 towards furfural and HMF was determined in shake-flask cultures with 3-15 mM furfural or HMF (FIG. 2, table 2). The lag phase was found to increase with increasing concentrations of HMF or furfural (FIG. 2), likely as a result of substrate toxicity. Nevertheless, after 24 h of cultivation growth was observed at all concentrations tested (not shown). No stable exponential phase was reached at furfural concentrations above 6 mM, but increased apparent growth rates were found at higher concentrations of furfural (results not shown). Also in the HMF cultures, no stable exponential growth was observed, but the apparent growth rate decreased rather than increased with increasing HMF concentrations.

Detoxification of Lignocellulosic Hydrolysate by C. basilensis HMF14

In addition to the furan derivatives, lignocellulosic hydrolysate also contains many other components that can inhibit fermentative production of biochemicals. While C. basilensis HMF14 is unable to degrade the sugars present in lignocellulosic hydrolysate, it preferably degrades many of the other toxic constituents, as illustrated in shake-flask cultures with each compound as a single carbon source (table 3).

TABLE 3
Degradation of constituents of lignocellulosic
hydrolysate by C. basilensis HMF14.

| | Substrate |
|---|---|
| | utilization by |
| | C. basilensis |
| Compound | HMF14^{a} |
| Sugars | |
| Glucose | N |
| Xylose | N |
| Arabinose | N |
| Mannose | N |
| Furans | Y |
| Furfural | Y |
| Furfuryl alcohol | Y |
| Hydroxymethylfurfural | Y |
| Furoic acid | |
| Organic acids | |
| Acetic acid | Y |
| Formic acid | Y^{b} |
| Levulinic acid | Y |
| Ferulic acid | Y |
| Aromatics | |
| 4-hydroxybenzoic | Y |
| acid | |
| Vanillic acid | Y |
| Syringic acid | N |
| Phenol | Y |
| 4- | Y |
| hydroxybenzaldehyde | |
| 4-hydroxybenzyl | Y |
| alcohol | |
| Guaiacol | Y |
| Vanillin | Y |
| Vanillyl alcohol | Y |
| Syringol | N |
| Syringaldehyde | N |

^{a}Y, Yes; N, No.
^{b}Formic acid was only co-utilized with a different carbon source.
This unique substrate specificity makes C. basilensis HMF14 ideally suited for the biological detoxification of lignocellulosic hydrolysate.

Heterologous Expression of the Furfural and HMF Degradation Pathways in P. putida S12

The functional characterization of the furfural and HMF catabolic genes of C. basilensis HMF14 enabled a reconstruction of the complete catabolic pathway for these furanic compounds. For a final verification of the functionality of the reconstructed pathway, the encoding genes were expressed in a heterologous host, P. putida S12.

First, the furfural cluster hmfABCDE was introduced into P. putida S12. As expected, the resulting strain, P. putida S12 pJT′hmfABCDE, was able to utilize furoic acid, furfural and furfuryl alcohol as sole carbon sources, although growth was initially poor. Therefore, strain S12 pJT′hmfABCDE was repeatedly transferred to fresh mineral salts medium with furfural as the sole carbon source. After 10 serial transfers, P. putida strain S12_fur was obtained which showed a reproducible growth rate of 0.30 h−1 on furfural as a sole carbon source with a biomass yield of 51% (C-mol biomass/C-mol substrate). P. putida S12 strains expressing only HmfABCD or HmfABC were also constructed, but these strains failed to grow on furoic acid. These results confirmed that all genes required for furfural metabolism are located in the furfural cluster hmfABCDE and that all genes in this cluster are essential for furfural metabolism, including the hmfE-encoded CoA thioester hydrolase.

Subsequently, the hmfFGH genes were cloned into P. putida S12_fur. The resulting strain, P. putida S12_HMF, was able to utilize either furfural or HMF as the sole carbon source, at a growth rate of 0.23 h−1 and a yield of 40% (C-mol biomass/C-mol substrate). Gene hmfH′ was apparently dispensable for growth on HMF, confirming that the encoded gene had no function in HMF metabolism. Nor was the gene essential for HMF transport in P. putida S12. Thus, also all genes required for the utilization of HMF were characterized, and their functionality was reconfirmed by functional expression in a heterologous host.

We claim:

1. An isolated microorganism which is Cupriavidus basilensis strain HMF14 Deposit number DSM 22875.

2. A bacterial culture comprising a Cupriavidus microorganism of Cupriavidus basilensis HMF 14 strain Deposit number DSM 22875 according to claim 1.

3. The isolated microorganism of claim 1 which, when provided with HMF, furfurylalcohol, furfural and/or furoic acid as a carbon source, grows on said source.

4. The bacterial culture of claim 2 which, when provided with HMF, furfurylalcohol, furfural and/or furoic acid as a carbon source, grows on said source.

5. The isolated microorganism of claim 3 which expresses at least one of the enzymes selected from the group consisting of furoyl-CoA dehydrogenase, furoyl-CoA synthetase, 2-oxoglutaroyl-CoA hydrolase, 2,5-furan-dicarboxylic acid decarboxylase 1,2,5-furan-dicarboxylic acid decarboxylase 2, HMF/furfural oxidoreductase, and mixtures thereof.

6. The bacterial culture of claim 4 which expresses at least one of the enzymes selected from the group consisting of furoyl-CoA dehydrogenase, furoyl-CoA synthetase, 2-oxoglutaroyl-CoA hydrolase, 2,5-furan-dicarboxylic acid decarboxylase 1,2,5-furan-dicarboxylic acid decarboxylase 2, HMF/furfural oxidoreductase, and mixtures thereof.

7. A polypeptide having aldehyde dehydrogenase activity which comprises the amino acid sequence set out in SEQ ID NO: 15 or an amino acid sequence encoded by the nucleotide sequence of SEQ ID NO: 16 or a variant polypeptide thereof, wherein the variant has at least 64% sequence identity or more with the sequence set out in SEQ ID NO: 16.

8. A polynucleotide which comprises:
(a) the nucleotide sequence set out in SEQ ID NO: 16;
(b) a nucleotide sequence which hybridizes selectively with a polynucleotide being the reverse complement of SEQ ID NO: 16;
(c) a nucleotide sequence having at least 64% sequence identity or more with the nucleotide sequence of SEQ ID NO: 16;
(d) a fragment of a nucleotide sequence as defined in (a), (b) or (c) which is at least about 100 nucleotides in length;
(e) a sequence which is degenerate as a result of the genetic code to a sequence as defined in any one of (a), (b), (c) or (d);
(f) a nucleotide sequence which is the reverse complement of a nucleotide sequence as defined in (a), (b), (c), (d) or (e).

9. A polypeptide having LysR family transcriptional regulator activity which comprises the amino acid sequence set out in SEQ ID NO: 17 or an amino acid sequence encoded by the nucleotide sequence of SEQ ID NO: 18 or a variant polypeptide thereof, wherein the variant has at least 47% sequence identity or more with the sequence set out in SEQ ID NO: 17.

10. A polynucleotide which comprises:
(a) the nucleotide sequence set out in SEQ ID NO: 18;
(b) a nucleotide sequence which hybridizes selectively with a polynucleotide being the reverse complement of SEQ ID NO: 18;
(c) a nucleotide sequence having at least 72% sequence identity or more with the nucleotide sequence of SEQ ID NO: 18;
(d) a fragment of a nucleotide sequence as defined in (a), (b) or (c) which is at least about 100 nucleotides in length;
(e) a sequence which is degenerate as a result of the genetic code to a sequence as defined in any one of (a), (b), (c) or (d);
(f) a nucleotide sequence which is the reverse complement of a nucleotide sequence as defined in (a), (b), (c), (d) or (e).

11. A polypeptide having 2,5-furan-dicarboxylic acid decarboxylase 1 activity which comprises the amino acid sequence set out in SEQ ID NO: 19 or an amino acid sequence encoded by the nucleotide sequence of SEQ ID NO: 20 or a variant polypeptide thereof, wherein the variant has at least 54% sequence identity or more with the sequence set out in SEQ ID NO: 19.

12. A polynucleotide which comprises:
(a) the nucleotide sequence set out in SEQ ID NO: 20;
(b) a nucleotide sequence which hybridizes selectively with a polynucleotide being the reverse complement of SEQ ID NO: 20;
(c) a nucleotide sequence having at least 66% sequence identity or more with the nucleotide sequence of SEQ ID NO: 20;
(d) a fragment of a nucleotide sequence as defined in (a), (b) or (c) which is at least about 100 nucleotides in length;
(e) a sequence which is degenerate as a result of the genetic code to a sequence as defined in any one of (a), (b), (c) or (d);
(f) a nucleotide sequence which is the reverse complement of a nucleotide sequence as defined in (a), (b), (c), (d) or (e).

13. A polypeptide having 2,5-furan-dicarboxylic acid decarboxylase 2 activity which comprises the amino acid sequence set out in SEQ ID NO: 21 or an amino acid sequence encoded by the nucleotide sequence of SEQ ID NO: 22 or a variant polypeptide thereof, wherein the variant has at least 52% sequence identity or more with the sequence set out in SEQ ID NO: 21.

14. A polynucleotide which comprises:
(a) the nucleotide sequence set out in SEQ ID NO: 22;
(b) a nucleotide sequence which hybridizes selectively with a polynucleotide being the reverse complement of SEQ ID NO: 22;
(c) a nucleotide sequence having at least 67% sequence identity or more with the nucleotide sequence of SEQ ID NO: 22;
(d) a fragment of a nucleotide sequence as defined in (a), (b) or (c) which is at least about 100 nucleotides in length;
(e) a sequence which is degenerate as a result of the genetic code to a sequence as defined in any one of (a), (b), (c) or (d);
(f) a nucleotide sequence which is the reverse complement of a nucleotide sequence as defined in (a), (b), (c), (d) or (e).

15. A polypeptide having HMF/furfural oxidoreductase activity which comprises the amino acid sequence set out in SEQ ID NO: 25 or an amino acid sequence encoded by the nucleotide sequence of SEQ ID NO: 26 or a variant polypeptide thereof, wherein the variant has at least 45% sequence identity or more with the sequence set out in SEQ ID NO: 25.

16. A polynucleotide which comprises:
(a) the nucleotide sequence set out in SEQ ID NO: 26;
(b) a nucleotide sequence which hybridizes selectively with a polynucleotide being the reverse complement of SEQ ID NO: 26;
(c) a nucleotide sequence having at least 66% sequence identity or more with the nucleotide sequence of SEQ ID NO: 26;
(d) a fragment of a nucleotide sequence as defined in (a), (b) or (c) which is at least about 100 nucleotides in length;
(e) a sequence which is degenerate as a result of the genetic code to a sequence as defined in any one of (a), (b), (c) or (d);
(f) a nucleotide sequence which is the reverse complement of a nucleotide sequence as defined in (a), (b), (c), (d) or (e).

17. A polypeptide having LysR type transcriptional regulator activity which comprises the amino acid sequence set out in SEQ ID NO: 33 or an amino acid sequence encoded by the nucleotide sequence of SEQ ID NO: 34 or a variant polypeptide thereof, wherein the variant has at least 46% sequence identity or more with the sequence set out in SEQ ID NO: 33.

18. A polynucleotide which comprises:
(a) the nucleotide sequence set out in SEQ ID NO: 34;
(b) a nucleotide sequence which hybridizes selectively with a polynucleotide being the reverse complement of SEQ ID NO: 34;
(c) a nucleotide sequence having at least 65% sequence identity or more with the nucleotide sequence of SEQ ID NO: 34;
(d) a fragment of a nucleotide sequence as defined in (a), (b) or (c) which is at least about 100 nucleotides in length;
(e) a sequence which is degenerate as a result of the genetic code to a sequence as defined in any one of (a), (b), (c) or (d);
(f) a nucleotide sequence which is the reverse complement of a nucleotide sequence as defined in (a), (b), (c), (d) or (e).

19. A polypeptide having Furoyl-CoA dehydrogenase (large subunit) activity which comprises the amino acid sequence set out in SEQ ID NO: 35 or an amino acid sequence encoded by the nucleotide sequence of SEQ ID NO: 36 or a variant polypeptide thereof, wherein the variant has at least 54% sequence identity or more with the sequence set out in SEQ ID NO: 35.

20. A polynucleotide which comprises:
(a) the nucleotide sequence set out in SEQ ID NO: 36;
(b) a nucleotide sequence which hybridizes selectively with a polynucleotide being the reverse complement of SEQ ID NO: 36;
(c) a nucleotide sequence having at least 66% sequence identity or more with the nucleotide sequence of SEQ ID NO: 36;
(d) a fragment of a nucleotide sequence as defined in (a), (b) or (c) which is at least about 100 nucleotides in length;
(e) a sequence which is degenerate as a result of the genetic code to a sequence as defined in any one of (a), (b), (c) or (d);
(f) a nucleotide sequence which is the reverse complement of a nucleotide sequence as defined in (a), (b), (c), (d) or (e).

21. A polypeptide having Furoyl-CoA dehydrogenase (FAD binding subunit) activity which comprises the amino acid sequence set out in SEQ ID NO: 37 or an amino acid sequence encoded by the nucleotide sequence of SEQ ID NO: 38 or a variant polypeptide thereof, wherein the variant has at least 49% sequence identity or more with the sequence set out in SEQ ID NO: 37.

22. A polynucleotide which comprises:
(a) the nucleotide sequence set out in SEQ ID NO: 38;
(b) a nucleotide sequence which hybridizes selectively with a polynucleotide being the reverse complement of SEQ ID NO: 38;
(c) a nucleotide sequence having at least 71% sequence identity or more with the nucleotide sequence of SEQ ID NO: 38;
(d) a fragment of a nucleotide sequence as defined in (a), (b) or (c) which is at least about 100 nucleotides in length;
(e) a sequence which is degenerate as a result of the genetic code to a sequence as defined in any one of (a), (b), (c) or (d);
(f) a nucleotide sequence which is the reverse complement of a nucleotide sequence as defined in (a), (b), (c), (d) or (e).

23. A polypeptide having Furoyl-CoA dehydrogenase 2Fe-2S iron sulfur subunit activity which comprises the amino acid sequence set out in SEQ ID NO: 39 or an amino acid sequence encoded by the nucleotide sequence of SEQ ID NO: 40 or a variant polypeptide thereof, wherein the variant has at least 64% sequence identity or more with the sequence set out in SEQ ID NO: 39.

24. A polynucleotide which comprises:
(a) the nucleotide sequence set out in SEQ ID NO: 40;
(b) a nucleotide sequence which hybridizes selectively with a polynucleotide being the reverse complement of SEQ ID NO: 40;
(c) a nucleotide sequence having at least 70% sequence identity or more with the nucleotide sequence of SEQ ID NO: 40; (d) a fragment of a nucleotide sequence as defined in (a), (b) or (c) which is at least about 100 nucleotides in length;
(e) a sequence which is degenerate as a result of the genetic code to a sequence as defined in any one of (a), (b), (c) or (d);
(f) a nucleotide sequence which is the reverse complement of a nucleotide sequence as defined in (a), (b), (c), (d) or (e).

25. A polypeptide having Furoyl-CoA synthetase activity which comprises the amino acid sequence set out in SEQ ID NO: 41 or an amino acid sequence encoded by the nucleotide sequence of SEQ ID NO: 42 or a variant polypeptide thereof, wherein the variant has at least 57% sequence identity or more with the sequence set out in SEQ ID NO: 41.

26. A polynucleotide which comprises:
(a) the nucleotide sequence set out in SEQ ID NO: 42;
(b) a nucleotide sequence which hybridizes selectively with a polynucleotide being the reverse complement of SEQ ID NO: 42;
(c) a nucleotide sequence having at least 68% sequence identity or more with the nucleotide sequence of SEQ ID NO: 42;
(d) a fragment of a nucleotide sequence as defined in (a), (b) or (c) which is at least about 100 nucleotides in length;
(e) a sequence which is degenerate as a result of the genetic code to a sequence as defined in any one of (a), (b), (c) or (d);
(f) a nucleotide sequence which is the reverse complement of a nucleotide sequence as defined in (a), (b), (c), (d) or (e).

27. A polypeptide having 2-oxoglutaroyl-CoA hydrolase activity which comprises the amino acid sequence set out in SEQ ID NO: 43 or an amino acid sequence encoded by the nucleotide sequence of SEQ ID NO: 44 or a variant polypeptide thereof, wherein the variant has at least 72% sequence identity or more with the sequence set out in SEQ ID NO: 43.

28. A polynucleotide which comprises:
(a) the nucleotide sequence set out in SEQ ID NO: 44;
(b) a nucleotide sequence which hybridizes selectively with a polynucleotide being the reverse complement of SEQ ID NO: 44;
(c) a nucleotide sequence having at least 74% sequence identity or more with the nucleotide sequence of SEQ ID NO: 44;
(d) a fragment of a nucleotide sequence as defined in (a), (b) or (c) which is at least about 100 nucleotides in length;
(e) a sequence which is degenerate as a result of the genetic code to a sequence as defined in any one of (a), (b), (c) or (d);
(f) a nucleotide sequence which is the reverse complement of a nucleotide sequence as defined in (a), (b), (c), (d) or (e).

29. A polynucleotide which encodes a polypeptide of claim 7.

30. A polynucleotide which encodes a polypeptide of claim 9.

31. A polynucleotide which encodes a polypeptide of claim 11.

32. A polynucleotide which encodes a polypeptide of claim 13.

33. A polynucleotide which encodes a polypeptide of claim 15.

34. A polynucleotide which encodes a polypeptide of claim 17.

35. A polynucleotide which encodes a polypeptide of claim 19.

36. A polynucleotide which encodes a polypeptide of claim 21.

37. A polynucleotide which encodes a polypeptide of claim 23.

38. A polynucleotide which encodes a polypeptide of claim 25.

39. A polynucleotide which encodes a polypeptide of claim 27.

40. A nucleic acid construct comprising the polynucleotide of claim 8.

41. A nucleic acid construct comprising the polynucleotide of claim 10.

42. A nucleic acid construct comprising the polynucleotide of claim 12.

43. A nucleic acid construct comprising the polynucleotide of claim 14.

44. A nucleic acid construct comprising the polynucleotide of claim 16.

45. A nucleic acid construct comprising the polynucleotide of claim 18.

46. A nucleic acid construct comprising the polynucleotide of claim 20.

47. A nucleic acid construct comprising the polynucleotide of claim 22.

48. A nucleic acid construct comprising the polynucleotide of claim 24.

49. A nucleic acid construct comprising the polynucleotide of claim 26.

50. A nucleic acid construct comprising the polynucleotide of claim 28.

51. A vector incorporating a polynucleotide sequence of claim 8.

52. A vector incorporating a polynucleotide sequence of claim 10.

53. A vector incorporating a polynucleotide sequence of claim 12.

54. A vector incorporating a polynucleotide sequence of claim 14.

55. A vector incorporating a polynucleotide sequence of claim 16.

56. A vector incorporating a polynucleotide sequence of claim 18.

57. A vector incorporating a polynucleotide sequence of claim 20.

58. A vector incorporating a polynucleotide sequence of claim 22.

59. A vector incorporating a polynucleotide sequence of claim 24.

60. A vector incorporating a polynucleotide sequence of claim 26.

61. A vector incorporating a polynucleotide sequence of claim 28.

62. A vector incorporating a nucleic acid construct of claim 40.

63. A vector incorporating a nucleic acid construct of claim 41.

64. A vector incorporating a nucleic acid construct of claim 42.

65. A vector incorporating a nucleic acid construct of claim 43.

66. A vector incorporating a nucleic acid construct of claim 44.

67. A vector incorporating a nucleic acid construct of claim 45.

68. A vector incorporating a nucleic acid construct of claim 46.

69. A vector incorporating a nucleic acid construct of claim 47.

70. A vector incorporating a nucleic acid construct of claim 48.

71. A vector incorporating a nucleic acid construct of claim 49.

72. A vector incorporating a nucleic acid construct of claim 50.

73. A vector incorporating a polynucleotide of claim 29.

74. A vector incorporating a polynucleotide of claim 30.

75. A vector incorporating a polynucleotide of claim 31.

76. A vector incorporating a polynucleotide of claim 32.

77. A vector incorporating a polynucleotide of claim 33.

78. A vector incorporating a polynucleotide of claim 34.

79. A vector incorporating a polynucleotide of claim 35.

80. A vector incorporating a polynucleotide of claim 36.

81. A vector incorporating a polynucleotide of claim 37.

82. A vector incorporating a polynucleotide of claim 38.

83. A vector incorporating a polynucleotide of claim 39.

84. A cell comprising a polypeptide of claim 7.

85. A cell comprising a polypeptide of claim 9.

86. A cell comprising a polypeptide of claim 11.

87. A cell comprising a polypeptide of claim 13.

88. A cell comprising a polypeptide of claim 15.

89. A cell comprising a polypeptide of claim 17.

90. A cell comprising a polypeptide of claim 19.

91. A cell comprising a polypeptide of claim 21.

92. A cell comprising a polypeptide of claim 23.

93. A cell comprising a polypeptide of claim 25.

94. A cell comprising a polypeptide of claim 27.

95. A cell comprising polynucleotide of claim 8.

96. A cell comprising polynucleotide of claim 10.

97. A cell comprising polynucleotide of claim 12.

98. A cell comprising polynucleotide of claim 14.

99. A cell comprising polynucleotide of claim 16.

100. A cell comprising polynucleotide of claim 18.

101. A cell comprising polynucleotide of claim 20.

102. A cell comprising polynucleotide of claim 22.

103. A cell comprising polynucleotide of claim 24.

104. A cell comprising polynucleotide of claim 26.

105. A cell comprising polynucleotide of claim 28.

106. A cell comprising a nucleic acid construct of claim 40.

107. A cell comprising a nucleic acid construct of claim 41.

108. A cell comprising a nucleic acid construct of claim 42.

109. A cell comprising a nucleic acid construct of claim 43.

110. A cell comprising a nucleic acid construct of claim 44.

111. A cell comprising a nucleic acid construct of claim 45.

112. A cell comprising a nucleic acid construct of claim 46.

113. A cell comprising a nucleic acid construct of claim 47.

114. A cell comprising a nucleic acid construct of claim 48.

115. A cell comprising a nucleic acid construct of claim 49.

116. A cell comprising a nucleic acid construct of claim 50.

117. A cell comprising a vector of claim 51.

118. A cell comprising a vector of claim 52.

119. A cell comprising a vector of claim 53.

120. A cell comprising a vector of claim 54.

121. A cell comprising a vector of claim 55.

122. A cell comprising a vector of claim 56.

123. A cell comprising a vector of claim 57.

124. A cell comprising a vector of claim 58.

125. A cell comprising a vector of claim 59.

126. A cell comprising a vector of claim 60.

127. A cell comprising a vector of claim 61.

128. A cell comprising a vector of claim 62.

129. A cell comprising a vector of claim 63.

130. A cell comprising a vector of claim 64.

131. A cell comprising a vector of claim 65.

132. A cell comprising a vector of claim 66.

133. A cell comprising a vector of claim 67.

134. A cell comprising a vector of claim 68.

135. A cell comprising a vector of claim 69.

136. A cell comprising a vector of claim 70.

137. A cell comprising a vector of claim 71.

138. A cell comprising a vector of claim 72.

139. A cell comprising a vector of claim 73.

140. A cell comprising a vector of claim 74.

141. A cell comprising a vector of claim 75.

142. A cell comprising a vector of claim 76.

143. A cell comprising a vector of claim 77.

144. A cell comprising a vector of claim 78.

145. A cell comprising a vector of claim 79.

146. A cell comprising a vector of claim 80.

147. A cell comprising a vector of claim 81.

148. A cell comprising a vector of claim 82.

149. A cell comprising a vector of claim 83.

150. A host microorganism cell transformed or transfected by the polynucleotide of claim 8 under conditions effective to express one or more of a Furoyl-CoA dehydrogenase, a Furoyl-CoA synthetase, a 2-oxoglutaroyl-CoA hydrolase, a 2,5-furan-dicarboxylic acid decarboxylase 1, a 2,5-furan-dicarboxylic acid decarboxylase 2 and a HMF/furfural oxidoreductase.

151. The cell of claim 150 wherein the cell is a prokaryote cell, an eukaryote cell, a plant cell or an animal cell.

152. The cell of claim 151, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

153. A host microorganism cell transformed or transfected by the polynucleotide of claim 10 under conditions effective to express one or more of a Furoyl-CoA dehydrogenase, a Furoyl-CoA synthetase, a 2-oxoglutaroyl-CoA hydrolase, a 2,5-furan-dicarboxylic acid decarboxylase 1, a 2,5-furan-dicarboxylic acid decarboxylase 2 and a HMF/furfural oxidoreductase.

154. The cell of claim 153 wherein the cell is a prokaryote cell, an eukaryote cell, a plant cell or an animal cell.

155. The cell of claim 154, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

156. A host microorganism cell transformed or transfected by the polynucleotide of claim 12 under conditions effective to express one or more of a Furoyl-CoA dehydrogenase, a Furoyl-CoA synthetase, a 2-oxoglutaroyl-CoA hydrolase, a 2,5-furan-dicarboxylic acid decarboxylase 1, a 2,5-furan-dicarboxylic acid decarboxylase 2 and a HMF/furfural oxidoreductase.

157. The cell of claim 156 wherein the cell is a prokaryote cell, an eukaryote cell, a plant cell or an animal cell.

158. The cell of claim 157, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

159. A host microorganism cell transformed or transfected by the polynucleotide of claim 14 under conditions effective to express one or more of a Furoyl-CoA dehydrogenase, a Furoyl-CoA synthetase, a 2-oxoglutaroyl-CoA hydrolase, a 2,5-furan-dicarboxylic acid decarboxylase 1, a 2,5-furan-dicarboxylic acid decarboxylase 2 and a HMF/furfural oxidoreductase.

160. The cell of claim 159 wherein the cell is a prokaryote cell, an eukaryote cell, a plant cell or an animal cell.

161. The cell of claim 160, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

162. A host microorganism cell transformed or transfected by the polynucleotide of claim 16 under conditions effective to express one or more of a Furoyl-CoA dehydrogenase, a Furoyl-CoA synthetase, a 2-oxoglutaroyl-CoA hydrolase, a 2,5-furan-dicarboxylic acid decarboxylase 1, a 2,5-furan-dicarboxylic acid decarboxylase 2 and a HMF/furfural oxidoreductase.

163. The cell of claim 162 wherein the cell is a prokaryote cell, an eukaryote cell, a plant cell or an animal cell.

164. The cell of claim 163, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

165. A host microorganism cell transformed or transfected by the polynucleotide of claim 18 under conditions effective to express one or more of a Furoyl-CoA dehydrogenase, a Furoyl-CoA synthetase, a 2-oxoglutaroyl-CoA hydrolase, a 2,5-furan-dicarboxylic acid decarboxylase 1, a 2,5-furan-dicarboxylic acid decarboxylase 2 and a HMF/furfural oxidoreductase.

166. The cell of claim 165 wherein the cell is a prokaryote cell, an eukaryote cell, a plant cell or an animal cell.

167. The cell of claim 166, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

168. A host microorganism cell transformed or transfected by the polynucleotide of claim 20 under conditions effective to express one or more of a Furoyl-CoA dehydrogenase, a Furoyl-CoA synthetase, a 2-oxoglutaroyl-CoA hydrolase, a 2,5-furan-dicarboxylic acid decarboxylase 1, a 2,5-furan-dicarboxylic acid decarboxylase 2 and a HMF/furfural oxidoreductase.

169. The cell of claim 168 wherein the cell is a prokaryote cell, an eukaryote cell, a plant cell or an animal cell.

170. The cell of claim 169, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

171. A host microorganism cell transformed or transfected by the polynucleotide of claim 22 under conditions effective to express one or more of a Furoyl-CoA dehydrogenase, a Furoyl-CoA synthetase, a 2-oxoglutaroyl-CoA hydrolase, a 2,5-furan-dicarboxylic acid decarboxylase 1, a 2,5-furan-dicarboxylic acid decarboxylase 2 and a HMF/furfural oxidoreductase.

172. The cell of claim 171 wherein the cell is a prokaryote cell, an eukaryote cell, a plant cell or an animal cell.

173. The cell of claim 172, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

174. A host microorganism cell transformed or transfected by the polynucleotide of claim 24 under conditions effective to express one or more of a Furoyl-CoA dehydrogenase, a Furoyl-CoA synthetase, a 2-oxoglutaroyl-CoA hydrolase, a 2,5-furan-dicarboxylic acid decarboxylase 1, a 2,5-furan-dicarboxylic acid decarboxylase 2 and a HMF/furfural oxidoreductase.

175. The cell of claim 174 wherein the cell is a prokaryote cell, an eukaryote cell, a plant cell or an animal cell.

176. The cell of claim 175, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

177. A host microorganism cell transformed or transfected by the polynucleotide of claim 26 under conditions effective to express one or more of a Furoyl-CoA dehydrogenase, a Furoyl-CoA synthetase, a 2-oxoglutaroyl-CoA hydrolase, a 2,5-furan-dicarboxylic acid decarboxylase 1, a 2,5-furan-dicarboxylic acid decarboxylase 2 and a HMF/furfural oxidoreductase.

178. The cell of claim 177 wherein the cell is a prokaryote cell, an eukaryote cell, a plant cell or an animal cell.

179. The cell of claim 178, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

180. A host microorganism cell transformed or transfected by the polynucleotide of claim 28 under conditions effective to express one or more of a Furoyl-CoA dehydrogenase, a Furoyl-CoA synthetase, a 2-oxoglutaroyl-CoA hydrolase, a 2,5-furan-dicarboxylic acid decarboxylase 1, a 2,5-furan-dicarboxylic acid decarboxylase 2 and a HMF/furfural oxidoreductase.

181. The cell of claim 180 wherein the cell is a prokaryote cell, an eukaryote cell, a plant cell or an animal cell.

182. The cell of claim 181, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

183. A host microorganism cell transformed or transfected by the nucleic acid construct of claim 40.

184. A host microorganism cell transformed or transfected by the nucleic acid construct of claim 41.

185. A host microorganism cell transformed or transfected by the nucleic acid construct of claim 42.

186. A host microorganism cell transformed or transfected by the nucleic acid construct of claim 43.

187. A host microorganism cell transformed or transfected by the nucleic acid construct of claim 44.

188. A host microorganism cell transformed or transfected by the nucleic acid construct of claim 45.

189. A host microorganism cell transformed or transfected by the nucleic acid construct of claim 46.

190. A host microorganism cell transformed or transfected by the nucleic acid construct of claim 47.

191. A host microorganism cell transformed or transfected by the nucleic acid construct of claim 48.

192. A host microorganism cell transformed or transfected by the nucleic acid construct of claim 49.

193. A host microorganism cell transformed or transfected by the nucleic acid construct of claim 50.

194. A host microorganism cell transformed or transfected by the vector of claim 51.

195. A host microorganism cell transformed or transfected by the vector of claim 52.

196. A host microorganism cell transformed or transfected by the vector of claim 53.

197. A host microorganism cell transformed or transfected by the vector of claim 54.

198. A host microorganism cell transformed or transfected by the vector of claim 55.

199. A host microorganism cell transformed or transfected by the vector of claim 56.

200. A host microorganism cell transformed or transfected by the vector of claim 57.

201. A host microorganism cell transformed or transfected by the vector of claim 58.

202. A host microorganism cell transformed or transfected by the vector of claim 59.

203. A host microorganism cell transformed or transfected by the vector of claim 60.

204. A host microorganism cell transformed or transfected by the vector of claim 61.

205. A host microorganism cell transformed or transfected by the vector of claim 62.

206. A host microorganism cell transformed or transfected by the vector of claim 63.

207. A host microorganism cell transformed or transfected by the vector of claim 64.

208. A host microorganism cell transformed or transfected by the vector of claim 65.

209. A host microorganism cell transformed or transfected by the vector of claim 66.

210. A host microorganism cell transformed or transfected by the vector of claim 67.

211. A host microorganism cell transformed or transfected by the vector of claim 68.

212. A host microorganism cell transformed or transfected by the vector of claim 69.

213. A host microorganism cell transformed or transfected by the vector of claim 70.

214. A host microorganism cell transformed or transfected by the vector of claim 71.

215. A host microorganism cell transformed or transfected by the vector of claim 72.

216. A host microorganism cell transformed or transfected by the vector of claim 73.

217. A host microorganism cell transformed or transfected by the vector of claim 74.

218. A host microorganism cell transformed or transfected by the vector of claim 75.

219. A host microorganism cell transformed or transfected by the vector of claim 76.

220. A host microorganism cell transformed or transfected by the vector of claim 77.

221. A host microorganism cell transformed or transfected by the vector of claim 78.

222. A host microorganism cell transformed or transfected by the vector of claim 79.

223. A host microorganism cell transformed or transfected by the vector of claim 80.

224. A host microorganism cell transformed or transfected by the vector of claim 81.

225. A host microorganism cell transformed or transfected by the vector of claim 82.

226. A host microorganism cell transformed or transfected by the vector of claim 83.

227. A host microorganism cell transformed or transfected by the nucleotide of claim 29.

228. A host microorganism cell transformed or transfected by the nucleotide of claim 30.

229. A host microorganism cell transformed or transfected by the nucleotide of claim 31.

230. A host microorganism cell transformed or transfected by the nucleotide of claim 32.

231. A host microorganism cell transformed or transfected by the nucleotide of claim 33.

232. A host microorganism cell transformed or transfected by the nucleotide of claim 34.

233. A host microorganism cell transformed or transfected by the nucleotide of claim 35.

234. A host microorganism cell transformed or transfected by the nucleotide of claim 36.

235. A host microorganism cell transformed or transfected by the nucleotide of claim 37.

236. A host microorganism cell transformed or transfected by the nucleotide of claim 38.

237. A host microorganism cell transformed or transfected by the nucleotide of claim 39.

238. The cell of claim 183 wherein the cell is a prokaryote cell, an eukaryote cell, a plant cell or an animal cell.

239. The cell of claim 184 wherein the cell is a prokaryote cell, an eukaryote cell, a plant cell or an animal cell.

240. The cell of claim 185 wherein the cell is a prokaryote cell, an eukaryote cell, a plant cell or an animal cell.

241. The cell of claim 186 wherein the cell is a prokaryote cell, an eukaryote cell, a plant cell or an animal cell.

242. The cell of claim 187 wherein the cell is a prokaryote cell, an eukaryote cell, a plant cell or an animal cell.

243. The cell of claim 188 wherein the cell is a prokaryote cell, an eukaryote cell, a plant cell or an animal cell.

244. The cell of claim 189 wherein the cell is a prokaryote cell, an eukaryote cell, a plant cell or an animal cell.

245. The cell of claim 190 wherein the cell is a prokaryote cell, an eukaryote cell, a plant cell or an animal cell.

246. The cell of claim 191 wherein the cell is a prokaryote cell, an eukaryote cell, a plant cell or an animal cell.

247. The cell of claim 192 wherein the cell is a prokaryote cell, an eukaryote cell, a plant cell or an animal cell.

248. The cell of claim 193 wherein the cell is a prokaryote cell, an eukaryote cell, a plant cell or an animal cell.

249. The cell of claim 194 wherein the cell is a prokaryote cell, an eukaryote cell, a plant cell or an animal cell.

250. The cell of claim 195 wherein the cell is a prokaryote cell, an eukaryote cell, a plant cell or an animal cell.

251. The cell of claim 196 wherein the cell is a prokaryote cell, an eukaryote cell, a plant cell or an animal cell.

252. The cell of claim 197 wherein the cell is a prokaryote cell, an eukaryote cell, a plant cell or an animal cell.

253. The cell of claim 198 wherein the cell is a prokaryote cell, an eukaryote cell, a plant cell or an animal cell.

254. The cell of claim 199 wherein the cell is a prokaryote cell, an eukaryote cell, a plant cell or an animal cell.

255. The cell of claim 200 wherein the cell is a prokaryote cell, an eukaryote cell, a plant cell or an animal cell.

256. The cell of claim 201 wherein the cell is a prokaryote cell, an eukaryote cell, a plant cell or an animal cell.

257. The cell of claim 202 wherein the cell is a prokaryote cell, an eukaryote cell, a plant cell or an animal cell.

258. The cell of claim 203 wherein the cell is a prokaryote cell, an eukaryote cell, a plant cell or an animal cell.

259. The cell of claim 204 wherein the cell is a prokaryote cell, an eukaryote cell, a plant cell or an animal cell.

260. The cell of claim 205 wherein the cell is a prokaryote cell, an eukaryote cell, a plant cell or an animal cell.

261. The cell of claim 206 wherein the cell is a prokaryote cell, an eukaryote cell, a plant cell or an animal cell.

262. The cell of claim 207 wherein the cell is a prokaryote cell, an eukaryote cell, a plant cell or an animal cell.

263. The cell of claim 208 wherein the cell is a prokaryote cell, an eukaryote cell, a plant cell or an animal cell.

264. The cell of claim 209 wherein the cell is a prokaryote cell, an eukaryote cell, a plant cell or an animal cell.

265. The cell of claim 210 wherein the cell is a prokaryote cell, an eukaryote cell, a plant cell or an animal cell.

266. The cell of claim 211 wherein the cell is a prokaryote cell, an eukaryote cell, a plant cell or an animal cell.

267. The cell of claim 212 wherein the cell is a prokaryote cell, an eukaryote cell, a plant cell or an animal cell.

268. The cell of claim 213 wherein the cell is a prokaryote cell, an eukaryote cell, a plant cell or an animal cell.

269. The cell of claim 214 wherein the cell is a prokaryote cell, an eukaryote cell, a plant cell or an animal cell.

270. The cell of claim 215 wherein the cell is a prokaryote cell, an eukaryote cell, a plant cell or an animal cell.

271. The cell of claim 216 wherein the cell is a prokaryote cell, an eukaryote cell, a plant cell or an animal cell.

272. The cell of claim 217 wherein the cell is a prokaryote cell, an eukaryote cell, a plant cell or an animal cell.

273. The cell of claim 218 wherein the cell is a prokaryote cell, an eukaryote cell, a plant cell or an animal cell.

274. The cell of claim 219 wherein the cell is a prokaryote cell, an eukaryote cell, a plant cell or an animal cell.

275. The cell of claim 220 wherein the cell is a prokaryote cell, an eukaryote cell, a plant cell or an animal cell.

276. The cell of claim 221 wherein the cell is a prokaryote cell, an eukaryote cell, a plant cell or an animal cell.

277. The cell of claim 222 wherein the cell is a prokaryote cell, an eukaryote cell, a plant cell or an animal cell.

278. The cell of claim 223 wherein the cell is a prokaryote cell, an eukaryote cell, a plant cell or an animal cell.

279. The cell of claim 224 wherein the cell is a prokaryote cell, an eukaryote cell, a plant cell or an animal cell.

280. The cell of claim 225 wherein the cell is a prokaryote cell, an eukaryote cell, a plant cell or an animal cell.

281. The cell of claim 226 wherein the cell is a prokaryote cell, an eukaryote cell, a plant cell or an animal cell.

282. The cell of claim 227 wherein the cell is a prokaryote cell, an eukaryote cell, a plant cell or an animal cell.

283. The cell of claim 228 wherein the cell is a prokaryote cell, an eukaryote cell, a plant cell or an animal cell.

284. The cell of claim 229 wherein the cell is a prokaryote cell, an eukaryote cell, a plant cell or an animal cell.

285. The cell of claim 230 wherein the cell is a prokaryote cell, an eukaryote cell, a plant cell or an animal cell.

286. The cell of claim 231 wherein the cell is a prokaryote cell, an eukaryote cell, a plant cell or an animal cell.

287. The cell of claim 232 wherein the cell is a prokaryote cell, an eukaryote cell, a plant cell or an animal cell.

288. The cell of claim 233 wherein the cell is a prokaryote cell, an eukaryote cell, a plant cell or an animal cell.

289. The cell of claim 234 wherein the cell is a prokaryote cell, an eukaryote cell, a plant cell or an animal cell.

290. The cell of claim 235 wherein the cell is a prokaryote cell, an eukaryote cell, a plant cell or an animal cell.

291. The cell of claim 236 wherein the cell is a prokaryote cell, an eukaryote cell, a plant cell or an animal cell.

292. The cell of claim 237 wherein the cell is a prokaryote cell, an eukaryote cell, a plant cell or an animal cell.

293. The cell of claim 238, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

294. The cell of claim 239, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

295. The cell of claim 240, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

296. The cell of claim 241, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

297. The cell of claim 242, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

298. The cell of claim 243, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

299. The cell of claim 244, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

300. The cell of claim 245, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

301. The cell of claim 246, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

302. The cell of claim 247, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

303. The cell of claim 248, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

304. The cell of claim 249, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

305. The cell of claim 250, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

306. The cell of claim 251, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

307. The cell of claim 252, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

308. The cell of claim 253, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

309. The cell of claim 254, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

310. The cell of claim 255, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

311. The cell of claim 256, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

312. The cell of claim 257, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

313. The cell of claim 258, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

314. The cell of claim 259, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

315. The cell of claim 260, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

316. The cell of claim 261, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

317. The cell of claim 262, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

318. The cell of claim 263, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

319. The cell of claim 264, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

320. The cell of claim 265, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

321. The cell of claim 266, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

322. The cell of claim 267, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

323. The cell of claim 268, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

324. The cell of claim 269, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

325. The cell of claim 270, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

326. The cell of claim 271, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

327. The cell of claim 272, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

328. The cell of claim 273, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

329. The cell of claim 274, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

330. The cell of claim 275, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

331. The cell of claim 276, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

332. The cell of claim 277, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

333. The cell of claim 278, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

334. The cell of claim 279, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

335. The cell of claim 280, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

336. The cell of claim 281, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

337. The cell of claim 282, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

338. The cell of claim 283, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

339. The cell of claim 284, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

340. The cell of claim 285, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

341. The cell of claim 286, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

342. The cell of claim 287, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

343. The cell of claim 288, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

344. The cell of claim 289, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

345. The cell of claim 290, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

346. The cell of claim 291, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

347. The cell of claim 292, wherein the cell is a bacterium chosen from the group of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces genus; or B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas putida, Pseudomonas putida S12, Paracoccus denitrificans, E. coli, C. glutamicum, Staphylococcus carnosus, Streptomyces lividans, Sinorhizobium melioti and Rhizobium radiobacter species.

348. A cell according to claim 151, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

349. A cell according to claim 154, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

350. A cell according to claim 157, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

351. A cell according to claim 160, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

352. A cell according to claim 163, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

353. A cell according to claim 166, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

354. A cell according to claim 169, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

355. A cell according to claim 172, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

356. A cell according to claim 175, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

357. A cell according to claim 178, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

358. A cell according to claim 181, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

359. A cell according to claim 238, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

360. A cell according to claim 239, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

361. A cell according to claim 240, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

362. A cell according to claim 241, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

363. A cell according to claim 242, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

364. A cell according to claim 243, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

365. A cell according to claim 244, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

366. A cell according to claim 245, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

367. A cell according to claim 246, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

368. A cell according to claim 247, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

369. A cell according to claim 248, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

370. A cell according to claim 249, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

371. A cell according to claim 250, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

372. A cell according to claim 251, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

373. A cell according to claim 252, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

374. A cell according to claim 253, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

375. A cell according to claim 254, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

376. A cell according to claim 255, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

377. A cell according to claim 256, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

378. A cell according to claim 257, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

379. A cell according to claim 258, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

380. A cell according to claim 259, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

381. A cell according to claim 260, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

382. A cell according to claim 261, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

383. A cell according to claim 262, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

384. A cell according to claim 263, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

385. A cell according to claim 264, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

386. A cell according to claim 265, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

387. A cell according to claim 266, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

388. A cell according to claim 267, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

389. A cell according to claim 268, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

390. A cell according to claim 269, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

391. A cell according to claim 270, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

392. A cell according to claim 271, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

393. A cell according to claim 272, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

394. A cell according to claim 273, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

395. A cell according to claim 274, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

396. A cell according to claim 275, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

397. A cell according to claim 276, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

398. A cell according to claim 277, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

399. A cell according to claim 278, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

400. A cell according to claim 279, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

401. A cell according to claim 280, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

402. A cell according to claim 281, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

403. A cell according to claim 282, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

404. A cell according to claim 283, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

405. A cell according to claim 284, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

406. A cell according to claim 285, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

407. A cell according to claim 286, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

408. A cell according to claim 287, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

409. A cell according to claim 288, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

410. A cell according to claim 289, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

411. A cell according to claim 290, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

412. A cell according to claim 291, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

413. A cell according to claim 292, wherein the cell is a yeast cell from the group consisting of the Candida, Hansenula, Kluyveromyces, Pichia, Saccharomyces, Schizosaccharomyces, or Yarrowia genus; or Kluyveromyces lactis, S. cerevisiae, Hansenula polymorpha, Yarrowia lipolytica and Pichia pastoris species or a filamentous fungal cell from the group Aspergillus, Chrysosporium, Penicillium, Talaromyces or Trichoderma genus; or Aspergillus niger, Aspergillus awamori, Aspergillus foetidus, Aspergillus sojae, Aspergillus fumigatus, Talaromyces emersonii, Aspergillus oryzae, Chrysosporium lucknowense, Trichoderma reesei or Penicillium chrysogenum species.

414. The cell of claim 155 wherein one or more gene is deleted, knocked-out or disrupted in full or in part, wherein optionally the gene encodes for a protease.

415. The cell of claim 152 wherein one or more gene is deleted, knocked-out or disrupted in full or in part, wherein optionally the gene encodes for a protease.

416. The cell of claim 158 wherein one or more gene is deleted, knocked-out or disrupted in full or in part, wherein optionally the gene encodes for a protease.

417. The cell of claim 161 wherein one or more gene is deleted, knocked-out or disrupted in full or in part, wherein optionally the gene encodes for a protease.

418. The cell of claim 164 wherein one or more gene is deleted, knocked-out or disrupted in full or in part, wherein optionally the gene encodes for a protease.

419. The cell of claim 167 wherein one or more gene is deleted, knocked-out or disrupted in full or in part, wherein optionally the gene encodes for a protease.

420. The cell of claim 170 wherein one or more gene is deleted, knocked-out or disrupted in full or in part, wherein optionally the gene encodes for a protease.

421. The cell of claim 173 wherein one or more gene is deleted, knocked-out or disrupted in full or in part, wherein optionally the gene encodes for a protease.

422. The cell of claim 176 wherein one or more gene is deleted, knocked-out or disrupted in full or in part, wherein optionally the gene encodes for a protease.

423. The cell of claim 179 wherein one or more gene is deleted, knocked-out or disrupted in full or in part, wherein optionally the gene encodes for a protease.

424. The cell of claim 182 wherein one or more gene is deleted, knocked-out or disrupted in full or in part, wherein optionally the gene encodes for a protease.

425. The cell of claim 293 wherein one or more gene is deleted, knocked-out or disrupted in full or in part, wherein optionally the gene encodes for a protease.

426. The cell of claim 294 wherein one or more gene is deleted, knocked-out or disrupted in full or in part, wherein optionally the gene encodes for a protease.

427. The cell of claim 295 wherein one or more gene is deleted, knocked-out or disrupted in full or in part, wherein optionally the gene encodes for a protease.

428. The cell of claim 296 wherein one or more gene is deleted, knocked-out or disrupted in full or in part, wherein optionally the gene encodes for a protease.

429. The cell of claim 297 wherein one or more gene is deleted, knocked-out or disrupted in full or in part, wherein optionally the gene encodes for a protease.

430. The cell of claim 298 wherein one or more gene is deleted, knocked-out or disrupted in full or in part, wherein optionally the gene encodes for a protease.

431. The cell of claim 299 wherein one or more gene is deleted, knocked-out or disrupted in full or in part, wherein optionally the gene encodes for a protease.

432. The cell of claim 300 wherein one or more gene is deleted, knocked-out or disrupted in full or in part, wherein optionally the gene encodes for a protease.

433. The cell of claim 301 wherein one or more gene is deleted, knocked-out or disrupted in full or in part, wherein optionally the gene encodes for a protease.

434. The cell of claim 302 wherein one or more gene is deleted, knocked-out or disrupted in full or in part, wherein optionally the gene encodes for a protease.

435. The cell of claim 303 wherein one or more gene is deleted, knocked-out or disrupted in full or in part, wherein optionally the gene encodes for a protease.

436. The cell of claim 304 wherein one or more gene is deleted, knocked-out or disrupted in full or in part, wherein optionally the gene encodes for a protease.

437. The cell of claim 305 wherein one or more gene is deleted, knocked-out or disrupted in full or in part, wherein optionally the gene encodes for a protease.

438. The cell of claim 306 wherein one or more gene is deleted, knocked-out or disrupted in full or in part, wherein optionally the gene encodes for a protease.

439. The cell of claim 307 wherein one or more gene is deleted, knocked-out or disrupted in full or in part, wherein optionally the gene encodes for a protease.

440. The cell of claim 308 wherein one or more gene is deleted, knocked-out or disrupted in full or in part, wherein optionally the gene encodes for a protease.

441. The cell of claim 309 wherein one or more gene is deleted, knocked-out or disrupted in full or in part, wherein optionally the gene encodes for a protease.

442. The cell of claim 310 wherein one or more gene is deleted, knocked-out or disrupted in full or in part, wherein optionally the gene encodes for a protease.

443. The cell of claim 311 wherein one or more gene is deleted, knocked-out or disrupted in full or in part, wherein optionally the gene encodes for a protease.

444. The cell of claim 312 wherein one or more gene is deleted, knocked-out or disrupted in full or in part, wherein optionally the gene encodes for a protease.

445. The cell of claim 313 wherein one or more gene is deleted, knocked-out or disrupted in full or in part, wherein optionally the gene encodes for a protease.

446. The cell of claim 314 wherein one or more gene is deleted, knocked-out or disrupted in full or in part, wherein optionally the gene encodes for a protease.

447. The cell of claim 315 wherein one or more gene is deleted, knocked-out or disrupted in full or in part, wherein optionally the gene encodes for a protease.

448. The cell of claim 316 wherein one or more gene is deleted, knocked-out or disrupted in full or in part, wherein optionally the gene encodes for a protease.

449. The cell of claim 317 wherein one or more gene is deleted, knocked-out or disrupted in full or in part, wherein optionally the gene encodes for a protease.

450. The cell of claim 318 wherein one or more gene is deleted, knocked-out or disrupted in full or in part, wherein optionally the gene encodes for a protease.

451. The cell of claim 319 wherein one or more gene is deleted, knocked-out or disrupted in full or in part, wherein optionally the gene encodes for a protease.

452. The cell of claim 320 wherein one or more gene is deleted, knocked-out or disrupted in full or in part, wherein optionally the gene encodes for a protease.

453. The cell of claim 321 wherein one or more gene is deleted, knocked-out or disrupted in full or in part, wherein optionally the gene encodes for a protease.

454. The cell of claim 322 wherein one or more gene is deleted, knocked-out or disrupted in full or in part, wherein optionally the gene encodes for a protease.

455. The cell of claim 323 wherein one or more gene is deleted, knocked-out or disrupted in full or in part, wherein optionally the gene encodes for a protease.

456. The cell of claim 324 wherein one or more gene is deleted, knocked-out or disrupted in full or in part, wherein optionally the gene encodes for a protease.

457. The cell of claim 325 wherein one or more gene is deleted, knocked-out or disrupted in full or in part, wherein optionally the gene encodes for a protease.

458. The cell of claim 326 wherein one or more gene is deleted, knocked-out or disrupted in full or in part, wherein optionally the gene encodes for a protease.

459. The cell of claim 327 wherein one or more gene is deleted, knocked-out or disrupted in full or in part, wherein optionally the gene encodes for a protease.

460. The cell of claim 328 wherein one or more gene is deleted, knocked-out or disrupted in full or in part, wherein optionally the gene encodes for a protease.

461. The cell of claim 329 wherein one or more gene is deleted, knocked-out or disrupted in full or in part, wherein optionally the gene encodes for a protease.

462. The cell of claim 330 wherein one or more gene is deleted, knocked-out or disrupted in full or in part, wherein optionally the gene encodes for a protease.

463. The cell of claim 331 wherein one or more gene is deleted, knocked-out or disrupted in full or in part, wherein optionally the gene encodes for a protease.

464. The cell of claim 332 wherein one or more gene is deleted, knocked-out or disrupted in full or in part, wherein optionally the gene encodes for a protease.

465. The cell of claim 333 wherein one or more gene is deleted, knocked-out or disrupted in full or in part, wherein optionally the gene encodes for a protease.

466. The cell of claim 334 wherein one or more gene is deleted, knocked-out or disrupted in full or in part, wherein optionally the gene encodes for a protease.

467. The cell of claim 335 wherein one or more gene is deleted, knocked-out or disrupted in full or in part, wherein optionally the gene encodes for a protease.

468. The cell of claim 336 wherein one or more gene is deleted, knocked-out or disrupted in full or in part, wherein optionally the gene encodes for a protease.

469. The cell of claim 337 wherein one or more gene is deleted, knocked-out or disrupted in full or in part, wherein optionally the gene encodes for a protease.

470. The cell of claim 338 wherein one or more gene is deleted, knocked-out or disrupted in full or in part, wherein optionally the gene encodes for a protease.

471. The cell of claim 339 wherein one or more gene is deleted, knocked-out or disrupted in full or in part, wherein optionally the gene encodes for a protease.

472. The cell of claim 340 wherein one or more gene is deleted, knocked-out or disrupted in full or in part, wherein optionally the gene encodes for a protease.

473. The cell of claim 341 wherein one or more gene is deleted, knocked-out or disrupted in full or in part, wherein optionally the gene encodes for a protease.

474. The cell of claim 342 wherein one or more gene is deleted, knocked-out or disrupted in full or in part, wherein optionally the gene encodes for a protease.

475. The cell of claim 343 wherein one or more gene is deleted, knocked-out or disrupted in full or in part, wherein optionally the gene encodes for a protease.

476. The cell of claim 344 wherein one or more gene is deleted, knocked-out or disrupted in full or in part, wherein optionally the gene encodes for a protease.

477. The cell of claim 345 wherein one or more gene is deleted, knocked-out or disrupted in full or in part, wherein optionally the gene encodes for a protease.

478. The cell of claim 346 wherein one or more gene is deleted, knocked-out or disrupted in full or in part, wherein optionally the gene encodes for a protease.

479. The cell of claim 347 wherein one or more gene is deleted, knocked-out or disrupted in full or in part, wherein optionally the gene encodes for a protease.

480. The cell of claim 348 wherein one or more gene is deleted, knocked-out or disrupted in full or in part, wherein optionally the gene encodes for a protease.

481. The cell of claim 349 wherein one or more gene is deleted, knocked-out or disrupted in full or in part, wherein optionally the gene encodes for a protease.

482. The cell of claim 350 wherein one or more gene is deleted, knocked-out or disrupted in full or in part, wherein optionally the gene encodes for a protease.

483. The cell of claim 351 wherein one or more gene is deleted, knocked-out or disrupted in full or in part, wherein optionally the gene encodes for a protease.

484. The cell of claim 352 wherein one or more gene is deleted, knocked-out or disrupted in full or in part, wherein optionally the gene encodes for a protease.

485. The cell of claim 353 wherein one or more gene is deleted, knocked-out or disrupted in full or in part, wherein optionally the gene encodes for a protease.

486. The cell of claim 354 wherein one or more gene is deleted, knocked-out or disrupted in full or in part, wherein optionally the gene encodes for a protease.

487. The cell of claim 355 wherein one or more gene is deleted, knocked-out or disrupted in full or in part, wherein optionally the gene encodes for a protease.

488. The cell of claim 356 wherein one or more gene is deleted, knocked-out or disrupted in full or in part, wherein optionally the gene encodes for a protease.

489. The cell of claim 357 wherein one or more gene is deleted, knocked-out or disrupted in full or in part, wherein optionally the gene encodes for a protease.

490. The cell of claim 358 wherein one or more gene is deleted, knocked-out or disrupted in full or in part, wherein optionally the gene encodes for a protease.

491. The cell of claim 359 wherein one or more gene is deleted, knocked-out or disrupted in full or in part, wherein optionally the gene encodes for a protease.

492. The cell of claim 360 wherein one or more gene is deleted, knocked-out or disrupted in full or in part, wherein optionally the gene encodes for a protease.

493. The cell of claim 361 wherein one or more gene is deleted, knocked-out or disrupted in full or in part, wherein optionally the gene encodes for a protease.

494. The cell of claim 362 wherein one or more gene is deleted, knocked-out or disrupted in full or in part, wherein optionally the gene encodes for a protease.

495. The cell of claim 363 wherein one or more gene is deleted, knocked-out or disrupted in full or in part, wherein optionally the gene encodes for a protease.

496. The cell of claim 364 wherein one or more gene is deleted, knocked-out or disrupted in full or in part, wherein optionally the gene encodes for a protease.

497. The cell of claim 365 wherein one or more gene is deleted, knocked-out or disrupted in full or in part, wherein optionally the gene encodes for a protease.

498. The cell of claim 366 wherein one or more gene is deleted, knocked-out or disrupted in full or in part, wherein optionally the gene encodes for a protease.

499. The cell of claim 367 wherein one or more gene is deleted, knocked-out or disrupted in full or in part, wherein optionally the gene encodes for a protease.

500. The cell of claim 368 wherein one or more gene is deleted, knocked-out or disrupted in full or in part, wherein optionally the gene encodes for a protease.

501. The cell of claim 369 wherein one or more gene is deleted, knocked-out or disrupted in full or in part, wherein optionally the gene encodes for a protease.

502. The cell of claim 370 wherein one or more gene is deleted, knocked-out or disrupted in full or in part, wherein optionally the gene encodes for a protease.

503. The cell of claim 371 wherein one or more gene is deleted, knocked-out or disrupted in full or in part, wherein optionally the gene encodes for a protease.

504. The cell of claim 372 wherein one or more gene is deleted, knocked-out or disrupted in full or in part, wherein optionally the gene encodes for a protease.

505. The cell of claim 373 wherein one or more gene is deleted, knocked-out or disrupted in full or in part, wherein optionally the gene encodes for a protease.

506. The cell of claim 374 wherein one or more gene is deleted, knocked-out or disrupted in full or in part, wherein optionally the gene encodes for a protease.

507. The cell of claim 375 wherein one or more gene is deleted, knocked-out or disrupted in full or in part, wherein optionally the gene encodes for a protease.

508. The cell of claim 376 wherein one or more gene is deleted, knocked-out or disrupted in full or in part, wherein optionally the gene encodes for a protease.

509. The cell of claim 377 wherein one or more gene is deleted, knocked-out or disrupted in full or in part, wherein optionally the gene encodes for a protease.

510. The cell of claim 378 wherein one or more gene is deleted, knocked-out or disrupted in full or in part, wherein optionally the gene encodes for a protease.

511. The cell of claim 379 wherein one or more gene is deleted, knocked-out or disrupted in full or in part, wherein optionally the gene encodes for a protease.

512. The cell of claim 380 wherein one or more gene is deleted, knocked-out or disrupted in full or in part, wherein optionally the gene encodes for a protease.

513. The cell of claim 381 wherein one or more gene is deleted, knocked-out or disrupted in full or in part, wherein optionally the gene encodes for a protease.

514. The cell of claim 382 wherein one or more gene is deleted, knocked-out or disrupted in full or in part, wherein optionally the gene encodes for a protease.

515. The cell of claim 383 wherein one or more gene is deleted, knocked-out or disrupted in full or in part, wherein optionally the gene encodes for a protease.

516. The cell of claim 384 wherein one or more gene is deleted, knocked-out or disrupted in full or in part, wherein optionally the gene encodes for a protease.

517. The cell of claim 385 wherein one or more gene is deleted, knocked-out or disrupted in full or in part, wherein optionally the gene encodes for a protease.

518. The cell of claim 386 wherein one or more gene is deleted, knocked-out or disrupted in full or in part, wherein optionally the gene encodes for a protease.

519. The cell of claim 387 wherein one or more gene is deleted, knocked-out or disrupted in full or in part, wherein optionally the gene encodes for a protease.

520. The cell of claim 388 wherein one or more gene is deleted, knocked-out or disrupted in full or in part, wherein optionally the gene encodes for a protease.

521. The cell of claim 389 wherein one or more gene is deleted, knocked-out or disrupted in full or in part, wherein optionally the gene encodes for a protease.

522. The cell of claim 390 wherein one or more gene is deleted, knocked-out or disrupted in full or in part, wherein optionally the gene encodes for a protease.

523. The cell of claim 391 wherein one or more gene is deleted, knocked-out or disrupted in full or in part, wherein optionally the gene encodes for a protease.

524. The cell of claim 392 wherein one or more gene is deleted, knocked-out or disrupted in full or in part, wherein optionally the gene encodes for a protease.

525. The cell of claim 393 wherein one or more gene is deleted, knocked-out or disrupted in full or in part, wherein optionally the gene encodes for a protease.

526. The cell of claim 394 wherein one or more gene is deleted, knocked-out or disrupted in full or in part, wherein optionally the gene encodes for a protease.

527. The cell of claim 395 wherein one or more gene is deleted, knocked-out or disrupted in full or in part, wherein optionally the gene encodes for a protease.

528. The cell of claim 396 wherein one or more gene is deleted, knocked-out or disrupted in full or in part, wherein optionally the gene encodes for a protease.

529. The cell of claim 397 wherein one or more gene is deleted, knocked-out or disrupted in full or in part, wherein optionally the gene encodes for a protease.

530. The cell of claim 398 wherein one or more gene is deleted, knocked-out or disrupted in full or in part, wherein optionally the gene encodes for a protease.

531. The cell of claim 399 wherein one or more gene is deleted, knocked-out or disrupted in full or in part, wherein optionally the gene encodes for a protease.

532. The cell of claim 400 wherein one or more gene is deleted, knocked-out or disrupted in full or in part, wherein optionally the gene encodes for a protease.

533. The cell of claim 401 wherein one or more gene is deleted, knocked-out or disrupted in full or in part, wherein optionally the gene encodes for a protease.

534. The cell of claim 402 wherein one or more gene is deleted, knocked-out or disrupted in full or in part, wherein optionally the gene encodes for a protease.

535. The cell of claim 403 wherein one or more gene is deleted, knocked-out or disrupted in full or in part, wherein optionally the gene encodes for a protease.

536. The cell of claim 404 wherein one or more gene is deleted, knocked-out or disrupted in full or in part, wherein optionally the gene encodes for a protease.

537. The cell of claim 405 wherein one or more gene is deleted, knocked-out or disrupted in full or in part, wherein optionally the gene encodes for a protease.

538. The cell of claim 406 wherein one or more gene is deleted, knocked-out or disrupted in full or in part, wherein optionally the gene encodes for a protease.

539. The cell of claim 407 wherein one or more gene is deleted, knocked-out or disrupted in full or in part, wherein optionally the gene encodes for a protease.

540. The cell of claim 408 wherein one or more gene is deleted, knocked-out or disrupted in full or in part, wherein optionally the gene encodes for a protease.

541. The cell of claim 409 wherein one or more gene is deleted, knocked-out or disrupted in full or in part, wherein optionally the gene encodes for a protease.

542. The cell of claim 410 wherein one or more gene is deleted, knocked-out or disrupted in full or in part, wherein optionally the gene encodes for a protease.

543. The cell of claim 411 wherein one or more gene is deleted, knocked-out or disrupted in full or in part, wherein optionally the gene encodes for a protease.

544. The cell of claim 412 wherein one or more gene is deleted, knocked-out or disrupted in full or in part, wherein optionally the gene encodes for a protease.

545. The cell of claim 413 wherein one or more gene is deleted, knocked-out or disrupted in full or in part, wherein optionally the gene encodes for a protease.

546. A cell extract from a Cupriavidus microorganism of claim 1 comprising a Furoyl-CoA dehydrogenase and/or a Furoyl-CoA synthetase and/or a 2-oxoglutaroyl-CoA hydrolase and/or a 2,5-furan-dicarboxylic acid decarboxylase 1 and/or a 2,5-furan-dicarboxylic acid decarboxylase 2 and/or a HMF/furfural oxidoreductase.

547. A cell extract from a Cupriavidus microorganism of claim 2 comprising a Furoyl-CoA dehydrogenase and/or a Furoyl-CoA synthetase and/or a 2-oxoglutaroyl-CoA hydrolase and/or a 2,5-furan-dicarboxylic acid decarboxylase 1 and/or a 2,5-furan-dicarboxylic acid decarboxylase 2 and/or a HMF/furfural oxidoreductase.

548. A cell extract from a host cell of claim 151, 152, 154, 155, 157, 158, 160, 161, 163, 164, 166, 167, 169, 170, 172, 173, 175, 176, 178, 179, 181, 182, 238-413 comprising a Furoyl-CoA dehydrogenase and/or a Furoyl-CoA synthetase and/or a 2-oxoglutaroyl-CoA hydrolase and/or a 2,5-furan-dicarboxylic acid decarboxylase 1 and/or a 2,5-furan-dicarboxylic acid decarboxylase 2 and/or a HMF/furfural oxidoreductase.

549. A process for the in-situ detoxification of a lignocellulose hydrolysate containing furanic compounds, such as preferably one or more of HMF, Furfurylalcohol, Furfural and/or Furoic acid, with a suitable host microorganism, the process comprising a host microorganism from the family of Burkholderiaceae and contacting the lignocellulose hydrolysate with the host microorganism under conditions facilitating the expression of one or more of a Furoyl-CoA dehydrogenase, a Furoyl-CoA synthetase, a 2-oxoglutaroyl-CoA hydrolase, a 2,5-furan-dicarboxylic acid decarboxylase 1, a 2,5-furan-dicarboxylic acid decarboxylase 2 and a HMF/furfural oxidoreductase from the host microorganism from the family of Burkholderiaceae to convert furanic compounds, such as HMF, Furfurylalcohol, Furfural and/or Furoic acid, to non-toxic components to obtain a detoxified lignocellulose hydrolysate.

550. A process of claim 549 wherein the Cupriavidus microorganism is Cupriavidus basilensis.

551. The process of claim 550 wherein the Cupriavidus microorganism is Cupriavidus basilensis strain HMF14 deposit number DSM 22875.

552. The process of claim 549 further comprising subjecting the detoxified lignocellulose hydrolysate to a simultaneous or subsequent fermentation step.

553. The process of claim 549 further comprising a step of pre-treating lignocellulose-containing material to obtain a lignocellulose hydrolysate, preferably under acidic conditions.

554. The process of claim 549 comprising introducing (a) a polynucleotide which encodes a polypeptide having aldehyde dehydrogenase activity which comprises the amino acid sequence set out in SEQ ID NO: 15 or an amino acid sequence encoded by the nucleotide sequence of SEQ ID NO: 16 or a variant polypeptide thereof, wherein the variant has at least 64% sequence identity or more with the sequence set out in SEQ ID NO: 16 in an appropriate host cell, cultivating the obtained host cell under conditions conducive to the detoxification of lignocelluloses hydrolysate, and recovering a detoxified lignocelluloses hydrolysate from the culture.

555. A process for the production of Furoyl-CoA dehydrogenase and/or Furoyl-CoA synthetase and/or 2-oxoglutaroyl-CoA hydrolase and/or 2,5-furan-dicarboxylic acid decarboxylase 1 and/or 2,5-furan-dicarboxylic acid decarboxylase 2 and/or a HMF/furfural oxidoreductase which are at least 45% identical to those expressed by Cupriavidus basilensis HMF14 DSM 22875, comprising
(a) culturing a microorganism in a nutrient medium containing carbon and nitrogen sources and inorganic salts; and
(b) isolating the enzymes produced from the microorganism.

556. A process for the conversion of 5-hydroxymethylfurfural (HMF), 2,5-dihydroxymethyl furan (HMF alcohol), 5-hydroxymethyl-2-furancarboxylic acid (HMF acid) and/or 2,5-furandicarboxylic acid to 2-furoyl CoA, comprising contacting furfuryl alcohol and/or furfural with a furoyl-CoA dehydrogenase, a furoyl-CoA synthetase, a 2-oxoglutaroyl-CoA hydrolase, and a 2,5-furan-dicarboxylic acid decarboxylase catalyst in the presence of one or more coenzyme cofactor(s).

557. The process of claim 556 wherein the catalyst comprises a polypeptide having 2,5-furan-dicarboxylic acid decarboxylase 1 activity which comprises the amino acid sequence set out in SEQ ID NO: 19 or an amino acid sequence encoded by the nucleotide sequence of SEQ ID NO: 20 or a variant polypeptide thereof, wherein the variant has at least 54% sequence identity or more with the sequence set out in SEQ ID NO: 19.

558. A method for the biocatalytic production of biofuels such as ethanol in a suitable host microorganism of the enzymatic pathway of Cupriavidus basilensis strain DSM 22875, under conditions facilitating the expression of the activity of the enzymes in the enzymatic pathway.

559. A process for the production of second-generation biofuels from a lignocelluloses-containing material, which process comprises
a) pretreating and hydrolysis of the lignocellulose-containing material to prepare a lignocellulosic hydrolysate comprising one or more furanic compounds and one or more fermentable sugars;
b) in-situ detoxification of the lignocellulose hydrolysate comprising contacting the lignocellulose hydrolysate with a host microorganism under conditions facilitating the expression of one or more of a Furoyl-CoA dehydrogenase, a Furoyl-CoA synthetase, a 2-oxoglutaroyl-CoA hydrolase, a 2,5-furan-dicarboxylic acid decarboxylase 1, a 2,5-furan-dicarboxylic acid decarboxylase 2 and/or a HMF/furfural oxidoreductase from the host microorganism to convert the one or more furanic compounds to non-toxic components to obtain a detoxified lignocellulose hydrolysate;
c) conversion of the fermentable sugars from the lignocellulose hydrolysate or the detoxified lignocelluloses hydrolysate to produce a biofuel.

560. The process of claim 559 wherein the one or more furanic compounds are selected from the group consisting of furfural, furfuryl alcohol, 5-(hydroxymethyl)-furfural (HMF) and furoic acid.

561. The process of claim 559 wherein the host microorganism is a host microorganism from the family of Burkholderiaceae.

562. The process of claim 559 wherein the conversion of fermentable sugars in step c) comprises fermentation of the fermentable sugars.

563. The process of claim 559 wherein in step b) a stable fermentable sugar concentration is maintained before all furanic compounds are converted.

564. The process of claim 559 further comprising the step of catalytic cracking and/or hydrotreating of the biofuel to prepare a cracked and/or hydrotreated biofuel.

565. The process of claim 559 further comprising the step of blending the, optionally cracked or hydrotreated, biofuel with and one or more other components to produce an automotive fuel.


SEQUENCE LISTING (46 sequences)

SEQ ID NO: 1 - DNA, 20 bp, Artificial Sequence
  Primer
gcacgcgcctgagttacgac

SEQ ID NO: 2 - DNA, 19 bp, Artificial Sequence
  Primer
catgctcggcgctggtgac

SEQ ID NO: 3 - DNA, 29 bp, Artificial Sequence
  Primer
catgaattccgacccaggagtcacgccat

SEQ ID NO: 4 - DNA, 36 bp, Artificial Sequence
  Primer
cggcggccgcggatataccgacaatgatgcgtctct

SEQ ID NO: 5 - DNA, 34 bp, Artificial Sequence
  Primer
cggcggccgctgtctcctgcctgttcagcattca

SEQ ID NO: 6 - DNA, 33 bp, Artificial Sequence
  Primer
gcgggccccttactccttgatggtatcgacagg

SEQ ID NO: 7 - DNA, 26 bp, Artificial Sequence
  Primer
gcggtaccgggagggccggtcatgag

SEQ ID NO: 8 - DNA, 28 bp, Artificial Sequence
  Primer
gcggtaccggcgtagatacccaggaggc

SEQ ID NO: 9 - DNA, 28 bp, Artificial Sequence
  Primer
gcgggcccccacgctttgcaggaaggtg

SEQ ID NO: 10 - DNA, 26 bp, Artificial Sequence
  Primer
cggaattccggcgcatgtgttcacgc

SEQ ID NO: 11 - DNA, 29 bp, Artificial Sequence
  Primer
gcgcggccgccgcccgcgatttccatcag

SEQ ID NO: 12 - DNA, 32 bp, Artificial Sequence
  Primer
gcggtaccccatcaaggagtaagacatgaccc

SEQ ID NO: 13 - DNA, 28 bp, Artificial Sequence
  Primer
cggaattccacatgacaaggggagaccg

SEQ ID NO: 14 - DNA, 30 bp, Artificial Sequence
  Primer
cggaattcgcttcggtcttcaactcggatg

SEQ ID NO: 15 - protein, 500 aa, Cupriavidus basilensis
  misc_feature: Aldehyde Dehydrogenase, HMF 14
MNAQHWIAGAWTGEPSADSVNPADGTLIGQFADGGTWQAEAAIAAARHVFERTTWGQDARLRQDVLLAWAGALEAERERLASLLTAENGKPVAQARGEVGAAISEVRYYAGLARHIPGHVLEPEPGTISTILREPAGVAAIIVPWNAPAVLLVRSLAPALAAGCTAVVKSAAQTTLFTAAMLRLFERTALPAGAVNLVCETGYAAADHLVRSRDVDVVSFTGSTATGKKIMIAAADSVKKLSLELGGKSCCLVFDDVDAQAVAKRLALAATVISGQQCTAARRVLVHEAIAPQMRRHLTEALAALRLGPGIEPDTQIGPLIDHPTRAMVSAQVERACDEADTVLLRGTMPGGALARGAFLSPTLVEHSDPGAFFCQEEIFGPFVTFETFATEDEALAKANNTVFGLSASVWTHHGERAIRLARALRNGTVWVNDHNRLFAEAETGGYRQSGLGRLHGYDALADFTELKHICIQAGLPKGMSQAGCRLSGVAARERMGVSV

SEQ ID NO: 16 - DNA, 1503 bp, Cupriavidus basilensis
  misc_feature: Aldehyde Dehydrogenase, HMF 14
atgaacgcgcaacactggattgccggcgcctggaccggcgagccttccgccgatagcgtcaaccccgccgacgggaccctgatcgggcagttcgcggacggcggcacctggcaagccgaagccgccatcgccgccgcgcgccatgtcttcgagcgcaccacctggggccaggatgcccgcctgcgccaggacgtgcttctagcctgggctggtgcgctcgaggcagagcgagagcgcctggccagcctgctcaccgcggaaaacggcaagccggtcgcacaagcccgaggcgaggtcggcgccgcaatttcagaggtccgctattacgccgggctggcgcggcacatcccgggtcacgtgctggagcccgagccaggcacgatatcgaccatcctgcgcgagccggccggcgtcgccgccatcatcgtcccctggaacgcgccggcggtgctgctcgtgcgctccctcgcgccagcgcttgccgcgggctgcacggcagtggtcaaatcggcagcgcaaaccacgctgttcacagccgcaatgctgcgcttgttcgagcgcacggccctgccggccggcgccgtcaatctggtctgcgaaacgggctatgcggcagcggaccacctggtgcgttcgcgcgacgtggacgtagtgagcttcacaggatcgaccgcaaccggcaagaagatcatgatcgccgctgcggacagcgtgaaaaaactctcgctggaactcggcgggaaatcgtgctgcctggtgttcgacgacgtcgatgcgcaagcggtcgcgaaacggcttgcgcttgccgccaccgtcatctcgggccagcaatgcaccgccgcgcggcgagtactggttcacgaagccatcgcgccacagatgcgccggcacctgaccgaggccctcgccgcgctgcgcctcgggcccggcatcgagcccgacacccaaatcggcccgctgatcgaccacccgacgcgcgcgatggtgagcgcgcaagtcgagcgcgcctgcgacgaggcggacacggtcctgctgcgcggcacgatgccgggcggcgcgctagcgcgcggcgccttcctcagccccacactagtggaacacagcgaccccggtgccttcttctgccaggaggagatcttcgggcccttcgtcacattcgagaccttcgcgaccgaagacgaggcgctagccaaggccaacaacaccgtcttcggcctgtccgccagcgtctggacgcaccacggcgagcgcgccatacgcctagcgcgggcgctgcgcaacggcacggtctgggtcaacgaccacaaccgcctgttcgccgaagcggagacgggcggctatcggcaaagcggccttggacggctccacggttatgacgccctcgcggacttcaccgagttgaagcacatctgcatccaggcgggcctgccgaaagggatgtcgcaggcgggctgcaggctcagtggggtagcagcgcgcgagcggatgggagtttccgtctag

SEQ ID NO: 17 - protein, 320 aa, Cupriavidus basilensis
  misc_feature: LysR-type transcriptional regulator, HMF 14
MGQLGHMDLKQIQYFIALFEDGSVTRAAKRLHIVQPALSMQIARLEEELNQKLFERGAHGMSPTAAGRQMYRLFLPIMRDITHAREQLVQRDEVVSGNITIGLIASIAEGALAEALTSFRMRYPQVEVTVADGYSTTLIDWVAGGQLDVAIINKPRAQLSLDSRPLLDEEMVLATSAAHGLDLPGSVQLASLPAVELVLPTKRHGLRGVLDSAAHQLDMMLTPKYEIDALGTIVKLVASTNMATILPRIAVQRAVDRGTLRVHAILAPRLIRHIVRISHPRRPLSTAAEALVSIIASEIQRTSDAATPAGGQDLNKEKME

SEQ ID NO: 18 - DNA, 963 bp, Cupriavidus basilensis
  misc_feature: LysR-type transcriptional regulator, HMF 14
atggggcaactcgggcatatggatttaaagcagatccagtacttcatcgcgctcttcgaggacggatcagtcactcgggccgccaagcggctgcatatcgtgcagcccgcgctgagcatgcagatcgccaggctggaagaagagctgaatcaaaaactgttcgagcgcggcgcgcatggcatgtcgccgaccgccgcgggccgccagatgtatcggctgttcctgcccatcatgcgcgacatcacgcatgcgcgcgaacaactggtgcaacgggacgaggtcgtctccggaaacatcacgatcggactgatcgcctccatcgccgagggtgctctcgccgaagcactgaccagctttcgtatgcgctatccccaggtcgaggtgaccgtggccgacggctacagcaccacgctgatcgactgggtagcgggcggccagctggacgtggccattatcaacaagccgcgcgcccagttgtcactcgactcccggccgctgctcgacgaagaaatggtgctggcaacgagcgcggcccacggcctggacctgcccggctcggtccagctagccagcctccccgcggtggaactggtgctgcccaccaaacgccacggcctgcgcggcgtgctcgacagcgcggcgcaccagttggacatgatgctcacgcccaagtacgagatcgacgcgctcggcaccatcgtcaagcttgttgcatcaaccaacatggccacgatcctgccgcgcatcgcggtgcagcgcgcggtggaccggggcacgttgcgcgtgcacgccatcctggcaccacggctaatccggcacatcgtgcgcatcagccacccccgccgccccttgagcacagcggccgaggcgctggtcagcatcattgccagcgagatccagcgtacgtcggacgcggcaacgcccgccggcggccaagacttaaacaaggagaagatggaatga

SEQ ID NO: 19 - protein, 497 aa, Cupriavidus basilensis
  misc_feature: 5-hydroxymethyl-2-furoic acid decarboxylase I, HMF 14
MSRQPAEKTNSASQAAPLPEGPLTLRSWLRHLGNTDRLAAIDEPVALEHTLAAVAKRLDGERAVLFRRPGGHAVPVVSGFMSRRAWIAEAMGVPEAGLLERMRSAAAQPLPVSEVAQGEAACQQVIHLDKVDLHKLLPIPTHSEHDNGPYITAGLAIARNPRTGVQNVSIHRIQVHAADRMAILLLPRHLDAFYRAAEECGEALPIAIVIGVDPLTMLASQAITPIDYDELEIAGALHGAPLEVIKCRTSDVRVPANAEIVIEGRLLPGEREMEGPFGEFPKYYSSAEPREVIQVDAVTHRHRPIYHTIVPAEMEHLLLGAIPREATLLAHLQRSHPGVQDVHLSVGGVCRYHLYVKLDKKREGEAKNVILSAFGAHYDIKQVVVVDTDVDVHDPAEVEWAVATRFQADQDLVVIAGAQGSVLDPSTTVAANLAGIDNPEPHLQGICAKMGLDATRPVKYAAHVFTRVRIPGESTIDLQALVSVDPSQWEAYLGEGA

SEQ ID NO: 20 - DNA, 1494 bp, Cupriavidus basilensis
  misc_feature: 5-hydroxymethyl-2-furoic acid decarboxylase I, HMF 14
atgtccaggcaacccgccgaaaagacgaacagcgcgtcgcaagccgcgccattgcccgaagggccgctgaccctgcgcagctggctgcgccacctcggcaacacggaccggctggctgcaatcgacgagccggtggcactagagcacacccttgcagccgtcgccaagcggctcgatggcgagcgcgcggtgctcttccgccggcctggcggccatgccgttccggttgtgagcgggttcatgtcgcgccgcgcgtggatcgccgaggcgatgggcgtgcccgaggccggcctgctcgagcgcatgcgcagcgccgccgcacagcccttgccggtgagcgaggtggcccagggcgaggccgcatgccagcaggtcatccacctggacaaggtggacctacacaagctgttgcccatcccgacccacagcgagcatgacaacggcccctatatcactgcaggcctggccatcgcgcgcaacccgcgcaccggcgtgcagaacgtatcgatccaccgcatccaggtgcatgccgcggatcgcatggccatcctgctgctgccgcggcatctcgatgcgttctaccgcgcggcggaggaatgcggcgaggcgctgccgattgccattgtcatcggtgtcgatccactcaccatgcttgcttcgcaggccatcacgcctatcgactatgacgagttggagatcgccggggcattgcacggcgcgccgcttgaagtgatcaagtgccgcaccagcgatgtgcgtgtgccggccaatgccgagatcgtgatcgagggccggcttctgcccggcgagcgcgagatggaagggccctttggcgagtttcccaagtactacagcagcgccgagccgcgcgaggtcatccaggtcgacgccgtcacgcaccgtcatcggccgatctatcacaccatcgtgccggcggagatggagcacctgctgctcggggcgattccgcgcgaagcgaccttgctggcgcatctgcagcgcagccatcccggggtgcaggatgtgcatctgtcggtgggcggcgtatgccggtaccacctgtatgtaaagctcgacaagaagcgcgagggcgaagcgaagaacgtcattctctcggcgtttggcgcgcactacgacatcaagcaggtggtcgtggtggataccgatgtcgacgtccacgacccggccgaggtggaatgggccgtcgcgacccgcttccaggcagaccaggacctggtggtgatcgccggggcgcagggctcggtgctcgacccctccacgaccgtcgccgccaacctcgccggcatcgacaatcccgagccgcacctgcaaggcatctgcgccaagatggggctggacgcgacccgcccggtcaagtacgcggcgcatgtgttcacgcgcgtgcggattcccggcgagtcaaccatcgatttgcaggcgctggtgtcggtcgacccatcgcaatgggaagcgtatctcggcgaaggagcttga

SEQ ID NO: 21 - protein, 234 aa, Cupriavidus basilensis
  misc_feature: 5-hydroxymethyl-2-furoic acid decarboxylase II, HMF 14
MCSRACGFPASQPSICRRWCRSTHRNGKRISAKELDVMASQRRIIVGISGASGAAIGVNLLKAMRGLDGVESHLIVSASGMLTATQELGIKRSELEALADVVHNVRDIGAAVASGSFVTEGMVVAPCSMKTLASVANGFSDNLLTRAADVVLKERRRLVLVARETPLNLAHLRNMLHATEMGAIVMPPVPAFYSHPTSIEDVVNHTVGRILDLFQIEHGTLVSRWSGLAHDFAR

SEQ ID NO: 22 - DNA, 705 bp, Cupriavidus basilensis
  misc_feature: 5-hydroxymenthyl-2-furoic acid decarboxylase II, HMF 14
atgtgttcacgcgcgtgcggattcccggcgagtcaaccatcgatttgcaggcgctggtgtcggtcgacccatcgcaatgggaagcgtatctcggcgaaggagcttgatgtcatggccagccagagacgcatcattgtcggtatatccggcgccagcggcgcggccatcggcgtcaatctcctcaaggcgatgcgcggcctggacggcgtcgagtcgcacctcatcgtgtcggcctccggcatgctcaccgcgacccaggaactcggcatcaagcgcagcgaactcgaggcgcttgccgacgtagtgcataacgtgcgcgatattggcgcagcggtggccagcggctctttcgtgaccgagggcatggtggtcgcgccgtgctcgatgaagacactagcgtcggtggccaacgggttttccgacaacctgctgacccgcgccgcggacgtggtgctcaaggagcggcggcgcctggtgctggtggcgcgcgaaaccccgctgaacctcgcgcacctgcgcaacatgctgcacgccaccgagatgggcgcgatcgtgatgccgcccgtgcccgccttctactcgcatccgaccagcatcgaggatgtggtcaatcacaccgtgggccgcatcctggacctgttccagatcgagcacggcacgctggtcagccgctggtcgggcctcgctcacgactttgcccgctga

SEQ ID NO: 23 - protein, 328 aa, Cupriavidus basilensis
  misc_feature: Extracytoplasmic solute receptor; probable orphan gene, HMF 14
MLNRQETDMKTWLAHAGLALLLLTGLAHAQGYPTKPIRIVVPYPPGGFNDTLARIVGSRLTAAWGQPVVVDNKPGAGTIIGTSFVAKAAPDGYTLLVVQFPFGANPWLYKSLPYDTLKDFTPVILAGESPMTLVVTNGSPIRSVDDLVKSAKGTPGKINYGSSGSGSSNHLAMALFERSAGITLAQVPYKGSTPMLTDLAGGQVEVAFDALPHVLPFVRSGKVRALAVADRSRFASLSTVPTMAESGLPGYDASSWHGIVAPAGTPPEIVRKLNAQINDALRTADVRNLFHEQGVRPDGGSPADFSAFIGRELAKWKQVVHDAAIPLQ

SEQ ID NO: 24 - DNA, 987 bp, Cupriavidus basilensis
  misc_feature: Extracytoplasmic solute receptor; probable orphan gene, HMF 14
atgctgaacaggcaggagacagacatgaaaacttggctcgcccatgccgggctcgctttgctgttgctcacggggcttgcccatgcgcaagggtatcccaccaagccgatccgcatcgtggtgccgtacccgcccggcggcttcaatgacacgctggcccgcatcgtcggcagcaggctcaccgcagcctggggccagcccgtggtggtggacaacaagcccggtgcgggcaccatcatcggcacctcgttcgtggccaaggccgcgcccgatggctacacgctgctggtggtgcagttccccttcggtgccaatccctggctctataagtcgctgccgtacgacaccctcaaggacttcacgcccgtcatcctggcaggcgagtcgccgatgacccttgtggtgaccaacggatcgccgatacgctccgtggacgatcttgtcaaatccgctaagggcacgcccggcaagatcaactacggctcgtcgggcagcggctcgtccaaccaccttgccatggcgctgttcgagcgcagtgccggcatcacgctcgcccaggttccctacaagggcagcacgcccatgctgaccgatctggccggcggccaggtcgaagtggccttcgacgcgttgccccatgtgttgcctttcgtgaggtccggcaaggtgcgcgcacttgccgtcgccgaccggagccgctttgcgtcgctttccacggtgcccaccatggccgagagcggcctgcccggctacgacgcgtcatcctggcacggcatcgtcgcgccggccggcacgcccccggagatcgtgcgaaagctgaacgcgcagatcaacgacgcgctgcgcacggcggatgtgcgcaatctcttccacgagcagggcgtgcgtcccgacggcggcagccctgccgacttctctgcgtttatcggcagggaactggcgaagtggaagcaggtggtgcatgacgcggccatccccttgcaatga

SEQ ID NO: 25 - protein, 579 aa, Cupriavidus basilensis
  misc_feature: HMF/furfural oxidoreductase, Small orf, HMF14
MDTPRERFDYVIVGGGSAGCVLANRLSQDPAIRVALIEAGVDTPPDAVPAEILDSYPMPLFFGDRYIWPSLQARAVAGGRSKVYEQGRVMGGGSSINVQAANRGLPRDYDEWAASGASGWSWQDVLPYFRHLERDVDYGNSPLHGSHGPVPIRRILPQAWPPFCTEFAHAMGRSGLSALADQNAEFGDGWFPAAFSNLDDKRVSTAIAYLDADTRRRANLRIYAETTVRKLVVSGREARGVIAMRADGSRLALDAGEVIVSAGALQSPAILMRAGIGDAGALQALGIEVVADRPGVGRNLQDHPALTFCQFLAPQYRMPLSRRRASMTAARFSSGVPGGEASDMYLSSSTRAGWHALGNRLGLFFLWCNRPFSRGQVSLAGAQPDVPPMVELNLLDDERDLRRMVAGVRKLVQIVGASALHQHPGDFFPATFSPRVKALSRVSRGNVLLTELLGAVLDVSGPLRRSLIARFVTGGANLASLLTDESALEGFVRQSVFGVWHASGTCRMGAHADRSAVTDAAGRVHDVGRLRVIDASLMPRLPTANTNIPTIMLAEKIADTMQAERRAVRPASSEVAHPS

SEQ ID NO: 26 - DNA, 1740 bp, Cupriavidus basilensis
  misc_feature: HMF/furfural oxidoreductase, Small orf, HMF 14
atggatacgccgagggagcgtttcgactacgtgattgttggcggcgggtccgccggttgcgtactggccaatcgcctgtcgcaggacccggccatccgcgtcgcgctgatcgaggcgggcgtcgatacgccgccggacgctgtgccggcggagatcctcgacagctatccgatgcccttgttcttcggtgaccggtatatctggccatcgctgcaagcccgcgccgtggcagggggcaggtccaaggtctacgagcaagggcgcgtcatgggcggcggctccagcatcaacgtgcaggcggcaaaccgcgggctgccgcgcgactacgatgagtgggccgcgtcgggcgcgtccggatggtcgtggcaggatgtgctgccgtatttccgccaccttgagcgcgatgtggattacggcaacagcccgctgcacggcagccacggaccggtgccgatccgccgcatcctgccgcaggcttggccgccgttctgcacggagtttgcgcacgcgatgggccgcagcggcttgtccgcgctggccgaccagaacgcggagttcggcgatggctggtttccggccgccttctcgaacctggatgacaagcgggtttcgaccgccatcgcctatctcgacgcggatacgcgccggcgggccaatctgcggatctatgccgagacaacggtgcgcaagctcgtcgtatccggccgggaagcgcgtggggtgatcgccatgcgggccgatgggtcgcggctggcgctggacgccggggaggtcatcgtgtccgcgggcgccttgcagtcgcccgccatcctgatgcgcgcggggatcggcgacgccggcgcgctgcaggccctcggcatcgaggtcgtagccgaccgacccggcgttggccgcaatctccaggatcatcccgcgctgacgttctgccagttcctcgcgccccagtaccgcatgccgctctcgcgccggcgcgctagcatgacggcggcgcggttctcatcgggggtgccaggtggcgaggcgtcggacatgtacctgtccagttccacacgggcaggctggcatgcactcggtaatcggctcggcctcttcttcctgtggtgcaatcggccattctcgcgcgggcaggtgagccttgcgggagcccagccggatgtgccgcccatggtggagctcaacctgctcgacgacgagcgggatctgcggcgcatggtggccggcgtacgcaagttggtgcagatcgtgggtgcgtcggccttgcatcagcatcccggtgatttcttccccgctacgttttcgccgcgcgtcaaggcgctgagccgcgtgagccgcggcaatgtgttgctcacggagttgctgggggcagtgcttgatgtctcggggccgctgcgcagaagcctgatcgcgcgctttgtcacgggcggcgcaaacctggccagcctgctgacggatgagtccgcgctagagggcttcgtgcgccagagcgtcttcggggtctggcatgccagcggcacttgccggatgggcgcgcatgcggaccggagcgcggtgacggatgcggcgggccgcgttcacgatgttggcaggctgcgcgttattgacgcctctctgatgccgcggctgccgacggccaataccaacatccccaccatcatgctcgcggaaaagattgccgacaccatgcaagccgagcgccgcgcggtccggccggcatcgagcgaagttgcccatccgagttga

SEQ ID NO: 27 - protein, 231 aa, Cupriavidus basilensis
  misc_feature: Fatty acid hydroxylase, HMF 14
MKYDDEIRARSYRFRDEYVAATPAWYRGELHLAFTLLFTGGVIAWCAMKLQAPTLAQWLAIVPIFLLGNWAEWAAHRYILHRPTRLFSAIYKRHCAVHHRFFTHLTLSTKARSTGAPCCFHPLRGSLRAGAVRRAVIGVAFSKNAGYIALMTMAAYYLMYEGLHTLSHITDSPLLDRMPFVGTVRRLHVTHHDPELMATQNFNLTFPICDTLFGTRSDVPREVRSPMQGQG

SEQ ID NO: 28 - DNA, 696 bp, Cupriavidus basilensis
  misc_feature: Fatty acid hydroxylase, HMF 14
atgaaatacgatgacgaaatccgagcacgctcttaccgcttccgcgacgaatatgtcgccgccacccctgcgtggtatcgcggcgaattgcatctggccttcacgctgctattcaccggcggggtgattgcctggtgcgcgatgaaactgcaagcaccgacgctggcgcagtggctcgcgatcgtgccaatcttcttgctcgggaactgggccgagtgggccgcgcaccgctatatattgcatcgaccgacgcgcttgttcagtgcgatctataaacggcattgcgctgtgcatcatcgcttcttcacacatctgacgctgagtacaaaggccagaagcactggcgcgccttgttgtttccaccctttgcgcggtagccttcgtgctggcgctgtgcgtcgcgctgtgatcggcgtggcgttctcgaagaacgcgggctatatcgcgctgatgacgatggcggcgtactacctgatgtacgagggcctgcatacgctgtcgcacatcacggatagcccgctgctggatcggatgccgttcgtggggaccgtgcggcgcctgcacgtcacgcatcacgatcctgagctgatggccacccagaacttcaacctgaccttcccgatctgcgacacgctgttcggcacgcgcagcgatgtgccgcgcgaggtgcgaagcccgatgcaagggcaggggtag

SEQ ID NO: 29 - protein, 74 aa, Cupriavidus basilensis
  misc_feature: Truncated LysR-type transcriptional regulator, HMF 14
VQFVEASRFATVLPRIVVQRAVDQGRLSMYPILAPRIVRHLVCVSHPQRPLSTATVAPIAIVAAEIRRVSGTAG

SEQ ID NO: 30 - DNA, 225 bp, Cupriavidus basilensis
  misc_feature: Truncated LysR-type transcriptional regulator, HMF 14
tcagccggcggtgcctgacaccctgcggatctccgcggccacgatcgcgatcggcgcaacggttgccgtgctgagcggacgctgcggatggctcacgcacaccaggtggcgcacgatgcgcggcgccaggatggggtacatgctcagccggccctggtccaccgcgcgttgcaccacgatgcgcggcagcaccgtggcaaaacgcgaggcctcgacgaactggac

SEQ ID NO: 31 - protein, 441 aa, Cupriavidus basilensis
  misc_feature: Major facilitator superfamily transporter, HMF 14
MEAVAKKRTETISEALPAATNRQVFGAVTASCMGWALDLFDLFILLFVAPVIGRLFFPSEHAMLSLAAVYASFAVTLLMRPLGSAIFGTYADRHGRKGAMVVAVTGVGLSTAAFGLLPTVGQVGLLAPALFILLRLVQGIFVGGVVASTHTIGTESVPPSWRGAVSGLVGGGGAGIGALLASITYMAMTALFPGEAFDAWGWRCMFFSGIISSVLGLFIFNSLEESPLWKQLQAAKGHAAPVENPLRVIFSRQYRGVLFVNILLTVGGGSAYYLTSGYLPTFLKVVVKAPAGASAAILMASSVGVIVASIIAGHLSTLIGRKRAFLLIGALNVVLLPLIYQRMPAAPDVTTLGIYAVALAMLGSTGFAPILIFLNERVSHQHPCYGNWPVMEYRLCHRRHDATFASLCAAPPRLPKCWGSRRGVKAFTAGAAIVWNAPLGE

SEQ ID NO: 32 - DNA, 1326 bp, Cupriavidus basilensis
  misc_feature: Major facilitator superfamily transporter
atggaagccgtagcaaagaagcgtacagagacgatcagcgaggcgctgccagcggcgaccaatcgccaggtgtttggtgccgtgacggcgtcgtgcatgggatgggcgctggacctgttcgacctgttcatcctgctgttcgtggcgcccgtgatcggcaggctgtttttcccgtcggagcacgccatgctgtcgctggcggcggtgtatgcgtcgtttgccgtgacgctgctgatgcggccgctcggctcggcgatcttcggcacttatgccgaccgccacggccgcaagggggcgatggtagttgccgtcactggcgttggcttgtccacggcggcgttcggcctgctgcctacggtgggtcaggtggggctgcttgcgccagccttgtttatcctgctgcggctggtgcagggcatcttcgtaggtggcgtggtggcatccacccacaccatcggtaccgaatcggtgcccccgtcctggcgcggcgccgtctccgggctggtcggtggcggtggcgcgggcatcggggcactgctggcttccattacctacatggcgatgaccgcgctgtttccgggggaagcgttcgatgcctggggttggcgctgcatgttcttctccggcatcatcagctcggtgctcggcctgttcatcttcaactcgctggaggagtctccgctgtggaagcagttgcaggcggccaaggggcacgccgcgccggttgagaacccgctgcgcgtgatcttctcccgccagtaccgtggcgtcctcttcgtcaacatcctgctcaccgtgggcggtggcagcgcctactacctgacctccggctatctgccgaccttcctcaaggtggtggtgaaggcaccggctggcgcatccgcagccatcctgatggccagcagtgttggcgttatcgtggcatcgataattgccggtcacctcagcacgctgattggtcgcaagcgagccttcctgctgatcggcgccttgaacgtggtgctgctgccgttgatctaccaacggatgcccgcggcgccggatgtcaccacgcttggcatttatgccgtggcgctggcgatgctgggcagcaccggcttcgccccgatcctcattttcctgaacgaacgggtttcccaccagcatccgtgctacgggaactggcctgtcatggaatatcggctttgccatcggcggcatgatgcgacgtttgcgtcgctgtgcgcagcacccccgcgactgccaaagtgctggggatctcgtcgcggtgtcaaagcatttactgccggtgcggcgatcgtctggaacgcgccgctgggggagtga

SEQ ID NO: 33 - protein, 314 aa, Cupriavidus basilensis
  misc_feature: LysR-type regulator, HMF 14
MDIKQIQYFIALFEDGSVTRAAKRLNIVQPALSMQIAKLEEEFGQKLFDRIPHGMVPTAAGRMMYRLCLPITRDLANARQQLMQREEQVAGNISIGMVASETESVLVGSLVRFNARYPNVEVSVADGFSATLIDLVSAGQLDAAVVHKPRGKLSLHVQSLHDEEMVLVTSAEHGPGTAGGGGTHQAARAGTGAAHQAPRLARRAGRGHATGRRDPAAEVRDRHPRHHRPVRRGHAFCHGAAAHRGATRGGRGPAAHVPHPGAAHRAPPGVREPSAAPAQHGSRCADRDRGRGDPQGVRHRRLIRVPGIRKGTIR

SEQ ID NO: 34 - DNA, 945 bp, Cupriavidus basilensis
  misc_feature: LysR-type regulator,  HMF 14
atggacatcaaacagatccagtacttcattgcgctgttcgaagatggctccgtcacgcgcgcggccaagcggctgaatatcgtgcagccggcgctcagcatgcagatcgccaagctggaagaggagttcggacagaagctgttcgatcgcatcccgcacgggatggtgccgacagcggccggccgcatgatgtaccggctgtgcctgccgatcacgcgggacctcgccaacgcccggcagcaactgatgcaacgcgaggagcaggtggccggcaacatctccatcggcatggtggcgtcggaaacggaaagcgtgctggttggctcgctggtgcgcttcaacgcgcgctacccgaatgtggaggtatcggtggcggacggattcagcgcgacgctgatcgacctggtctccgccggccagctcgacgcggccgtggtgcataagccccgtggcaagctctcgctccatgtgcaatccctgcatgacgaggaaatggtgctggtcaccagcgccgagcatgggcccggaactgccggcggcggtggaactcaccaagctgcccgggctggaactggtgctgcccaccaagcgccacggcttgcgcggcgtgctggacgcggccacgcaactggaagacgtgaccctgcagccgaagttcgagatcgacatcctcggcaccatcgtccagttcgtcgaggccacgcgttttgccacggtgctgccgcgcatcgtggtgcaacgcgcggtggacgagggccggctgcgcatgtaccccatcctggcgccgcgcatcgtgcgccacctggtgtgcgtgagccatccgcagcgcccgctcagcacggcagccgatgcgctgatcgcgatcgtggccgaggagatccgcagggtgtcaggcaccgccggctgatccgggtgcccggcatcaggaagggcacgatacggtag

SEQ ID NO: 35 - protein, 1015 aa, Cupriavidus basilensis
  misc_feature: Furoyl-CoA dehydrogenase large subunit, HMF 14
MSDKRSLPAAAAAIHLESAAAGAASRQRHVGRSMERLEDAAILTGRGRYGDDLGVKPGTLHAAIVRSPHAHAELGTIDATAALAAPGVHAVLTGADLAAWSRPFVVAVKSPMEQWALAMDRVRYVGEPVAVVMAESRALAEDALDLVRVNYRVLPPVVSIEAALADDAPILHPGVGANVVSDRHFRYGEPEAAFAAAPHRVTLTAHYPRNTCTPIECGVVIAEFLPGDEGYDVTSNFMGPFSLHAVMAMALKVPANRLRHKAPRDSGGSFGVKQAVFPYAVLMCLASRKAGAPVKWVEDRLEHLSAATSATARLSTLEAAVESDGRIKALTYDQIEDCGGYLRAPEPATFYRMHGCLTGAYDIPNLLVRNRVVMTNKTPTGLVRGFGGPQVYFALERLVHRIATQLGLDPLDVYRRNFVAADAFPYRAAAGALLDSGNYQLALARALEEGGYYELTCRRDVARAEGRLYGIGFAAIVEPSVSNMGYITTAMPAEARKKAGPKNGAIASATVSVDLLGGVVVTIASTPAGQGHMTVCAQVVADVLGVNPADVVVNVEFDTHKDAWSVAAGNYSSRFAGAVAGTVHLAAERVRDKLARIVAPQFGCTPAEVVFEDGRIARKGAPESGLPFTRVASNAPHWSPQQLPAGEEPGLRETVFWSPPNLEAPDENDRINTSAAYGFAFDMCGVEIDRATGRVRIDRYVTAHDAGTLLNPALADGQIRGAFAQGLGAALMEEFRYGADGSFQSGTLADYLMPTTCEVPDPVIVHLETPSPFTPLGAKGLGEGNNMSTPPCIANAVADALGEQDIRLPLTPAKVMAMVGFDDPPPSRPELLEAMREAAVPAARKGSAKALTARGSVDLDATPEAIFAVLMDPQALARVVPGCHALERTAENHYRADVTVGVGMIKARFEAEIALSDLDPPRRLRLAGAGMSSLGSARGAGLVELVPHGSGTRLSYDYEAEVSGKVAAVGGRMLEGAAKVVLRQLFESLGRQAAGKPVRPQGWLARLLARLGVRR

SEQ ID NO: 36 - DNA, 3048 bp, Cupriavidus basilensis
  misc_feature: Furoyl-CoA dehydrogenase large subunit, HMF 14
atgagcgacaagcgcagcctgcccgccgcagcggcggccatccaccttgaatccgcagccgccggcgcggcctcgcgccagcgtcacgtcggccgctcgatggagcggctcgaggacgccgcgatcctcaccggccgtggccgctatggcgacgacctgggcgtgaagccgggcaccctgcacgcggccatcgtgcgttccccgcatgcacatgccgaacttggcaccatcgatgccaccgccgcgcttgccgcgccgggcgtgcatgccgtgctgaccggcgccgacctggcagcctggtcgcgtcccttcgtggtcgccgtgaagtcgccaatggagcaatgggcactggccatggaccgcgtgcgctatgtgggcgagccggtggccgtggtaatggccgaaagccgtgccctggccgaagacgcgctcgacctggtgcgggtgaattaccgcgtgttgccgccggtggtatcgatcgaggctgcgctggccgacgatgcgcccatcctgcatcccggcgtaggcgccaatgtggtgagcgaccgccacttccgctacggcgagccagaggccgcctttgccgccgcgccgcaccgggtcacgctgaccgcgcactatccgcgcaacacctgcacgccgatcgaatgcggcgtggtgattgccgagttcctgcccggtgacgaaggctacgacgtcacctccaatttcatggggccgttctcgctgcatgcggtgatggcgatggcgctcaaggtgccggccaaccggctgcgccacaaggccccgcgcgattccggcggcagctttggcgtgaagcaggcggtgtttccttacgcggtgctgatgtgcctggcgtcccgcaaggccggcgcgcctgtaaagtgggtggaggaccgactcgaacatctcagcgcggccacctccgccaccgcgcggctgtccacgctggaagccgcggtggaatcagacggccgcatcaaagcgctgacctacgaccagatcgaagactgcggcggctatctgcgcgcgccggagcccgccactttctaccgcatgcacggctgcctgactggcgcctacgacatccccaacctgctggtgcgcaaccgcgtggtgatgaccaacaagacgcccaccggcctggtgcggggctttggcggcccgcaggtgtattttgcgctggagcggctggtgcatcgcatcgcgacacaactcgggctcgatccactcgatgtgtatcgccgcaactttgtcgccgccgatgcctttccctatcgcgccgcggcgggcgcgttgctggactccggcaactaccagctggcgctggcgcgcgcgctggaagaaggcggctactacgagctgacgtgccgccgcgatgtcgcacgcgccgagggccggctctatggcatcggctttgccgccatcgtcgagccgtcggtgtcgaacatgggctatatcaccaccgccatgcccgccgaggcgcgcaagaaggcggggccgaagaacggcgccatcgccagcgccacggtcagcgtcgacctgctcggcggcgtggtggtcaccattgcctcgacgccggccggacagggccatatgacagtgtgcgcgcaggtggtggccgatgtgctcggcgtgaatccggccgacgtggtcgtaaacgtcgagttcgatacgcacaaggatgcatggtcggtcgccgccggcaactactccagccgtttcgccggcgccgtggccggcaccgtgcacctggccgccgagcgggtgcgcgacaagctggcgcgcatcgtggcgccgcagttcggctgcacgcccgccgaggtggtgttcgaagacggccgcattgcccgcaaaggcgcgcccgaatccggcctgcccttcacgcgcgtggccagcaatgcgccgcactggtcaccgcagcagttgccggcgggagaagagcctggcctgcgcgagacggtgttctggtcgccgcccaacctggaagcgccggatgagaacgaccgcatcaacacctcggccgcctacggctttgccttcgatatgtgcggcgtggagatcgaccgcgccacggggcgcgtgcgcatcgaccgctacgtgaccgcgcacgacgccggcacgctgctcaacccggcgctggccgatggccagatccgcggcgccttcgcccaagggctgggcgcggcgctgatggaggagttccgctatggcgccgacggcagcttccagtccggcacgctggccgactacctgatgccgaccacctgcgaggtgccggacccggtgatcgtgcacctggagacgcccagccccttcacgccgctcggcgccaagggcctgggcgagggcaacaacatgagcacgccgccctgcatcgccaacgcggtagccgacgcgctcggcgagcaggatatccgcctgccgctgaccccggccaaggtgatggccatggtgggcttcgacgatccgccgccgtcgcgccccgagctgctcgaagcgatgcgcgaggccgccgtgcccgcggcccgcaagggcagcgcaaaggcgctcaccgcgcgcggctcggtggatctggatgccacgcccgaagccatcttcgccgtgctgatggacccgcaggccctggccagggtagtgccaggctgccacgcgctggagcgcaccgccgagaaccactaccgcgccgatgtgacggtgggggttggcatgatcaaggcccgcttcgaggcggagatcgcactgtcagacctcgatcccccgcgccggctgcggctggcaggcgcaggcatgtcctcgctgggcagtgcacgcggcgccgggctggtggagttggtcccgcatggcagcggcacgcgcctgagttacgactacgaggccgaggtatccggcaaggtcgctgccgtgggcggccgcatgctggagggcgccgccaaggtggtgctgcgccagttgtttgaatcgctcggccgccaggccgccggcaagccggtacggccgcaaggctggcttgcccgactgctggcccgcttgggagtacgccgatga

SEQ ID NO: 37 - protein, 271 aa, Cupriavidus basilensis
  misc_feature: Furoyl-CoA dehydrogenase FAD binding subunit, HMF 14
MKPSAFDYLRAETTQHALEALARGGEGARVLAGGQSLMAVLNMRLAQPQLLIDISRTAELDTVRVEDAHLVVGAAATQGSVEWRRSLADEVPLLAMAFPHISHFQIRNRGTVCGSVAHADPSAELPLVLTALGGEVVLRSARRRRVLPAASFFQGMLMTAREPDELVEAVRFPLRRPGARYGFAEFSARHGDFALVACAATVTDDAIALAVGGVADRPVLETWPRLQGKDLEQAINDFSWKLGAQDDAHISAQYRRHLVRQLSMRVIEEAK

SEQ ID NO: 38 - DNA, 816 bp, Cupriavidus basilensis
  misc_feature: Furoyl-CoA dehydrogenase FAD binding subunit, HMF 14
atgaaaccgtctgctttcgattacctgcgcgccgagaccacgcagcacgcgctcgaggcgctggcccgtggcggcgagggcgcgcgcgtgctggccggcggccagtcgctgatggcggtgctcaatatgcgcctggcgcagccgcaactgctgatcgatatctcgcgcaccgccgagctggacacggtgcgggtggaagacgctcacctcgtggtgggtgccgcggccacgcagggcagcgtcgaatggcgccgctcgctggccgacgaggtgccgctgctggccatggcctttccgcatatctcgcatttccagatccggaatcgcggcaccgtgtgcggctcggtcgcccatgccgacccgagcgcggaattgcccctggtgctgaccgcgctgggcggcgaggtggtgctgcgttcagcccgccgccgccgcgtgctgcctgcggccagcttcttccagggcatgttgatgacggcgcgcgagcccgacgagctggtggaggccgtgcgctttccgctgcggcgccccggggcgcgctacggctttgccgaattctccgcgcgccacggcgattttgcgctggtggcctgcgcggccaccgtgaccgatgacgccatcgcgctggcggttggcggcgtggcggacaggccggtgctggaaacctggccgcgcctgcagggcaaggacctggagcaggccatcaacgatttcagttggaaactgggcgcgcaggacgacgcccatatcagcgcgcagtaccgccggcacctggtgcggcaactgagcatgcgtgtgatcgaggaggcaaaatga

SEQ ID NO: 39 - protein, 192 aa, Cupriavidus basilensis
  misc_feature: Furoyl-CoA dehydrogenase 2Fe-2S iron sulfur subunit, HMF 14
MSKTNKGAVSGKPAEVMQRQEQRRITLTLNGRERSGHCEPRELLSDFLRHELGATGTHVGCEHGVCGACTVRVDGVAARSCLMLAVQAEHRAIDTVEGLAPAEGLGDLQEAFRRHHALQCGFCTAGILMSCADYLERVPEPSEAQVRDMLSGHLCRCTGYTPIVAAVLDVAAIRARARHAAAGVDTQEARNA

SEQ ID NO: 40 - DNA, 579 bp, Cupriavidus basilensis
  misc_feature: Furoyl-CoA dehydrogenase 2Fe-2S iron sulfur subunit, HMF 14
atgagcaagaccaacaagggcgccgtgtccggcaagcccgccgaggtaatgcagcgccaggagcaacgccgcatcaccctgacgctgaacggccgcgagcgcagcggccattgcgagccgcgcgagctgctgtcggacttcctgcgccacgagctcggcgccaccggcacccatgtgggttgcgagcacggcgtctgcggcgcatgcacggtacgcgttgacggcgttgccgcgcgctcgtgcctgatgctggcggtgcaggccgagcaccgggccatcgataccgtcgaagggctggcgccggccgagggactgggcgacctgcaagaagccttccgccgccaccacgcgctgcagtgcggcttctgcaccgcaggcatcctgatgtcgtgcgcggactacctggagcgcgtgccggaacccagcgaggcgcaggtgcgcgacatgctgtccggccacctgtgccgctgtacgggctacacccccattgtggccgccgtgctcgacgtagccgcgatccgtgcacgggctcgtcatgctgctgccggcgtagatacccaggaggcccgcaatgcttga

SEQ ID NO: 41 - protein, 532 aa, Cupriavidus basilensis
  misc_feature: Furoyl-CoA syntethase, HMF 14
MLDLGRTFLQSVERSPHTPAIVDGDLMLTYAQWYERIRCVASGLREIGLAPGDRLLAVLQNRWEMATLYWACQFAGIVMVPLNWRAKPEELDYCVQDAGVKALVFEPVSADAVLGSPAAQAVPCIALDCAAGGSMSFASLLDSVALHGGPVAEAGDVSLMLYTSGTTGKPKGVPRRHQHERAAALAHVAQNLYRHGERTLGVMPLYHTMGVRSLLAMALVDGLFVCVRRWNAGQALEEINTHRISCLYLVPTLYHDLLADPGFDACIVRSVTKLGFAGASMNDGLLRRLALAFEPELFVNHYGSSEVYTFSVDQRATRKPGSAGRAGINTRLRVVRLDARSPDDLAATGEEGQIIADLRGDEAFEGYWNRDDANAKSLRDGWYFTGDTGYFDAEGDLFVSGRVDDMIISGGENISPVDIESVLSLHPAVDEVAVAGVPDPRWGQKVVAFVKPRGNIDAQALDTYCRGSDLVNFKRPRDYVFVEEIPKSPVGKILRRKLSAGEYALAPHSMSPDPNTNPNQAADAAPVDTIKE

SEQ ID NO: 42 - DNA, 1599 bp, Cupriavidus basilensis
  misc_feature: Furoyl-CoA syntethase, HMF 14
atgcttgatctaggccgcaccttcctgcaaagcgtggagcgcagcccgcacacgcccgccattgtcgacggcgacctgatgctcacctatgcgcaatggtacgagcgcatccggtgcgtggcgtccggcctgcgcgagatcggcctcgcgccgggcgatcgcctgctggccgtgttgcaaaaccgctgggaaatggccacgctgtactgggcctgccagttcgccggcatcgtgatggtgccgctgaactggcgcgccaagccggaggagctcgattactgtgtgcaggatgccggcgtcaaggcgctggtgttcgagccggtcagcgccgatgcggtgctgggcagccccgcggcgcaggccgtgccctgcattgcgctggactgcgcggctggcggctcgatgtccttcgcttcgctgctggacagcgtcgcgctgcatggcggcccggtggcggaagcgggcgatgtctcgctgatgctctacacctcgggcaccaccggcaagcccaagggcgtgccgcgccgccaccagcacgagcgcgccgcggcgctggcgcacgtggcgcagaacctgtatcgccatggtgagcgcacccttggcgtgatgccgctctaccacaccatgggcgtgcggtccctgctggccatggcgctggtggacggcctgttcgtctgcgtgcggcgctggaacgccgggcaggcgctcgaggagatcaacacccaccgaatcagctgcctgtacctggtgccgacgctgtaccacgacctgctggccgatccggggttcgatgcctgcatcgtgcgcagcgtgaccaagctcggctttgccggcgcctcgatgaacgacggcctgctgcgccgccttgcgctggccttcgagccagagctgttcgtgaaccactacggctcatccgaggtgtacaccttcagcgtggaccagcgcgccacccgcaagcccggcagcgccgggcgcgccggcatcaacacgcgcctgcgcgtggtgcgcctggatgcccgctcacccgacgatctggcggctaccggcgaggaaggccagatcattgccgacctgcgcggcgacgaggccttcgagggctactggaaccgcgacgacgccaacgccaaatcgctgcgcgatggctggtacttcaccggtgacaccggctacttcgatgccgagggcgatctcttcgtcagcggccgggtggacgacatgatcatcagcggcggcgagaacatctccccggtcgatatcgaatcggtgctgtcgctgcatccggcggtcgatgaggtggcggtggccggcgtgccggatccgcgctggggccagaaggtggtggctttcgtcaagccgcgcggcaacatcgacgcgcaagccctggatacctactgccgcggctcggacctggtcaatttcaagcgcccgcgcgactacgtcttcgtggaggagattcccaagtcgccggttggcaagatcctgcggcgcaagttgtccgccggcgaatacgcgctggccccgcattccatgagccccgaccctaataccaaccccaaccaggctgcggacgccgcgcctgtcgataccatcaaggagtaa

SEQ ID NO: 43 - protein, 271 aa, Cupriavidus basilensis
  misc_feature: 2-oxoglutaroyl-CoA hydrolase, HMF 14
MTQATEMIHPDQQRLQQLDGFSVEIDAGRERADIILHRPPYNVIAMAARDQLRAVIEALDADDRVRVIVLRSQGEHFSSGGDIKGFLEASPEHVSQLAWNVAAPARCSKPVIAANRGYCFGVGFELSLACDFRIATETTQYALPEQKLGQIPGSGGSARLQKMVGIGRTKDIVMRSRRISGKQAYEWGIAVECVADAELEAATDALVDELRGFSPLAQRTAKKLLNDTEDAPLSIAIELEGHCYSRLRSSDDFREGVEAFHGKRKPAFRGS

SEQ ID NO: 44 - DNA, 816 bp, Cupriavidus basilensis
  misc_feature: 2-oxoglutaroyl-CoA hydrolase, HMF 14
atgacccaggcaaccgagatgatccatcccgaccagcagcggctccagcaactcgacggcttctccgtggagatcgatgccgggcgcgagcgtgcggacatcatcctgcaccgtccaccctacaacgtgatcgccatggcggcgcgcgaccagttgcgtgccgtcattgaagcgctggatgccgacgatcgcgtgcgcgtgatcgtgcttcgttcgcaaggcgagcatttttccagcggcggcgatatcaagggcttcctggaggcatcgcccgagcatgtctcgcaactggcctggaacgtggcggcgccggcgcgctgcagcaagccggtgattgccgccaaccgcggctactgctttggcgtgggcttcgagctgtcgctggcgtgcgacttccgcatcgccaccgagaccacgcagtacgcgctgccggaacagaagctcggccagatccccggctcgggcggctcggcgcgcctgcagaagatggtgggcatcggccgcaccaaggacatcgtgatgcgctcgcgccgcatctcgggcaagcaggcctatgagtggggcatcgccgtggaatgcgtggcagacgccgagctggaagccgccaccgatgcgctggtcgacgagctgcgcggcttctcgccgctggcgcagcgcaccgccaagaagctgctcaacgacaccgaggacgcaccgctgtcgattgccatcgagctggaagggcattgctatagccgcctgcgcagctcggacgatttccgcgaaggcgtggaagccttccacggcaagcgcaagccggcgttccgcggcagctga

SEQ ID NO: 45 - protein, 449 aa, Cupriavidus basilensis
  misc_feature: Major facilitator superfamily transporter, HMF 14
MEAVAKKSAATISEALPAASNRQVFGAVAASCMGWALDLFDLFILLFVAPVIGRLFFPSEHAMLSLAAVYASFAVTLLMRPLGSAIFGSYADRHGRKGAMVVAVTGVGLSTAAFGLLPTVGQVGLLAPALFILLRLVQGIFVGGVVASTHTIGTESVPPSWRGAVSGLVGGGGAGLGALLASITYMAMTALFPGEAFDAWGWRCMFFSGIISSVLGLFIFNSLEESPLWKQLQAAKGHAAPVENPLRVIFSRQYRGVLFVNILLTVGGGSAYYLTSGYLPTFLKVVVKASAGESAAILMASSLGVIVASILAGHLSTMIGRKRAFLLIGALNVVVLPLLYQWMPAAPDTTTLGLYAVVLSMLGCSGFAPILIFLNERFPTSIRATGTGLSWNIGFAVGGMMPTFASLCASTPAELPMVLGIFLAVVTIIYLVGAFIVPETVGRLGDNGA

SEQ ID NO: 46 - DNA, 1350 bp, Cupriavidus basilensis
  misc_feature: Major facilitator superfamily transporter, HMF 14
atggaagccgtagcaaagaagagtgcagcgacgatcagcgaggcgctgccagcggcgagcaatcgccaggtgtttggtgccgtggcggcgtcgtgcatgggatgggcgctggacctgttcgacctgttcatcctgctgttcgtggcgcccgtgatcggcaggctgtttttcccgtcggagcacgcgatgctgtcgctggcggcggtgtatgcgtcgtttgccgtgacgctgctgatgcggccgctcggctcggcgatcttcggctcttatgccgaccgccacggccgcaagggggcgatggtggttgccgtcactggcgttggcttgtccacggcggcgttcggcctgctgccgacggtgggtcaggtggggctgcttgcgccagccttgtttatcctgctgcggctggtgcagggcatcttcgtgggtggcgtggtggcatccacccacaccatcggtaccgaatcggtgcccccgtcctggcgcggcgccgtttccgggctggttggtggcggtggcgcgggtctcggggcgctgctggcttccattacctacatggcgatgaccgcgctgtttccgggggaagcgttcgatgcctggggttggcgctgcatgttcttctccggcatcatcagctcggtgctcggcctgttcatcttcaactcgctggaggagtctccgctgtggaagcagttgcaggcggccaaggggcacgccgcgccggttgagaacccgctgcgcgtgatcttctcccgccagtaccgtggtgtcctcttcgtcaacatcctgctcaccgtgggcggtggcagcgcctactacctgacctccggctatctgccgaccttcctcaaggtggtggtgaaggcatcggctggcgagtctgccgccatcctgatggccagcagtctgggtgtgatcgtggcatcgattcttgccggccacctcagtacgatgatcggccgcaagcgagccttcctgttgatcggcgcgctgaacgtggtagtactgccgctgctctaccagtggatgccggcggcgccggacaccaccacgctcggcctgtatgctgtggtgctgtccatgctgggctgcagcggcttcgccccgatcctcattttcctgaacgaacggttccccaccagcatccgtgccacggggaccggcctgtcatggaatatcggatttgccgtcggtggcatgatgccgacgtttgcttcgctgtgcgccagcacccctgccgaactgcccatggtgctgggcatcttcctggcggttgtcaccatcatctacctggtgggtgcgttcatcgttccggagacggtagggcgccttggcgacaatggagcgtag